(19) United States
(12) Patent Application Publication
(10) Pub. No.: US 2024/0271121 A1
(43) Pub. Date: Aug. 15, 2024

(54) COMPOSITIONS AND METHODS FOR ISOLATING T CELL RECEPTORS (TCRS) AND CELLS EXPRESSING THE SAME

(71) Applicant: ADOC SSF, LLC, South San Francisco, CA (US)

(72) Inventors: Olivier Dalmas, South San Francisco, CA (US); Jonathan B. Johnston, South San Francisco, CA (US); Elizabeth Speltz, South San Francisco, CA (US)

(73) Assignee: ADOC SSF, LLC, South San Francisco, CA (US)

(21) Appl. No.: 18/633,773

(22) Filed: Apr. 12, 2024

Related U.S. Application Data
(63) Continuation of application No. PCT/US22/46228, filed on Oct. 11, 2022.
(60) Provisional application No. 63/254,658, filed on Oct. 12, 2021.

(51) Int. Cl. C12N 15/10; C07K 14/005; C07K 14/74; C12N 5/0783; C40B 30/04

(52) U.S. Cl. CPC C12N 15/1037; C07K 14/005; C07K 14/70539; C12N 5/0636; C40B 30/04; C07K 2319/02; C12N 2710/20022

(57) ABSTRACT

Disclosed herein are antigenic peptide-MHC molecules, termed comPACT2.0 polypeptides, and methods of producing such molecules. Also disclosed herein are methods of producing libraries of comPACT2.0 polynucleotides and polypeptides, and their exemplary use in capturing cancer neoepitope-reactive T cells.

### CROSS-REFERENCE TO RELATED APPLICATIONS

This application is a continuation application of International Patent Application No. PCT/US22/46228, filed on Oct. 11, 2022, which claims priority to U.S. Provisional Application No. 63/254,658, filed on Oct. 12, 2021, the content of each of which is incorporated in its entirety, and to each of which priority is claimed.

### SEQUENCE LISTING

This application contains a Sequence Listing which has been submitted electronically in XML format and is hereby incorporated by reference in its entirety. Said .XML copy, created on Apr. 12, 2024, is named “087520.0341.xml” and is 343,952 bytes in size. The sequence listing contained in this .XML file is part of the specification and is hereby incorporated by reference herein in its entirety.

### BACKGROUND

T cells are the primary mediators of adaptive immunity. Directed by the specificity of each T cell's unique T cell receptor (TCR), T cells regulate autoimmunity, help activate B cells and innate effectors, and directly kill infected and cancerous cells in a precisely targeted manner.

There is an unmet need for rapid and robust TCR ligand discovery technologies to allow for the isolation and identification of TCRs that can be engineered into human primary cells or used in the making of a vaccine, for example, to treat disease. It has previously been disclosed that peptide-MHC multimers can be used to sort T cells according to the antigenic specificity of their TCRs. This, for example, is an important step in isolating tumor-specific TCRs for cancer cellular therapy.

A typical current peptide-MHC production protocol begins with solid-phase synthesis of the peptide ligand(s) of interest. In parallel, the universal β₂-microglobulin and relevant MHC class I molecules are heterologously expressed in E. coli, yielding misfolded inclusion bodies. Each peptide is added to a refolding reaction containing β2M and the relevant MHC I molecule. Finally, the portion of a ternary complex that refolds correctly can be purified and formulated for use in Peptide-MHC multimer production.

However, the known technology has limitations that do not allow the identification and isolation of all HLA Class I alleles. Specifically, the A33 and B44 MHC Class I alleles show very little expression using the known peptide-MHC technology. This limitation was previously unexplained based on the binding motifs for these alleles and known informatic tools such as AlphaFold and Rosetta. Accordingly, there was no known or anticipated way to redesign the peptide-MHC technology to solve these limitations.

Furthermore, without the ability to capture the A33 and B44 alleles, underrepresented populations will have fewer TCRs identified and in turn are less eligible for therapies. Specifically, the A33:03 allele represents 0% of the Caucasian population, 0% of the Native American population, 2% of the Hispanic population, 19% of the Asian population, and 10% of the African American population; the B44:02 allele represents 17% of the Caucasian population, 14% of the Native American population, 8% of the Hispanic population, 2% of the Asian population, and 4% of the African American population; and the B44:03 allele represents 8% of the Caucasian population, 8% of the Native American population, 12% of the Hispanic population, 10% of the Asian population, and 8% of the African American population. Accordingly, by means of example, the ability to capture TCRs with the A33:03 allele will result in creating therapy options for 19% of the Asian population and 10% of the African American populations that would otherwise not have TCR therapy options.

Disclosed herein are various compositions and processes for producing peptide-MHC multimers that allow for the expression of peptide-MHC complexes that bind to the A33 and B44 MHC Class I alleles and solve the unmet need described herein.

### SUMMARY

The methods and compositions described herein enable rapid identification of antigens targeted by an immune response and isolation of the T cells and thus the TCR sequences mediating that response. The compositions may be applied to identify and isolate antigen-specific T cells and their encoded MHC I-restricted TCRs for drug development. The identification and characterization of antigen-specific T cells may also be applied in the diagnosis, monitoring, and prognosis of immune responses and disease in the context of (pre- and post-) treatment. The methods and compositions described herein have the ability to refine the MHC binding algorithms for less-well studied MHC alleles, improving other targeted immunotherapies such as neoepitope vaccines and cell therapies.

In certain embodiments, the present disclosure provides a polynucleotide comprising, in a 5′ to 3′ orientation, a sequence insert, a beta 2 Microglobulin (β2M) sequence, and a Major Histocompatibility Complex (MHC) allele sequence, wherein the sequence insert comprises a stop codon in each open reading frame, and wherein the MHC allele sequence comprises a Y84C mutation and a A139C mutation. In certain embodiments, the sequence insert is flanked by a first universal target sequence. In certain embodiments, the sequence insert is flanked by a second universal target sequence.

In certain embodiments, the present disclosure provides a polynucleotide comprising, in a 5′ to 3′ orientation, a first universal target sequence, a nucleotide sequence encoding an antigenic peptide, a second universal target sequence, a Beta 2 Microglobulin (β2M) sequence, and a Major Histocompatibility Complex (MHC) allele sequence, wherein the MHC allele sequence comprises a Y84C mutation and a A139C mutation. In certain embodiments, the polynucleotide further comprises a linker positioned 5′ end of the β2M sequence. In certain embodiments, the linker comprises the amino acid sequence set forth in SEQ ID NO: 133 or SEQ ID NO: 134.

In certain embodiments, the present disclosure provides a polynucleotide, in a 5′ to 3′ orientation, a sequence encoding a linker, a Beta 2 Microglobulin (β2M) sequence, and a Major Histocompatibility Complex (MHC) allele sequence, wherein the MHC allele sequence comprises a Y84C mutation and a A139C mutation. In certain embodiments, the linker comprises the amino acid sequence set forth in SEQ ID NO: 133 or SEQ ID NO: 134.

In certain embodiments, the antigenic peptide is a neoantigen. In certain embodiments, the antigenic peptide is a public neoantigen. In certain embodiments, the public neoantigen is a HPV neoantigen. In certain embodiments, the HPV neoantigen is derived from E16 protein or E17 protein. In certain embodiments, the antigenic peptide is a private neoantigen.

In certain embodiments, the β2M allele is a human β2M allele. In certain embodiments, the MHC allele is a human MHC allele. In certain embodiments, the human MHC allele is an HLA allele selected from the group consisting of: HLA-A*01:01, HLA-A*02:01, HLA-A*03:01, HLA-A*24:02, HLA-A*30:02, HLA-A*31:01, HLA-A*32:01, HLA-A*33:01, HLA-A*68:01, HLA-A*11:01, HLA-A*23:01, HLA-A*30:01, HLA-A*33:03, HLA-A*25:01, HLA-A*26:01, HLA-A*29:02, HLA-A*68:02, HLA-B*07:02, HLA-B*14:02, HLA-B*18:01, HLA-B*27:02, HLA-B*39:01, HLA-B*40:01, HLA-B*44:02, HLA-B*46:01, HLA-B*50:01, HLA-B*57:01, HLA-B*58:01, HLA-B*08:01, HLA-B*15:01, HLA-B*15:03, HLA-B*35:01, HLA-B*40:02, HLA-B*42:01, HLA-B*44:03, HLA-B*51:01, HLA-B*53:01, HLA-B*13:02, HLA-B*15:07, HLA-B*27:05, HLA-B*35:03, HLA-B*37:01, HLA-B*38:01, HLA-B*41:02, HLA-B*44:05, HLA-B*49:01, HLA-B*52:01, HLA-B*55:01, HLA-C*02:02, HLA-C*03:04, HLA-C*05:01, HLA-C*07:01, HLA-C*01:02, HLA-C*04:01, HLA-C*06:02, HLA-C*07:02, HLA-C*16:01, HLA-C*03:03, HLA-C*07:04, HLA-C*08:01, HLA-C*08:02, HLA-C*12:02, HLA-C*12:03, HLA-C*14:02, HLA-C*15:02, and HLA-C*17:01. In certain embodiments, the HLA allele comprises the amino acid sequence selected from the group consisting of SEQ ID NOs: 1-66. In certain embodiments, the HLA allele consists of the amino acid sequence selected from the group consisting of SEQ ID NOs: 1-66. In certain embodiments, the HLA allele comprises the amino acid sequence selected from the group consisting of SEQ ID NOs: 137-202. In certain embodiments, the HLA allele consists of the amino acid sequence selected from the group consisting of SEQ ID NOs: 137-202.

In certain embodiments, the human MHC allele is selected from the group consisting of HLA-A*33:01, HLA-A*33:03, HLA-B*44:02, HLA-B*44:03, and HLA-B*44:025. In certain embodiments, the human MHC allele is HLA-A*33:01. In certain embodiments, the human MHC allele is HLA-A*33:03. In certain embodiments, the human MHC allele is HLA-B*44:02. In certain embodiments, the human MHC allele is HLA-B*44:03. In certain embodiments, the human MHC allele is HLA-B*44:025.

In certain embodiments, the first and second universal target sequences comprise a polymerase chain reaction (PCR) primer target site. In certain embodiments, the first and second universal target sequences comprise a restriction enzyme cleavage site. In certain embodiments, the polynucleotide further comprises a sequence encoding a signal sequence. In certain embodiments, the polynucleotide further comprises a purification cluster sequence positioned at the 3′ end of the polynucleotide, wherein the purification cluster comprises a first affinity tag sequence, a protease cleavage site sequence, and a second affinity tag sequence.

In certain embodiments, the present disclosure provides an expression construct, a vector, or a host cell comprising the polynucleotide disclosed herein. In certain embodiments, the present disclosure provides a polypeptide encoded by the polynucleotide disclosed herein.

In certain embodiments, the present disclosure provides an protein complex comprising a first polypeptide comprising an antigenic peptide disclosed herein and a second polypeptide encoded by the polynucleotide disclosed herein.

In certain embodiments, the present disclosure provides an library comprising at least two polynucleotide, at least two expression constructs, at least two vectors, at least two polypeptides, at least two protein complexes, or at least two host cells disclosed herein, wherein at least one polynucleotide, at least one expression construct, at least one vector, at least one polypeptide, or at least one cell comprises a human MHC allele selected from the group consisting of HLA-A*33:01, HLA-A*33:03, HLA-B*44:02, HLA-B*44:03, and HLA-B*44:025. In certain embodiments, the library comprises comprising at least 66 different polynucleotide molecules, 66 different expression constructs, 66 different vectors, 66 different polypeptides.

In certain embodiments, the present disclosure provides a kit comprising a polynucleotide, an expression construct, a vector, a polypeptide, a protein complex, a host cell, or a library of disclosed herein and instructions for use.

In certain embodiments, the present disclosure provides a method for isolating an antigen-specific T cell, the method comprising: a) providing a plurality of particles, wherein each particle comprises a polypeptide or a protein complex disclosed herein; b) obtaining a sample known or suspected to comprise one or more T cells; c) contacting the plurality of particles with the sample, wherein the contacting comprises providing conditions sufficient for a single T cell to bind the polypeptide attached to the particle; and d) isolating the single T cell associated with the particle.

### BRIEF SUMMARY OF DRAWINGS

These and other features, aspects, and advantages of disclosed compositions and methods will become better understood with regard to the following description, and accompanying drawings, where:

FIG. 1A & FIG. 1B. FIGS. 1A and 1B show comparisons between comPACT1.0 and comPACT2.0 structures. FIG. 1A shows a diagram of the previously disclosed comPACT1.0 that has functional limitations for at least the A33 and B44 alleles. FIG. 1B shows a diagram of the new comPACT2.0 disclosed herein that solves the problems and limitations of use of comPACT1.0.

FIG. 2A & FIG. 2B. FIGS. 2A and 2B show that comPACT2.0 is capable of binding alleles such as A02:01 which previously bound well to comPACT1.0 and overcomes the limitations of comPACT1.0 by binding A33:03. FIG. 2A shows that there is some improvement to the A02:01 binding with comPACT2.0 but also an unexpected significant increase in binding of the A33:03 allele (6% with comPACT1.0 compared to 99% with comPACT2.0). FIG. 2B shows that the expression rate of good and bad comPACT1.0 binders was significantly improved with comPACT2.0. As shown, the production of the A02:01 comPACTs approximately doubled between the comPACT1.0 and comPACT2.0 structure and the production of the A33:03 comPACTs was not possible using the comPACT1.0 structure but was possible, with a significant yield, using the comPACT2.0 structure.

FIG. 3. FIG. 3 shows the success rate of comPACT1.0 across multiple representative alleles. As shown, comPACT1.0 was not successful for A33:01 and A33:03 and had poor results for others. Based on this data and similar data for other alleles not shown, there was a clear need to invent a comPACT2.0 to allow for the inclusion of more alleles into the library.

FIG. 4. FIG. 4 shows the improved yield generated by comPACT2.0 for multiple alleles associated with multiple neoepitopes.

FIG. 5. FIG. 5 shows the different linkers tested in the design of comPACT2.0 and their effect on hit rate.

FIG. 6. FIG. 6 shows the structure of the comPACT2.0 and the empty comPACT2.0.

FIGS. 7A & FIG. 7B. FIG. 7A shows a representative graph illustrating the IFNγ secretion after stimulation with different concentrations of cognate neoE-HLA generated as either peptide-pulsed “empty” comPACT2.0 or neoE-HLA expressed as a Single Chain Trimer (SCT) comPACT1.0. FIG. 7B shows a representative plot illustrating the IFNγ secretion after stimulation with different concentrations of cognate neoE-HLA expressed as a Single Chain Trimer (SCT) in the comPACT 2.0 or comPACT 1.0 constructs.

FIGS. 8A-8C. FIGS. 8A-8C show a representative graph illustrating equivalent multimer binding with cognate neoE-HLA-A02:01 generated as either peptide-pulsed “empty” HLA comPACT 2.0 (top row) or neoE-HLA expressed as a Single Chain Trimer (SCT) HLA comPACT 1.0 (bottom row). FIG. 8A shows graphs of NeoTCR targeting the HPV16-E7 antigen. FIG. 8B shows graphs of NeoTCR targeting the Neo12 antigen. FIG. 8C shows graphs of NeoTCR targeting the HPV16-E6 antigen.

### DETAILED DESCRIPTION

The present disclosure provides compositions and methods for detection, identification, and isolation of T cell receptors and cells comprising the same (e.g., CD8 T cells). The present disclosure is based, in part, on the discovery that certain amino acid sequences could stabilize the expression of the A33 and B44 MHC Class I alleles, which traditionally show very little expression using peptide-MHC technologies.

Non-limiting embodiments of the present disclosure are described by the present description and examples. For purposes of clarity of disclosure and not by way of limitation, the detailed description is divided into the following subsections:
1. Definitions;
2. Nucleotide and Peptide Compositions;
3. Expression Constructs and Vectors;
4. Host Cells;
5. Libraries;
6. Particles;
7. Methods of Producing;
8. T Cell Isolation; and
9. Exemplary Embodiments.

### 1. Definitions

Unless defined otherwise, all technical and scientific terms used herein have the meaning commonly understood by a person skilled in the art. The following references provide one of skill with a general definition of many of the terms used in the presently disclosed subject matter: Concise Medical Dictionary, edited by Law and Martin, Oxford University Press, 2020; A Dictionary of Biology, edited by Hine, Oxford University Press, 2019; A Dictionary of Chemistry, edited by Law and Rennie, Oxford University Press, 2020; Oxford Dictionary of Biochemistry and Molecular Biology, edited by Cammack, Atwood, Campbell, Parish, Smith, Vella, and Stirling, Oxford University Press, 2006; and Paul, William. 2013. Fundamental Immunology. Philadelphia, PA: Wolters Kluwer Health/Lippincott Williams & Wilkins. As used herein, the following terms have the meanings ascribed to them below, unless specified otherwise.

It is understood that aspects and embodiments of the invention described herein include “comprising,” “consisting,” and “consisting essentially of” aspects and embodiments.

The terms “comprises” and “comprising” are intended to have the broad meaning ascribed to them in U.S. Patent Law and can mean “includes,” “including” and the like.

As used herein, “sequence identity” or “identity” (in the context of two nucleic acid or polypeptide sequences) refers to the residues in the two sequences that are the same when aligned for maximum correspondence over a specified comparison window. When the percentage of sequence identity is used about proteins it is recognized that residue positions that are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art.

As used herein, “percentage of sequence identity” refers to the value determined by comparing two optimally aligned sequences over a comparison window, wherein the portion of the polynucleotide sequence in the comparison window can comprise additions or deletions (i.e., gaps) as compared to the reference sequence (which does not comprise additions or deletions) for the optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Methods of alignment of sequences for comparison include, without any limitation, the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 87:2264, modified as in Karlin and Altschul (1993) Proc. Natl. Acad. Sci. USA 90:5873-5877. Computer implementations of these mathematical algorithms include, without any limitation, CLUSTAL, CLUSTALW, CLUSTALOMEGA, ALIGN, ALIGN PLUS, GAP, BESTFIT, BLAST, FASTA, TFASTA, BLASTN, BLASTX, BLASTP, TBLASTN, and TBLASTX.

A “conservative substitution” or a “conservative amino acid,” refers to the substitution of amino acid with a chemically or functionally similar amino acid. Conservative substitution tables providing similar amino acids are well known in the art. In certain embodiments, acidic amino acids D and E are conservative substitutions for one another; basic amino acids K, R, and H are conservative substitutions for one another; hydrophilic uncharged amino acids S, T, N. and Q are conservative substitutions for one another; aliphatic uncharged amino acids G, A, V, L, and I are conservative substitutions for one another; non-polar uncharged amino acids C, M, and P are conservative substitutions for one another; aromatic amino acids F, Y, and W are conservative substitutions for one another; A, S, and T are conservative substitutions for one another; D and E are conservative substitutions for one another; N and Q are conservative substitutions for one another; R and K are conservative substitutions for one another; I, L, and M are conservative substitutions for one another; F, Y, and W are conservative substitutions for one another; A and G are conservative substitutions for one another; D and E are conservative substitutions for one another; N and Q are conservative substitutions for one another; R, K and H are conservative substitutions for one another; I, L, M, and V are conservative substitutions for one another; F, Y and W are conservative substitutions for one another; S and T are conservative substitutions for one another; and C and M are conservative substitutions for one another. Additional conservative substitutions may be found, for example, in Creighton, Proteins: Structures and Molecular Properties 2nd ed. (1993) W. H. Freeman & Co., New York, NY.

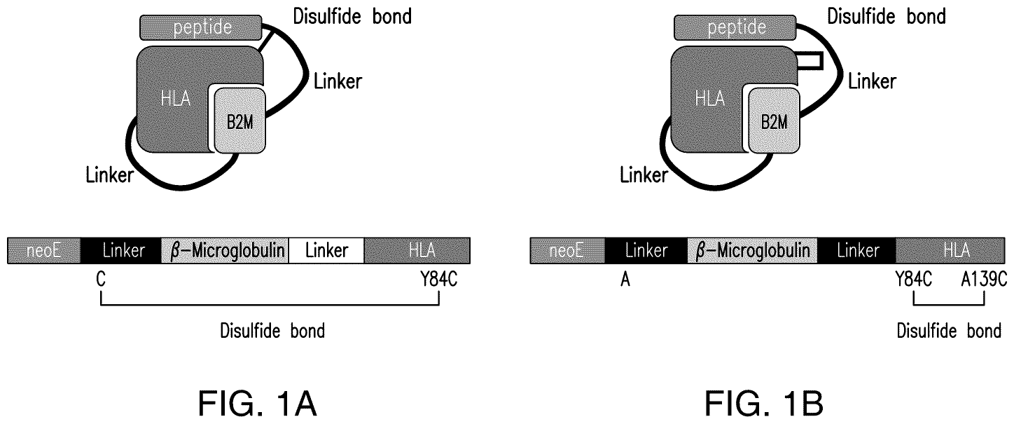

As used herein, the terms “antigenic sequence” and the reference to the neoepitope (neoE) and peptide in FIGS. 1A and 1B are used interchangeably and refer to the peptide included in the comPACT2.0 single chain trimer polypeptide (and the polynucleotide encoding the polypeptide). As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

The terms “Cancer” and “Tumor” are used interchangeably herein. As used herein, the terms “Cancer” or “Tumor” refer to all neoplastic cell growth and proliferation, whether malignant or benign, and all pre-cancerous and cancerous cells and tissues. The terms are further used to refer to or describe the physiological condition in mammals that is typically characterized by unregulated cell growth/proliferation. Cancer can affect a variety of cell types, tissues, or organs, including but not limited to an organ selected from bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Cancer includes cancers, such as sarcomas, carcinomas, or plasmacytomas (malignant tumor of the plasma cells). Examples of cancer include, but are not limited to, those described herein. The terms “Cancer” or “Tumor” and “Proliferative Disorder” are not mutually exclusive as used herein.

“comPACT” and “comPACT polypeptide” as used herein mean a peptide comprising an antigen or epitope amino acid sequence (“epitope peptide”), a β2-microglobulin protein sequence, and an extracellular domain sequence of an MHC heavy chain. comPACT refers generally to both comPACT1.0 and comPACT2.0. The β2-microglobulin protein sequence and extracellular domain sequence of the MHC heavy chain are together herein referred to as an “HLA complex” when they are correctly folded, including, e.g., proper formation of intramolecular disulfide bonds. In some embodiments, the antigen or epitope is a neoantigen or neoepitope. In some embodiments, the β2-microglobulin protein sequence is a full-length β2-microglobulin protein sequence. In some embodiments, the extracellular domain of an MHC heavy chain is the extracellular domain of a class I MHC heavy chain comprising α1, α2, and α3 domains. In some embodiments, the comPACT polypeptide comprises a binding moiety conjugation site and/or a binding moiety conjugated to the site. In some such embodiments, the comPACT polypeptide comprises a biotin conjugation site, e.g., an AviTag and/or a biotin-conjugated to the site. In some embodiments, the comPACT polypeptide comprises a cleavable tag and a purification tag, such as a TEV tag and histidine tag, respectively. In some embodiments, the comPACT polypeptide comprises a signal sequence, such as an N-terminal signal sequence. In some embodiments, the comPACT polypeptide comprises one or more linkers between the antigen or epitope sequence, the β2-microglobulin protein sequence, the extracellular domain sequence of the MHC heavy chain, and/or a biotin conjugation site sequence.

“comPACT1.0” refers to a previously disclosed peptide as described in International Patent Application No. PCT/US2019/025415. comPACT1.0 peptides cannot be made for at least HLA A33 and B44 alleles and have reduced capacity for many other HLA alleles.

“comPACT2.0” refers to the set of peptides disclosed herein that were invented and designed to mitigate the limitations of comPACT technology caused by comPACT1.0. comPACT2.0 can be made for alleles such as A33 and B44 which was not possible with the comPACT1.0 technology. “comPACT Library” means one or more comPACT.

“Neoantigen” refers to an antigen that has at least one alteration that makes the neoantigen or presentation of the neoantigen distinct from its corresponding wild-type antigen, e.g., mutations in the polypeptide sequence, differences is post-translation modifications, or differences in expression level. “Neoantigen” and “Tumor Neoantigen” mean a specific antigen on a cell that is used as an identifying target for killing. As applied to cancer and tumors, a neoantigen is an antigen that is specific to the tumor or cancer. As applied to pathogens and pathogen-infected cells, a neoantigen is an antigen that is specific to the pathogen or pathogen-infected cell. “Tumor neoantigens” refers to neoantigens that are derived from a tumor or cancer, e.g., from the tumor of a patient.

In certain embodiments, the term “neoantigen”, “neoepitope” or “neoE” refer to a newly formed antigenic determinant that arises, e.g., from a somatic mutation(s) and is recognized as “non-self” In certain embodiments, a mutation giving rise to a “neoantigen”, “neoepitope” or “neoE” can include a frameshift or non-frameshift indel, missense or nonsense substitution, splice site alteration (e.g., alternatively spliced transcripts), genomic rearrangement or gene fusion, any genomic or expression alterations, or any post-translational modifications. In certain embodiments, the neoantigen can be a private neoantigen. As used herein, the term “private neoantigen” refers to neoantigens that are exclusively expressed and present in a subject having certain cancer. For clarity, a private neoantigen is a neoantigen that cannot be used for another subject. In certain embodiments, the neoantigen can be a “public neoantigen.” The term “public neoantigen,” as used herein, refers to neoantigens that are shared by more than one subject. “neoTCR Product,” “neoTCR T Cell therapy,” “neoTCR T Cell treatment,” and “neoTCR T Cell” are used interchangeably and all refer to the genetically engineered T cell expressing a TCR that recognizes the neoepitope that was identified and designed using comPACT polypeptides and polynucleotides and the imPACT Isolation Technology described in U.S. Patent Publication No. 2020/0256849, the content of which is incorporated by reference in its entirety.

“Neo12” and “Neo12 protein” means an exemplary neoepitope.

### 2. Nucleotide and Peptide Compositions

T-cell mediated immunity is characterized by the activation of antigen-specific cytotoxic T cells that are able to induce death in cells that display antigen in a major histocompatibility complex (MHC) on their surface. These cells displaying an MHC complex loaded with antigen include virus-infected cells, cells with intracellular bacteria, cells that have internalized or phagocytosed extracellular sources of protein, and cancer cells displaying tumor antigens.

A natural class I MHC heavy chain comprises about 350 amino acids; a natural β2-microglobulin comprises about 100 amino acids, and a class I antigen peptide typically has a length of from about 7 to about 15 amino acids. Class I heavy chains are encoded by genes of the major histocompatibility complex, designated HLA-A, -B, and -C in humans, and H-2K, D, and L in mice. The class I heavy chains and β2-microglobulin are separately encoded on different chromosomes. Antigen peptides are normally processed by cells from protein sources such as, for example, viruses, bacteria, or cancer cells. Diverse variants have been identified for the polypeptides encoded by the HLA-A, -B, and -C MHC genes in humans, as well as the murine H-2K, D, and L MHC genes.

Certain embodiments of the method disclosed herein are directed to a method of manufacturing a single molecule in which a selected antigen (e.g., a neoantigen) is linked to an MHC complex comprising a β2-microglobulin (β2M) and an MHC heavy chain. Different MHC heavy chains are linked to the β2M molecule to form a varying number of MHC templates. The methods disclosed herein of inserting an antigen into an MHC template via restriction digest or PCR-based assembly by utilizing universal target sequences flanking the antigen insertion site results in the ability to construct a library of different antigen-MHC complexes in a high-throughput method that are, e.g., personalized for a given patient.

In certain embodiments, an MHC display moiety can include a recombinant MHC molecule. In certain embodiments, the antigen-MHC complex formed by a comPACT2.0 results in the display of the antigens such that they are capable of recognition by a cognate TCR molecule. In certain embodiments, the MHC complex is an MHC Class I (MHC I) complex that pairs with CD8-positive (CD8+) T “killer” cells. In certain embodiments, the MHC complex is an MHC Class 11 (MHC II) complex that pairs with CD4-positive (CD4+) T cells. The MHC allele encoded in each comPACT2.0 is easily swapped out for other MHC alleles, enabling antigenic interrogation of T cells from patients of any MHC haplotype.

In certain embodiments, the MHC class I heavy chain sequence of a comPACT2.0 can include one or more amino acid substitutions, additions, and/or deletions, such as a substitution of Tyr-84 with a non-aromatic amino acid other than proline. In these embodiments, the amino acid substitution is an amino acid encoded by the standard genetic code such as leucine, isoleucine, valine, serine, threonine, alanine, histidine, glutamine, asparagine, lysine, aspartic acid, glutamic acid, cysteine, arginine, serine or glycine, or is a modified or unusual amino acid. In certain embodiments, the MHC class I heavy chain sequence of a comPACT2.0 comprises a tyrosine-84 to alanine substitution. In certain embodiments, the MHC class I heavy chain sequence of a comPACT2.0 comprises a Tyrosine-84 to cysteine substitution.

In certain embodiments, the MHC allele lacks a transmembrane domain. In certain embodiments, the MHC allele lacks a cytoplasmic domain. In certain embodiments, the MHC allele lacks a transmembrane and a cytoplasmic domain. In certain embodiments, the HLA allele lacks a transmembrane domain. In certain embodiments, the HLA allele lacks a cytoplasmic domain. In certain embodiments, the HLA allele lacks a transmembrane and a cytoplasmic domain.

Any MHC or HLA allele may be used in the comPACT2.0 described herein. Exemplary HLA alleles include, but are not limited to, HLA-A*01:01, HLA-A*02:01, HLA-A*03:01, HLA-A*24:02, HLA-A*30:02, HLA-A*31:01, HLA-A*32:01, HLA-A*33:01, HLA-A*68:01, HLA-A*11:01, HLA-A*23:01, HLA-A*30:01, HLA-A*33:03, HLA-A*25:01, HLA-A*26:01, HLA-A*29:02, HLA-A*68:02, HLA-B*07:02, HLA-B*14:02, HLA-B*18:01, HLA-B*27:02, HLA-B*39:01, HLA-B*40:01, HLA-B*44:02, HLA-B*46:01, HLA-B*50:01, HLA-B*57:01, HLA-B*58:01, HLA-B*08:01, HLA-B*15:01, HLA-B*15:03, HLA-B*35:01, HLA-B*40:02, HLA-B*42:01, HLA-B*44:03, HLA-B*51:01, HLA-B*53:01, HLA-B*13:02, HLA-B*15:07, HLA-B*27:05, HLA-B*35:03, HLA-B*37:01, HLA-B*38:01, HLA-B*41:02, HLA-B*44:05, HLA-B*49:01, HLA-B*52:01, HLA-B*55:01, HLA-C*02:02, HLA-C*03:04, HLA-C*05:01, HLA-C*07:01, HLA-C*01:02, HLA-C*04:01, HLA-C*06:02, HLA-C*07:02, HLA-C*16:01, HLA-C*03:03, HLA-C*07:04, HLA-C*08:01, HLA-C*08:02, HLA-C*12:02, HLA-C*12:03, HLA-C*14:02, HLA-C*15:02, and HLA-C*17:01. Any other suitable HLA allele known in the art may be used in the comPACT2.0 described herein.

In certain embodiments, HLA alleles that could not be used in the comPACT1.0 technology such as the A33 and B44 alleles can be used in the comPACT2.0 technology. In certain embodiments, comPACT2.0 libraries are made with A33 and B44 alleles that were previously not possible for the comPACT1.0 libraries.

In certain embodiments, the β2-microglobulin (β2M) may include a recombinant β2M molecule. In certain embodiments, the β2M sequence may include one or more amino acid substitutions, additions, and/or deletions as described above. In certain embodiments, this substitution comprises a Serine-88 to Cysteine substitution.

In certain embodiments, the comPACT2.0 polypeptides comprise two cysteine substitutions in the HLA portion of the polypeptide (FIG. 1B). In certain embodiments, the two cysteine substitutions are Y84C and A139C. In certain embodiments, the Y84C and A139C substitutions result in a disulfide bond. In certain embodiments, the disulfide bond formed by the Y84C and A139C substitutions stabilizes the comPACT2.0 and allows the comPACT2.0 to be used with the A33 and A44 alleles.

In certain exemplary embodiments, the comPACT2.0 polynucleotide comprises, in a 5′ to 3′ orientation, (i) a promoter sequence, (ii) a first universal target sequence, (iii) the nucleotide sequence encoding an antigenic peptide, wherein the antigenic peptide is a tumor neoantigen, (iv) a second universal target sequence, (v) a β2M sequence, (vi) an MHC allele sequence, (vii) a first affinity tag sequence, (viii) a protease cleavage site sequence, and (ix) a second affinity tag sequence. In certain embodiments, the MHC allele sequence comprises a sequence selected from the group consisting of the sequences shown in SEQ ID NOs: 1-66 or SEQ ID NO: 137-202. In certain embodiments, the MHC allele sequence comprises a sequence encoding an amino acid sequence selected from the group consisting of the sequences shown in SEQ ID NOs: 1-66 or SEQ ID NO: 137-202, (vii) a first affinity tag sequence, (viii) a protease cleavage site sequence, and (ix) a second affinity tag. In certain embodiments, the MHC allele sequence comprises a sequence selected from the group consisting of the sequences shown in SEQ ID NOs: 1-66 or SEQ ID NO: 137-202, (vii) a first affinity tag sequence, (viii) a protease cleavage site sequence, and (ix) a second affinity tag.

In certain embodiments, the comPACT2.0 polypeptide comprises, in a N-end to C-end orientation, (i) a promoter peptide, (ii) a first universal target peptide, (iii) an antigenic peptide, wherein the antigenic peptide is a tumor neoantigen, (iv) a second universal target peptide, (v) a β2M peptide, and (vi) an MHC peptide comprising a sequence shown in SEQ ID NOs: 1-66 or SEQ ID NO: 137-202. In certain embodiments, the comPACT2.0 polypeptide comprises, in a 5′ to 3′ orientation, (i) a promoter peptide, (ii) a first universal target peptide, (iii) a nucleotide sequence encoding an antigenic peptide, wherein the antigenic peptide is a tumor neoantigen, (iv) a second universal target peptide, (v) a β2M peptide, and (vi) an MHC peptide comprising a sequence shown in SEQ ID NOs: 1-66 or SEQ ID NO: 137-202.

In certain embodiments, the present disclosure provides polynucleotides encoding a comPACT2.0 polypeptide, the polynucleotide comprising, in a 5′ to 3′ orientation, a sequence insert, a beta 2 Microglobulin (β2M) sequence, and a Major Histocompatibility Complex (MHC) allele sequence. In certain embodiments, the sequence insert comprises a stop codon in each open reading frame. In certain embodiments, the MHC allele sequence comprises a Y84C mutation and an A139C mutation.

In certain embodiments, the present disclosure provides polynucleotides encoding a comPACT2.0 polypeptide, the polynucleotide comprising, in a 5′ to 3′ orientation, a first universal target sequence, a nucleotide sequence encoding an antigenic peptide, a second universal target sequence, a Beta 2 Microglobulin (β2M) sequence, and a Major Histocompatibility Complex (MHC) allele sequence. In certain embodiments, the MHC allele sequence comprises a Y84C mutation and an A139C mutation.

In certain embodiments, the present disclosure provides polynucleotides encoding a comPACT2.0 polypeptide, the polynucleotide comprising, in a 5′ to 3′ orientation, a linker, a Beta 2 Microglobulin (β2M) sequence, and a Multiple Histocompatibility Complex Major Histocompatibility Complex (MHC) allele sequence. In certain embodiments, the MHC allele sequence comprises a Y84C mutation and an A139C mutation.

Additionally, the present disclosure provides polypeptides encoded by the presently disclosed polynucleotides.

In certain embodiments, the present disclosure provides “peptide bound comPACT2.0” polypeptides. As used herein, the term “peptide bound comPACT2.0” refers to polypeptides including an antigenic sequence (e.g., a neoantigen) covalently bound to a Beta 2 Microglobulin (β2M) sequence, and a Major Histocompatibility Complex (MHC) allele sequence (e.g., an MHC allele sequence comprising a Y84C mutation and an A139C mutation).

In certain embodiments, the present disclosure provides “empty comPACT2.0” polypeptides. As used herein, the term “empty comPACT2.0” refers to polypeptides including a linker sequence (e.g., comprising the amino acid sequence set forth in SEQ ID NO: 133 or SEQ ID NO: 134) bound to a Beta 2 Microglobulin (β2M) sequence, and a Major Histocompatibility Complex (MHC) allele sequence (e.g., an MHC allele sequence comprising a Y84C mutation and an A139C mutation).

In certain embodiments, the present disclosure provides “peptide loaded comPACT2.0” polypeptides. As used herein, the term “peptide loaded comPACT2.0” refers to a protein complex comprising a first polypeptide including an antigenic sequence (e.g., a neoantigen) and a second polypeptide including a linker sequence (e.g., comprising the amino acid sequence set forth in SEQ ID NO: 133 or SEQ ID NO: 134) bound to a Beta 2 Microglobulin (β2M) sequence, and a Major Histocompatibility Complex (MHC) allele sequence (e.g., an MHC allele sequence comprising a Y84C mutation and an A139C mutation). For clarity, in a peptide loaded comPACT2.0, the antigenic peptide is not covalently bound to the Beta 2 Microglobulin (β2M) sequence.

### 1.1. Sequence Insert

In certain embodiments, the presently disclosed polynucleotides comprise a sequence insert. In certain embodiments, the sequence insert comprises a stop codon in each open reading frame. As used herein, a “stop codon” or “termination codon” is a nucleotide triplet within a messenger RNA or polynucleotide that signals the termination of the translation process of a protein. Most codons in messenger RNA correspond to the addition of an amino acid to a growing polypeptide chain, which can ultimately become a protein; stop codons signal the termination of this process by binding release factors, which cause the ribosomal subunits to disassociate, releasing the amino acid chain. In certain embodiments, the stop codon in each open reading frame reduces the unwanted synthesis of polypeptides lacking an antigenic sequence (e.g., a private or a public neoantigen).

In certain embodiments, the sequence insert further comprises one or more restriction sites. In certain embodiments, the use of nucleases or restriction enzymes allows the insertion of a polynucleotide encoding an antigenic sequence (e.g., a private or a public neoantigen). In certain embodiments, insertion of a polynucleotide encoding an antigen sequence results in disruption of the stop codon in each open reading frame. In certain embodiments, insertion of a polynucleotide encoding an antigenic sequence allows the synthesis of a comPACT2.0 polypeptide.

In certain embodiments, the use of nucleases or restriction enzymes allows replacing the sequence insert with a polynucleotide encoding an antigenic sequence (e.g., a private or a public neoantigen). In certain embodiments, replacing the sequence insert with a polynucleotide encoding an antigenic sequence allows the synthesis of a comPACT2.0 polypeptide.

### 1.2. Antigenic Sequences

In certain embodiments, the “antigenic sequence” (also referred to as the “peptide” and “neoE” in FIGS. 1A and 1B) includes but is not limited to a tumor antigen, a neoantigen, a tumor neoantigen, a viral antigen, bacterial antigen, phosphoantigen, or a microbial antigen. In certain embodiments, the antigenic peptide is a neoantigen. In certain embodiments, the antigenic peptides are selected from patient data to select antigens with one or more somatic mutations.

In certain embodiments, the neoantigen is a private neoantigen. In certain embodiments, the neoantigen is a public neoantigen. In certain embodiments, the public neoantigen is an HPV neoantigen. In certain embodiments, the HPV neoantigen is derived from an HPV16-E7 polypeptide. In certain embodiments, the HPV16-E7 polypeptide comprises the amino acid sequence set forth in SEQ ID NO: 135. In certain embodiments, the HPV neoantigen is derived from an HPV16-E6 polypeptide. In certain embodiments, the HPV16-E6 polypeptide comprises the amino acid sequence set forth in SEQ ID NO: 136.

### 1.3. Universal Target Sequences

In certain embodiments, the presently disclosed polynucleotides comprise a universal target sequence. In certain embodiments, the antigenic peptide is flanked by universal target sequences or portions thereof. In certain embodiments, the universal target sequences allow for rapid, high throughput methods for replacing or inserting the antigenic peptide encoding nucleotide in the polynucleotide MHC template. In certain embodiments, the universal target sequences may comprise restriction sites for restriction digest-based cloning. In certain embodiments, the restriction sites include, but are not limited to, NotI, BamHI, BlpI, BspEI, BstBI, Xbal, HindIII, EcoRI, ApaI, NotI, and any combination thereof. In certain embodiments, one or more universal target sequences are not present in the genetic material being manipulated, e.g., to reduce or eliminate off-target effects and/or to increase specificity.

In certain embodiments, the universal target sequences comprise polymerase chain reaction (PCR) primer target sequences or primer binding sites. In certain embodiments, universal primer sequences known in the art may be used in the compositions and methods disclosed herein, or the sequences may be different than the previously described universal primer sequences.

In certain embodiments, the polynucleotide encoding comPACT2.0 comprises a first universal target sequence and a second universal target sequence. In certain embodiments, the first universal target sequence and/or the second universal target sequences comprise restriction enzyme cleavage sites.

### 1.2. Linkers

In certain embodiments, the presently disclosed polynucleotides comprise a sequence encoding a linker. In certain embodiments, the linker comprises the amino acid sequence set forth in SEQ ID NO: 133 or SEQ ID NO: 134. In certain embodiment, the linker consists of the amino acid sequence set forth in SEQ ID NO: 133 or SEQ ID NO: 134. In certain embodiments, the linker stabilizes the comPACT2.0 polypeptide. For example, as shown in FIGS. 1A and 1B, the presently disclosed linker (e.g., comprising the amino acid sequence set forth in SEQ ID NO: 133 or SEQ ID NO: 134) stabilizes the comPACT2.0 polypeptide by avoiding disulfide bonds with the MHC allele.

In certain embodiments, a comPACT2.0 polypeptide can comprise a first flexible linker interposed between the antigenic peptide segment and the β2-microglobulin segment. In certain embodiments, the linkers can extend from and connect the carboxyl terminal of the antigenic peptide segment to the amino terminal of the β2-microglobulin segment, or vice versa. In certain embodiments, when a comPACT2.0 polypeptide is expressed, the linked peptide ligand can fold into the binding groove resulting in a functional comPACT2.0 polypeptide.

In certain embodiments, a comPACT2.0 polypeptide can comprise a second flexible linker interposed between the β2-microglobulin segment and the MHC heavy chain segment. In certain embodiments, the second flexible linker can extend from and connect the carboxyl terminal of the β2-microglobulin segment to the amino terminal of the heavy chain segment, or vice versa. In certain embodiments, when a comPACT2.0 polypeptide is expressed, the β2-microglobulin and the heavy chain can fold into the binding groove resulting in a molecule that can function in promoting T cell expansion.

In certain embodiments, the linker(s) used in comPACT1.0 polypeptides are different than the linker(s) used in comPACT2.0 polypeptides. In certain embodiments, the linker used for comPACT2.0 polypeptides is GAGAS(G4S)2 (SEQ ID NO: 134). In certain embodiments, the linker used for comPACT2.0 polypeptides is GGGAS(G4S)2 (SEQ ID NO: 133). In certain embodiments, the GGGAS(G4S)2 linker is selected for the comPACT2.0 polypeptides if the polypeptide comprises the HLA A33:01, HLA A33:03, HLA B44:02, or HLA B44:03 allele. In certain embodiments, a comPACT2.0 library comprising comPACT2.0 polypeptides made using one or more of the HLA A33:01, HLA A33:03, HLA B44:02, or HLA B44:03 alleles comprises comPACT2.0 polypeptides with the GGGAS(G4S)2 linker (SEQ ID NO: 133).

### 1.3. Signal Sequences

In certain embodiments, the presently disclosed polynucleotides comprise a sequence encoding a signal sequence. “Signal sequence,” as used herein, is a peptide that can be included at the N-terminus of a newly synthesized protein to traffic the newly synthesized protein to its intended and/or engineered location inside or outside of the cell. In certain non-limiting embodiments, the signal sequence is a human growth hormone (HGH) signal sequence.

In certain embodiments, the comPACT2.0 polypeptide may comprise a signal sequence. In certain embodiments, the signal sequence is a secretion signal sequence.

### 1.4. Promoters

In certain embodiments, the presently disclosed polynucleotides comprise a sequence encoding a promoter. “Promoter region” or “promoter,” as used herein, means the region of a construct that encodes a promoter that controls the expression of the gene of interest.

In certain embodiments, the promoter is a eukaryotic promoter, a mammalian promoter, a viral promoter, a synthetic promoter, a minimal promoter, a hybrid promoter, a tissue specific promoter, an inducible promoter, or a constitutive promoter.

In certain embodiments, the promoter is a constitutive promoter. For example, but without any limitation, the constitutive promoter is the EF-1α promoter, the hACTB promoter, the hPGK promoter, the MND promoter, or the U6 promoter.

In certain embodiments, a polynucleotide encoding a comPACT2.0 polynucleotide may further comprise a promotor, e.g., for transcription of an mRNA transcript that is translated by a host cell. Promoters may be prokaryotic or eukaryotic (e.g., mammalian) in origin. In certain embodiments, any appropriate promoter for gene transcription in a cell may be used.

In certain embodiments, the 5′ end of the polynucleotide sequence further comprises a promoter sequence linked to the 5′ end of the first universal target.

### 1.5. Affinity Tags

In certain embodiments, the presently disclosed polynucleotides comprise a sequence encoding an affinity tag. As used herein, “affinity tag” refers to unique proteins and/or peptides that are attached at the N- or C-terminus of a recombinant protein. These tags help in protein purification. Additionally, some affinity tags also serve a dual purpose as solubility enhancers for challenging protein targets. Non-limiting examples of affinity tags include FLAG, HA, V5, Myc, Step, His (e.g., 6His-Tag), GST, MBP, SUMO, CBP, Halo®, Nus A, AviTag, streptavidin-tag, and FATT.

In certain embodiments, a comPACT2.0 polynucleotide composition may further comprise at least one sequence that encodes for an affinity tag or peptide. In certain embodiments, the comPACT2.0 polynucleotide comprises at least two affinity tags or peptide sequences.

Any appropriate affinity tag or peptide may be used in a comPACT polynucleotide or polypeptide. In certain embodiments, the affinity tag or peptide includes, but is not limited to, AviTag, streptavidin-tag, polyhistidine (His6)-tag, FLAG-tag, HA-tag, and Myc-tag.

### 1.6. Protease Cleavage Sites

In certain embodiments, the presently disclosed polynucleotides comprise a sequence encoding a protease cleavage site. The use of protease cleavage sites allows for the removal of affinity tags and the purification of the comPACT2.0 polypeptide by enzymatic reaction of a protease. Non-limiting examples of protease include enteropeptidase, thrombin, factor Xa, TEV protease, and rhinovirus 3C protease.

In certain embodiments, comPACT2.0 polynucleotide composition may further comprise a sequence that encodes for a protease cleavage site, e.g., in the purification cluster. In certain embodiments, the cleavage site may be encoded between the first and second affinity tag sequences and allow for cleavage of the second affinity tag from the comPACT2.0 polypeptide once the comPACT2.0 has been expressed and undergone a round of purification. Any appropriate protease cleavage site known in the art may be used.

### 1.7. PolyA Tail

In certain embodiments, the presently disclosed polynucleotides comprise a polyA tail. The polyA tail comprises multiple adenosine monophosphates. A polyA tail is added to an RNA at the end of transcription. On mRNAs, the polyA tail protects the mRNA molecule from enzymatic degradation in the cytoplasm and aids in transcription termination and translation. In certain non-limiting embodiments, the polyA tail can be a simian virus 40 (SV40) polyA tail, an SV40 polyA tail, a human growth hormone (hGH) polyA tail, a bovine growth hormone (BGH) polyA tail, or a rabbit beta-globin (rbGlob) polyA tail.

In certain embodiments, a comPACT2.0 polynucleotide composition may further comprise a polyadenylation (polyA) tail. In certain embodiments, the polyA tail may be a mammalian motif, eukaryotic motif, or prokaryotic polyA sequence motif.

### 1.9. Biotinylation

In certain embodiments, the comPACT2.0 polypeptides described herein may further be biotinylated.

### 3. Expression Constructs and Vectors

In certain embodiments, the present disclosure provides expression constructions and/or vectors comprising the presently disclosed polynucleotides.

In certain embodiments, the vector can be a viral vector or a non-viral vector. Non-limiting examples of viral vectors include lentiviral, retroviral, adenoviral, herpes virus, and adeno-associated viruses known in the art. Non-limiting examples of non-viral vectors include plasmids, transposon-modified polynucleotides (such as the MVM intron), lipoplexes, polymersomes, polyplexes, dendrimers, inorganic nanoparticles, cell-penetrating peptides and combinations thereof. In certain embodiments, the non-viral vector acts by passive permeabilization of the plasma membrane. Non-limiting examples of non-viral vectors acting by passive permeabilization include peptides, streptolysin O, and cationic derivatives of polyene antibiotics.

In certain embodiments, the comPACT2.0 polynucleotide molecules are inserted into expression constructs or vectors, e.g., for plasmid and protein production. In certain embodiments, the expression construct or vector is a plasmid or a viral vector. In certain embodiments, any suitable expression construct or vector known in the art may be used, including bacterial expression plasmids, such as Escherichia coli or Bacillus subtilis plasmids; eukaryotic expression vectors, such as Pichia pastoris expression vectors; or viral vectors, such as lentiviral vectors, vaccinia vectors, or baculovirus vectors. In certain embodiments, mammalian expression vectors for use in cultured mammalian cell lines such as Chinese hamster ovary (CHO), HEK293, Expi293, or any other suitable mammalian cell line are also contemplated. In certain embodiments, the expression construct or vector may further comprise a nucleotide barcode. In certain embodiments, the nucleotide barcode is unique for each expression construct or vector.

In certain embodiments, the nucleotide sequences encoding for the signal sequence, beta-2-microglobulin, and MHC allele is ligated into an expression construct or vector with a non-coding or dummy antigen insert (e.g., the sequence insert described in Section 1.1 above). In certain embodiments, the non-coding antigen insert can then be removed by an appropriate cloning technique, such as restriction digest, and the desired antigen sequence inserted via ligation or any other appropriate cloning technique.

In certain embodiments, the nucleotide sequences encoding for the signal sequence, beta-2-microglobulin, and MHC allele is ligated into an expression construct or vector with a sequence insert (e.g., described in Section 1.1 above). In certain embodiments, the sequence insert can then be removed by an appropriate cloning technique, such as restriction digest, and the desired antigen sequence inserted via ligation or any other appropriate cloning technique.

In certain embodiments, provided herein are libraries comprising two or more distinct vectors encoding two or more different comPACT2.0 polypeptides. In certain embodiments, each of the two or more different comPACT2.0 polypeptides encodes a different MHC allele.

### 4. Host Cells

In certain embodiments, provided herein are host cells comprising the polynucleotide molecule or the expression construct as described herein.

As used herein, the term “host cell” refers to a cell that has been genetically modified to include a polynucleotide disclosed herein (e.g., one disclosed in Section 1). A host cell can produce a comPACT2.0 polypeptide (e.g., a peptide bound comPACT2.0 polypeptide or an empty comPACT2.0 polypeptide).

In certain embodiments, the host cell is any suitable host cell known in the art, including, but not limited to bacterial cells such as Escherichia coli or Bacillus subtilis, or eukaryotic host cells such as Chinese hamster ovary (CHO), HEK293, Expi293, HeLa, insect cell lines such as Sf9 or Sf12, or yeast cells such as Pichia pastoris. In certain embodiments, the host cells may also stably express the biotinylating enzyme BirA.

### 5. Libraries

In certain embodiments, two or more different comPACT2.0 polynucleotides are combined to make a comPACT2.0 library. In certain embodiments, the library comprises 10 to 1000 comPACT2.0 polynucleotides. In certain embodiments, each of the 10 to 1000 comPACT2.0 polynucleotides express a different HLA allele. In certain embodiments, the library comprises between 2-900, 2-800, 2-700, 2-600, 2-500, 2-480, 2-400, 2-300, 2-200, 2-100, 2-50, 2-66, 2-48, 2-30, 2-20, 2-19, 10-1000, 10-900, 10-800, 10-700, 10-600, 10-500, 10-480, 10-400, 10-300, 10-200, 10-100, 10-50, 10-66, 10-48, 10-30, 10-20, 20-1000, 20-900, 20-800, 20-700, 20-600, 20-500, 20-480, 20-400, 20-300, 20-200, 20-100, 20-50, 20-50, 20-66, 20-48, 20-30, 30-1000, 30-900, 30-800, 30-700, 30-600, 30-500, 30-480, 30-400, 30-300, 30-200, 30-100, 30-50, 30-50, 30-66, 30-48, 30-40, 40-1000, 40-900, 40-800, 40-700, 40-600, 40-500, 40-480, 40-400, 40-300, 40-200, 40-100, 40-60, 40-50, 40-66, 40-48, 50-1000, 50-900, 50-800, 50-700, 50-600, 50-500, 50-480, 50-400, 50-300, 50-200, 50-100, 50-60, 50-66, 60-1000, 60-900, 60-800, 60-700, 60-600, 60-500, 60-480, 60-400, 60-300, 60-200, 60-100, 70-1000, 70-900, 70-800, 70-700, 70-600, 70-500, 70-480, 70-400, 70-300, 70-200, 70-100, 70-80, 70-90, 80-1000, 80-900, 80-800, 80-700, 80-600, 80-500, 80-480, 80-400, 80-300, 80-200, 80-100 comPACT2.0 different polynucleotides, each with a different HLA allele. In certain embodiments, the library comprises between 2-50, between 10-200, between 50-100, between 50-200, between 50-300, or between 50-400 comPACT2.0 different polynucleotides, each with a different HLA allele. In certain embodiments, the library comprises at least 2, 5, 10, 15, 20, 25, 30, 35, 40, 45, 48, 50, 55, 60, 65, 66, 70, 75, 80, 85, 90, 100, 110, 120, 130, 140, 150, 160, 170, 180, 190, 200, 225, 250, 275, 300, 325, 350, 375, 400, 425, 450, 475, 500, 525, 550, 600, 562, 650, 675, 700, 725, 750, 775, 800, 825, 850, 875, 900, 925, 950, 975, or 1000 comPACT2.0 different polynucleotides, each with a different HLA allele. In some embodiments, the library comprises 2, 10, 15, 20, 24, 48, 66, 100, 200, 300, 400, 500, 600, 700, 800, 900, or 1000 comPACT2.0 different polynucleotides, each with a different HLA allele.

In some embodiments, the library comprises greater than or equal to two distinct polynucleotide molecules, wherein each distinct polynucleotide molecule comprises (i) the first universal sequence, (ii) the nucleotide sequence encoding an antigenic peptide, wherein the nucleotide sequence is not the same for each of the greater than or equal to two polynucleotide molecules (iii) the second universal target sequence, (iv) the β2M sequence, and (v) the MHC allele sequence. In some embodiments, the MHC allele sequence is not the same for each of the greater than or equal to two polynucleotide molecules.

In certain embodiments, two or more different comPACT2.0 polypeptides are combined to make a comPACT2.0 library. In certain embodiments, the library comprises 10 to 1000 comPACT2.0 polypeptides. In certain embodiments, each of the 10 to 1000 comPACT2.0 polypeptides express a different HLA allele. In certain embodiments, the library comprises between 2-900, 2-800, 2-700, 2-600, 2-500, 2-480, 2-400, 2-300, 2-200, 2-100, 2-50, 2-66, 2-48, 2-30, 2-20, 2-19, 10-1000, 10-900, 10-800, 10-700, 10-600, 10-500, 10-480, 10-400, 10-300, 10-200, 10-100, 10-50, 10-66, 10-48, 10-30, 10-20, 20-1000, 20-900, 20-800, 20-700, 20-600, 20-500, 20-480, 20-400, 20-300, 20-200, 20-100, 20-50, 20-50, 20-66, 20-48, 20-30, 30-1000, 30-900, 30-800, 30-700, 30-600, 30-500, 30-480, 30-400, 30-300, 30-200, 30-100, 30-50, 30-50, 30-66, 30-48, 30-40, 40-1000, 40-900, 40-800, 40-700, 40-600, 40-500, 40-480, 40-400, 40-300, 40-200, 40-100, 40-60, 40-50, 40-66, 40-48, 50-1000, 50-900, 50-800, 50-700, 50-600, 50-500, 50-480, 50-400, 50-300, 50-200, 50-100, 50-60, 50-66, 60-1000, 60-900, 60-800, 60-700, 60-600, 60-500, 60-480, 60-400, 60-300, 60-200, 60-100, 70-1000, 70-900, 70-800, 70-700, 70-600, 70-500, 70-480, 70-400, 70-300, 70-200, 70-100, 70-80, 70-90, 80-1000, 80-900, 80-800, 80-700, 80-600, 80-500, 80-480, 80-400, 80-300, 80-200, 80-100 comPACT2.0 different polypeptides, each with a different HLA allele. In certain embodiments, the library comprises between 2-50, between 10-200, between 50-100, between 50-200, between 50-300, or between 50-400 comPACT2.0 different polypeptides, each with a different HLA allele. In certain embodiments, the library comprises at least 2, 5, 10, 15, 20, 25, 30, 35, 40, 45, 48, 50, 55, 60, 65, 66, 70, 75, 80, 85, 90, 100, 110, 120, 130, 140, 150, 160, 170, 180, 190, 200, 225, 250, 275, 300, 325, 350, 375, 400, 425, 450, 475, 500, 525, 550, 600, 562, 650, 675, 700, 725, 750, 775, 800, 825, 850, 875, 900, 925, 950, 975, or 1000 comPACT2.0 different polypeptides, each with a different HLA allele. In some embodiments, the library comprises 2, 10, 15, 20, 24, 48, 66, 100, 200, 300, 400, 500, 600, 700, 800, 900, or 1000 comPACT2.0 different polypeptides, each with a different HLA allele.

In certain embodiments, the polynucleotide or polypeptide comPACT2.0 library comprises at least two or more of the HLA-A*01:01, HLA-A*02:01, HLA-A*03:01, HLA-A*24:02, HLA-A*30:02, HLA-A*31:01, HLA-A*32:01, HLA-A*33:01, HLA-A*68:01, HLA-A*11:01, HLA-A*23:01, HLA-A*30:01, HLA-A*33:03, HLA-A*25:01, HLA-A*26:01, HLA-A*29:02, HLA-A*68:02, HLA-B*07:02, HLA-B*14:02, HLA-B*18:01, HLA-B*27:02, HLA-B*39:01, HLA-B*40:01, HLA-B*44:02, HLA-B*46:01, HLA-B*50:01, HLA-B*57:01, HLA-B*58:01, HLA-B*08:01, HLA-B*15:01, HLA-B*15:03, HLA-B*35:01, HLA-B*40:02, HLA-B*42:01, HLA-B*44:03, HLA-B*51:01, HLA-B*53:01, HLA-B*13:02, HLA-B*15:07, HLA-B*27:05, HLA-B*35:03, HLA-B*37:01, HLA-B*38:01, HLA-B*41:02, HLA-B*44:05, HLA-B*49:01, HLA-B*52:01, HLA-B*55:01, HLA-C*02:02, HLA-C*03:04, HLA-C*05:01, HLA-C*07:01, HLA-C*01:02, HLA-C*04:01, HLA-C*06:02, HLA-C*07:02, HLA-C*16:01, HLA-C*03:03, HLA-C*07:04, HLA-C*08:01, HLA-C*08:02, HLA-C*12:02, HLA-C*12:03, HLA-C*14:02, HLA-C*15:02, and HLA-C*17:01 alleles. In one embodiment, the library comprises at least HLA-A*01:01, HLA-A*02:01, HLA-A*03:01, HLA-A*24:02, HLA-A*30:02, HLA-A*31:01, HLA-A*32:01, HLA-A*33:01, HLA-A*68:01, HLA-A*11:01, HLA-A*23:01, HLA-A*30:01, HLA-A*33:03, HLA-A*25:01, HLA-A*26:01, HLA-A*29:02, HLA-A*68:02, HLA-B*07:02, HLA-B*14:02, HLA-B*18:01, HLA-B*27:02, HLA-B*39:01, HLA-B*40:01, HLA-B*44:02, HLA-B*46:01, HLA-B*50:01, HLA-B*57:01, HLA-B*58:01, HLA-B*08:01, HLA-B*15:01, HLA-B*15:03, HLA-B*35:01, HLA-B*40:02, HLA-B*42:01, HLA-B*44:03, HLA-B*51:01, HLA-B*53:01, HLA-B*13:02, HLA-B*15:07, HLA-B*27:05, HLA-B*35:03, HLA-B*37:01, HLA-B*38:01, HLA-B*41:02, HLA-B*44:05, HLA-B*49:01, HLA-B*52:01, HLA-B*55:01, HLA-C*02:02, HLA-C*03:04, HLA-C*05:01, HLA-C*07:01, HLA-C*01:02, HLA-C*04:01, HLA-C*06:02, HLA-C*07:02, HLA-C*16:01, HLA-C*03:03, HLA-C*07:04, HLA-C*08:01, HLA-C*08:02, HLA-C*12:02, HLA-C*12:03, HLA-C*14:02, HLA-C*15:02, and HLA-C*17:01 alleles.

In certain embodiments, the polynucleotide or polypeptide comPACT2.0 library comprises at least an HLA-B44 allele. In certain embodiments the HLA-B44 allele is B44:02. In certain embodiments the HLB-A44 allele is B44:03.

In certain embodiments, the polynucleotide or polypeptide comPACT2.0 library comprises at least an HLA-A33 allele. In certain embodiments the HLA-A33 allele is A33:03. In certain embodiments the HLA-A33 allele is A33:01.

In certain embodiments, the polynucleotide or polypeptide comPACT2.0 library comprises at least an HLA-A33 allele and an HLA-B44 allele. In certain embodiments the HLA-A33 allele is A33:03 or A33:01 and the HLA-B44 allele is B44:02 or B44:03. In certain embodiments the HLA-A33 allele is A33:03 and the HLA-B44 allele is B44:02. In certain embodiments the HLA-A33 allele is A33:03 and the HLA-B44 allele is B44:03. In certain embodiments the HLA-A33 allele is A33:03 and the HLA-B44 allele is B44:02 and B44:03. In certain embodiments the HLA-A33 allele is A33:01 and the HLA-B44 allele is B44:02. In certain embodiments the HLA-A33 allele is A33:01 and the HLA-B44 allele is B44:03. In certain embodiments the HLA-A33 allele is A33:01 and the HLA-B44 allele is B44:02 and B44:03.

In certain embodiments, the library comprises greater than or equal to two distinct polypeptide molecules, wherein the antigenic peptide is not the same for each of the greater than or equal to two polypeptide molecules, and wherein each distinct polypeptide is attached to a particle. In certain embodiments, the library further comprises a unique defined barcode sequence operably associated with the identity of each distinct polypeptide.

In certain embodiments, each comPACT2.0 polypeptide in the comPACT2.0 library is barcoded. In certain embodiments, the barcode is a polynucleotide that provides a unique antigen-specific sequence for identification after T cell isolation. In certain embodiments, each comPACT2.0 polypeptide comprises a unique defined barcode sequence. In certain embodiments, the barcode provides an operative association between a given antigen and a given barcode that is unique to the pair.

In certain embodiments, the barcode is ssDNA or dsDNA. In certain embodiments, the polynucleotides comprising the barcode are modified at its 5′ end to comprise an attachment moiety for attachment to a particle. In certain embodiments, the polynucleotides comprising the barcode are conjugated to a biotin molecule for binding to a streptavidin-core attached to a particle, such as dextran. In certain embodiments, other suitable attachment moieties can be used for the attachment of polynucleotides to a particle. In certain embodiments, non-limiting examples of attachment moieties include thiol, maleimide, adamantane, cyclodextrin, amine, carboxy, azide, and alkyne.

### 6. Particles

As used herein, “nanoparticles” or alternatively “particles” refer to substrates capable of being specifically sorted or isolated, and to which other entities can be attached. In certain embodiments, the nanoparticle is magnetic, e.g., for isolation using a magnet. In certain embodiments, the magnetic nanoparticle comprises magnetic iron oxide. In certain embodiments, the nanoparticle is a polystyrene particle, e.g., for isolation by gravity. In certain embodiments, the particle is a surface, a bead, or a polymer. In certain embodiments, the particle or nanoparticle is fluorescent or is attached to a fluorophore directly or indirectly.

In certain embodiments, the nanoparticle is modified with an attachment moiety for attaching additional molecules. In certain embodiments, modification of the nanoparticle includes an attachment moiety that can pair with (e.g., covalently bind to) a corresponding cognate (e.g., complementary) attachment moiety attached to polynucleotides. In certain embodiments, any suitable pair of attachment moieties may be used to modify the nanoparticle and the polynucleotide detection tag for attachment including but not limited to a streptavidin/biotin system, a thiol group (e.g., cysteine), and maleimide, adamantane and cyclodextrin, an amino group and a carboxy group, and an azido group and alkynl group. In certain embodiments, the attachment moiety comprises a cleavage moiety. In certain embodiments, the attachment moiety bound to complementary cognate attachment moiety is reversible, such as a reducible thiol group. In certain embodiments, the modified nanoparticle is a streptavidin coated magnetic nanoparticle, such as 1 μm nanoparticles (e.g., Dynabeads™ MyOne™ Streptavidin T1 beads from ThermoFisher Scientific), and the polynucleotides can be biotinylated for attachment to the modified nanoparticle.

In certain embodiments, the particle is a dextran, such as a biotinylated dextran or streptavidin coated dextran. In certain embodiments, the dextran is a modified dextran. In certain embodiments, biotinylated comPACT2.0 polypeptides can be attached to streptavidin coated dextran.

In certain embodiments, comPACT2.0 polypeptides are assembled into tetramers, comprising 1, 2, 3, or 4 biotinylated comPACT2.0 polypeptides bound to a streptavidin core. In certain embodiments, the tetramer further comprises a fluorophore. In certain embodiments, the fluorophore is phycoerythrin (PE) or allophycocyanin (APC).

In certain embodiments, comPACT2.0 polypeptides are assembled into multimers. In certain embodiments, the comPACT2.0 polypeptide multimer is a dimer, trimer, tetramer, pentamer, hexamer, or higher order multimer. In certain embodiments, a multimer comprises at least two or more comPACT2.0 polypeptides. In certain embodiments, a multimer comprises at least 2, 3, 4, 5, 6, 7, 8, 9, or 10 comPACT2.0 polypeptides.

### 7. Methods of Producing

### 7.1. Antigen Prediction

In certain embodiments, the initial step in the manufacture of a comPACT2.0 polypeptide includes the identification of the patient's tumor-specific antigens (e.g., private neoantigens). In certain embodiments, the compositions produced by this method are then utilized in a T-cell mediated immunity process, e.g., for patient-specific cancer immunotherapy. In certain embodiments, in silico predictive algorithmic programs are used to identify a patient's putative neoantigens (tumor or pathogen). In certain embodiments, the algorithms are utilized to analyze the tumor, viral, or bacterial sequencing data including whole genome, whole exome, or transcriptome sequencing data, to identify one or more mutations corresponding to putatively expressed neoantigens. In certain embodiments, human leukocyte antigen (HLA) typing is determined from a tumor or blood sample of the patient, and this HLA information is utilized together with the identified putative neoantigen peptide sequences in a predictive algorithm for MHC binding, as verified by Fritsch et al., 2014, Cancer Immunol Res., 2:522-529, the entirety of which is herein incorporated by reference. In certain embodiments, HLAs commonly found in the human population can also be included in neoantigen prediction algorithms, such as HLA-A*02, 24, 01; HLA-B*35, 44, 51; DRB1*11, 13, 07 in Caucasians, HLA-A*02, 03, 30; HLA-B*35, 15, 44; DRB1*13, 11, 03 in afro-Brazilians, and HLA-A*24, 02, 26; HLA-B*40, 51, 52; DRB1*04, 15, 09 in Asians. In certain embodiments, the specific pairing of HLA alleles can also be used.

### 7.2. Primer Annealing and Restriction Digest Assembly

In certain embodiments, preparation of a comPACT2.0 polynucleotide (e.g., encoding a peptide bound comPACT2.0 polynucleotide) is accomplished by procedures disclosed herein and by recognized recombinant DNA techniques, e.g., preparation of plasmid DNA, cleavage of DNA with restriction enzymes, ligation of DNA, transformation or transfection of a host, culturing of the host, and isolation and purification of the expressed fusion complex.

In certain embodiments, DNA encoding an MHC class I heavy chain is obtained from a suitable cell line such as, for example, human lymphoblastoid cells. In certain embodiments, a gene or cDNA encoding a class I heavy chain is amplified by the polymerase chain reaction (PCR) or other means known in the art. In certain embodiments, a PCR product includes sequences encoding linkers, and/or one or more restriction enzyme sites for ligation of such sequences.

In certain embodiments, a vector comprising a comPACT2.0 polynucleotide (e.g., encoding a peptide bound comPACT2.0 polynucleotide) is prepared by ligation of sequences encoding the MHC heavy chain, and the β2-microglobulin to a sequence encoding an antigen peptide. In certain embodiments, DNA encoding the antigen peptide is obtained by isolating DNA from natural sources or by known synthetic methods. In certain embodiments, synthetic oligonucleotides are prepared using commercially available automated oligonucleotide synthesizers. In certain embodiments, a DNA sequence encoding a universal target sequence as disclosed herein is interposed between a sequence encoding a signal sequence and a sequence encoding an antigenic peptide and a second universal target sequence can be interposed between the sequence encoding an antigen peptide segment and a sequence encoding a β2-microglobulin segment. In certain embodiments, the segments are joined using a ligase. In certain embodiments, the sequence encoding an antigen peptide is phosphorylated with a suitable polynucleotide kinase. In some embodiments, the polynucleotide kinase is the T4 polynucleotide kinase.

In certain embodiments, a vector comprising a comPACT2.0 polynucleotide (e.g., encoding an empty comPACT2.0 polynucleotide) is prepared by ligation of sequences encoding the MHC heavy chain and the β2-microglobulin to a sequence encoding linker. In certain embodiments, the linker comprises the amino acid sequence set forth in SEQ ID NO: 133 or SEQ ID NO: 134. In certain embodiments, the linker consists of the amino acid sequence set forth in SEQ ID NO: 133 or SEQ ID NO: 134.

### 7.3. PCR Assembly

In certain embodiments, the comPACT2.0 polynucleotide (e.g., encoding a peptide bound comPACT2.0 polynucleotide) is assembled via polymerase chain reaction (PCR) amplification. In certain embodiments, DNA encoding the MHC heavy chain and the β2-microglobulin are obtained from a suitable source. In certain embodiments, a second DNA fragment encoding a chosen signal sequence is obtained from a suitable source. In certain embodiments, both fragments of DNA may have different universal target sequences, such that primers for one universal sequence do not anneal to the second universal sequence. In certain embodiments, two sequences encoding an antigenic peptide are synthesized; one forward primer with the antigenic sequence at the 5′ end and the complement of the universal primer sequence on the MHC DNA fragment at the 3′ end; and one reverse primer with the reverse complement of the chosen antigenic sequence at the 5′ end and the reverse complement of the universal primer from the signal sequence fragment at the 3′ end. In certain embodiments, a PCR reaction with all four DNA fragments and primer for the 5′ end of the signal sequence fragment and 3′ end of the MHC allele fragment results in the amplification of two DNA fragments, one with the signal sequence at the 3′ end and the antigenic sequence at the 5′ end, and one with the antigenic sequence at the 3′ end and the MHC allele at the 3′ end. In certain embodiments, a further PCR amplification cycle is used to allow the overlapping antigenic peptide sequences to anneal and result in a single full-length DNA fragment. In certain embodiments, the signal peptide fragment further comprises a promoter sequence. In certain embodiments, the MHC fragment further comprises a purification cluster and/or a polyA tail.

### 7.4. Transfection, Transduction, and Genetic Modification of Host Cells

In certain embodiments, a comPACT2.0 polynucleotide is inserted into the host cell via an appropriate method known, including, but not limited to, transfection, transduction, electroporation, lipofection, sonoporation, mechanical disruption, or viral vectors. In certain embodiments, a comPACT2.0 polynucleotide is inserted by non-viral transfection.

In certain embodiments, a comPACT2.0 polynucleotide is transiently or stably expressed in the host cell. In certain embodiments, the comPACT2.0 polynucleotide is integrated into the host genome. In certain embodiments, the comPACT polynucleotide is extra-chromosomal.

In certain embodiments, any appropriate genetic editing technique known in the art may also be employed to modify the host cell with the comPACT polynucleotide, including CRISPR/Cas9, zinc-finger nucleases, or TALEN nucleases.

### 7.5. Expression

In certain embodiments, a comPACT2.0 polypeptide is expressed in a cell. In certain embodiments, the comPACT2.0 polypeptide is incorporated into a suitable vector by known methods such as by use of restriction enzymes and ligases. In certain embodiments, a vector is selected based on factors relating to the cloning protocol. In certain embodiments, the vector is compatible with and has the proper replicon for the host that is being employed. In certain embodiments, suitable host cells include eukaryotic and prokaryotic cells. In certain embodiments, the host cells are easily transfected and exhibit rapid growth in the culture medium. In certain embodiments, the host cells are prokaryotic cells. In certain embodiments, the prokaryotic cells are E. coli or B. subtilis. In certain embodiments, the host cells are eukaryotic cells. In certain embodiments, the eukaryotic cells are animal cells or yeast cells. In certain embodiments, the animal cells are mammalian cells and/or human cells. In certain embodiments, the mammalian cells are J558, NSO, SP2-O, 293T, Expi293, and CHO. In certain embodiments, the cells are insect cells.

In certain embodiments, the manufacture of a comPACT2.0 polypeptide yields a product that is substantially-free of LPS. In certain embodiments, the manufacture of a comPACT2.0 polypeptide yields glycosylated comPACT2.0 polypeptides. In certain embodiments, the manufacture of a comPACT2.0 polypeptide yields a comPACT2.0 that includes one or more post-translational modifications. In certain embodiments, the manufacture of a comPACT2.0 polypeptide yields a comPACT2.0 polypeptide that is substantially free of LPS or free of LPS and is glycosylated.

In certain embodiments, the presently disclosed comPACT2.0 polypeptides include a disulfide bond within the MHC heavy chain sequence. In certain embodiments, the disulfide bond is between the amino acid residue 84 and the amino acid 139 of the MHC heavy chain sequence.

### 7.6. Purification (Chromatography)

In certain embodiments, a comPACT2.0 polypeptide is isolated and purified by known methods. In certain embodiments, a comPACT2.0 polypeptide comprising a His6 affinity tag may is purified via affinity chromatography on a Ni-NTA column by procedures that are generally known and disclosed. In certain embodiments, a comPACT2.0 polypeptide comprising human HLA sequences is purified by affinity chromatography on a monoclonal antibody-Sepharose column by procedures that are generally known and disclosed.

### 7.7. Peptide Loading

In certain embodiments, the present disclosure provides methods of preparing a peptide loaded comPACT2.0 polypeptide. In certain embodiments, a first polypeptide comprising an antigenic peptide (e.g., a private neoantigen) is incubated with a second polypeptide comprising a β2M sequence, and an MHC allele sequence (e.g., an empty comPACT2.0 polypeptide). In certain embodiments, after incubation, the antigenic peptide non-covalently binds to the empty comPACT2.0 polypeptide in order to obtain a peptide loaded comPACT2.0 polypeptide. In certain embodiments, the incubation occurs at conditions sufficient for the antigenic peptide to bind the empty comPACT2.0 polypeptide.

### 8. T Cell Isolation

In certain embodiments, provided herein are methods of isolating an antigen-specific T cell, the method comprising the steps of (a) providing a peptide bound comPACT2.0 polypeptide wherein the peptide bound comPACT2.0 polypeptide is linked to one particle; (b) providing a sample known or suspected to comprise one or more T cells; (c) contacting the peptide bound comPACT2.0 polypeptide with the sample, wherein the contacting comprises providing conditions sufficient for a single T cell to bind the peptide bound comPACT2.0 polypeptide attached to the particle, and (d) isolating the single T cell associated with the particle.

In certain embodiments, provided herein are methods of isolating an antigen-specific T cell, the method comprising the steps of (a) providing a peptide loaded comPACT2.0 polypeptide, wherein the peptide loaded comPACT2.0 polypeptide is linked to one particle; (b) providing a sample known or suspected to comprise one or more T cells; (c) contacting the peptide loaded comPACT2.0 polypeptide with the sample, wherein the contacting comprises providing conditions sufficient for a single T cell to bind the peptide loaded comPACT2.0 polypeptide attached to the particle, and (d) isolating the single T cell associated with the particle.

In certain embodiments, provided herein are methods of isolating an antigen-specific T cell, the method comprising the steps of (a) providing a polypeptide comprising, in an amino terminus to carboxyl terminus orientation, (i) a first universal target peptide, (ii) an antigenic peptide, (iii) a second universal target peptide that is distinct from the first universal target peptide, (iv) a β2M peptide, and (v) an MHC peptide, wherein the polypeptide is linked to one particle; (b) providing a sample known or suspected to comprise one or more T cells; (c) contacting the polypeptide with the sample, wherein the contacting comprises providing conditions sufficient for a single T cell to bind the polypeptide attached to the particle, and (d) isolating the single T cell associated with the particle.

In certain embodiments, isolation and identification of patient-derived and antigen-specific T cells using a comPACT2.0 polypeptide as described herein includes incubating the comPACT2.0 polypeptide with patient-derived T cells. In certain embodiments, the comPACT2.0 polypeptide library is incubated with patient-derived T cells. In certain embodiments, the T cells are derived from a tissue such as blood, a lymph node, or a tumor.

In certain embodiments, the patient-derived T cells are isolated from a patient's peripheral blood mononuclear cells (PBMCs) or tumor-infiltrating lymphocytes (TILs). In certain embodiments, both CD4+ and CD8+ T cells are labeled and sorted from PBMCs or TILS using anti-CD4 and anti-CD8 fluorescent antibodies, with live populations of CD4+ and CD8+ single-positive cells sorted using fluorescence-activated cell sorting (FACS), to isolate only CD4+ or CD8+ cells. In certain embodiments, T cells that are positive for both CD4 and CD8 are isolated using an anti-CD3 fluorescent antibody followed by FACS.

In certain embodiments, incubation of the comPACT2.0 polypeptide or comPACT2.0 polypeptide library with the T cell suspension allows for complete and thorough exposure of the particle-bound antigen to the various T-cell receptors. In certain embodiments, this method includes rocking or rotation of the cells. In certain embodiments, the comPACT2.0 polypeptide is associated with a particle.

In certain embodiments, following incubation of the comPACT2.0 polypeptide or comPACT2.0 polypeptide library and the T cells, the bound comPACT2.0-T cell complex is selectively separated or selectively collected. In certain embodiments, T cells are bound to many identical copies of identical comPACT2.0 polypeptides within the comPACT2.0 library and are separated based on these interactions. In certain embodiments, if the comPACT2.0 polypeptide comprises a fluorophore, or is attached to a particle with a fluorophore, fluorescent associated cell sorting (FACS), including single-cell sorting, is used to selectively isolate the T cells. In certain embodiments, if the comPACT2.0 polypeptide is attached to a magnetic particle, applying a magnet to the suspension can allow for the separation of particles complexed with antigen-paired T cells and the removal of unpaired T cells. In certain embodiments, if the particle is a polystyrene particle, the unpaired T cells are separated by gravity (e.g., centrifugation). In certain embodiments, following the removal of unpaired T cells, the separated bound particles are washed at least once to remove any non-specifically associated T cells.

In certain embodiments, comPACT2.0-bound T cells are separated by FACS into individual collection containers, such as a multi-well plate. In certain embodiments, the individual collection container is a single-cell reaction vessel. In certain embodiments, the comPACT-bound T cells are separated by FACS into a bulk collection container (e.g., every T cell isolated is collected in the same container).

In certain embodiments, comPACT2.0-bound T cells are individually isolated in droplets using a droplet generating microfluidic device.

In certain embodiments, following the isolation of comPACT2.0-bound T cells into single-cell reaction vessels (e.g., isolated in individual wells or droplets), the nucleic acid of the comPACT-bound T cell is further processed for downstream analysis. In certain embodiments, the expressed TCRα and TCRβ mRNA transcripts are first converted to cDNA by reverse transcription, and the cDNA amplified for next-generation sequencing (NGS) methods known to those skilled in the art.

### 9. Exemplary Embodiments

A1. In certain non-limiting embodiments, the present disclosure provides a polynucleotide comprising, in a 5′ to 3′ orientation, a sequence insert, a beta 2 Microglobulin (β2M) sequence, and a Major Histocompatibility Complex (MHC) allele sequence, wherein the sequence insert comprises a stop codon in each open reading frame, and wherein the MHC allele sequence comprises a Y84C mutation and a A139C mutation.

A2. The foregoing polynucleotide of A1, wherein the sequence insert is flanked by a first universal target sequence.

A3. The foregoing polynucleotide of A1 or A2, wherein the sequence insert is flanked by a second universal target sequence.

A4. In certain non-limiting embodiments, the present disclosure provides a polynucleotide comprising, in a 5′ to 3′ orientation, a first universal target sequence, a nucleotide sequence encoding an antigenic peptide, a second universal target sequence, a Beta 2 Microglobulin (β2M) sequence, and a Major Histocompatibility Complex (MHC) allele sequence, wherein the MHC allele sequence comprises a Y84C mutation and a A139C mutation.

A5. The foregoing polynucleotide of A1 or A4 further comprising a linker positioned 5′ end of the β2M sequence.

A6. The foregoing polynucleotide of A5, wherein the linker comprises the amino acid sequence set forth in SEQ ID NO: 133 or SEQ ID NO: 134.

A7. In certain non-limiting embodiments, the present disclosure provides a polynucleotide comprising, in a 5′ to 3′ orientation, a sequence encoding a linker, a Beta 2 Microglobulin (β2M) sequence, and a Major Histocompatibility Complex (MHC) allele sequence, wherein the MHC allele sequence comprises a Y84C mutation and a A139C mutation.

A8. The foregoing polynucleotide of A7, wherein the linker comprises the amino acid sequence set forth in SEQ ID NO: 133 or SEQ ID NO: 134.

A9. The foregoing polynucleotide of any one of A1-A6, wherein the antigenic peptide is a neoantigen.

A10. The foregoing polynucleotide of A9, wherein the antigenic peptide is a public neoantigen.

All. The foregoing polynucleotide of A10, wherein the public neoantigen is a HPV neoantigen.

A12. The foregoing polynucleotide of A11, wherein the HPV neoantigen is derived from E16 protein or E17 protein.

A13. The foregoing polynucleotide of A9, wherein the antigenic peptide is a private neoantigen.

A14. The foregoing polynucleotide of any one of A1-A13, wherein the β2M allele is a human β2M allele.

A15. The foregoing polynucleotide of any one of A1-A14, wherein the MHC allele is a human MHC allele.

A16. The foregoing polynucleotide of A15, wherein the human MHC allele is an HLA allele selected from the group consisting of: HLA-A*01:01, HLA-A*02:01, HLA-A*03:01, HLA-A*24:02, HLA-A*30:02, HLA-A*31:01, HLA-A*32:01, HLA-A*33:01, HLA-A*68:01, HLA-A*11:01, HLA-A*23:01, HLA-A*30:01, HLA-A*33:03, HLA-A*25:01, HLA-A*26:01, HLA-A*29:02, HLA-A*68:02, HLA-B*07:02, HLA-B*14:02, HLA-B*18:01, HLA-B*27:02, HLA-B*39:01, HLA-B*40:01, HLA-B*44:02, HLA-B*46:01, HLA-B*50:01, HLA-B*57:01, HLA-B*58:01, HLA-B*08:01, HLA-B*15:01, HLA-B*15:03, HLA-B*35:01, HLA-B*40:02, HLA-B*42:01, HLA-B*44:03, HLA-B*51:01, HLA-B*53:01, HLA-B*13:02, HLA-B*15:07, HLA-B*27:05, HLA-B*35:03, HLA-B*37:01, HLA-B*38:01, HLA-B*41:02, HLA-B*44:05, HLA-B*49:01, HLA-B*52:01, HLA-B*55:01, HLA-C*02:02, HLA-C*03:04, HLA-C*05:01, HLA-C*07:01, HLA-C*01:02, HLA-C*04:01, HLA-C*06:02, HLA-C*07:02, HLA-C*16:01, HLA-C*03:03, HLA-C*07:04, HLA-C*08:01, HLA-C*08:02, HLA-C*12:02, HLA-C*12:03, HLA-C*14:02, HLA-C*15:02, and HLA-C*17:01.

A17. The foregoing polynucleotide of A16, wherein the HLA allele comprises the amino acid sequence selected from the group consisting of SEQ ID NOs: 1-66.

A18. The foregoing polynucleotide of A16, wherein the HLA allele consists of the amino acid sequence selected from the group consisting of SEQ ID NOs: 1-66.

A19. The foregoing polynucleotide of A16, wherein the HLA allele comprises the amino acid sequence selected from the group consisting of SEQ ID NOs: 137-202.

A20. The foregoing polynucleotide of A16, wherein the HLA allele consists of the amino acid sequence selected from the group consisting of SEQ ID NOs: 137-202.

A21. The foregoing polynucleotide of any one of A15-A20, wherein the human MHC allele is selected from the group consisting of HLA-A*33:01, HLA-A*33:03, HLA-B*44:02, HLA-B*44:03, and HLA-B*44:025.

A22. The foregoing polynucleotide of claim 21, wherein the human MHC allele is HLA-A*33:01.

A23. The foregoing polynucleotide of A21, wherein the human MHC allele is HLA-A*33:03.

A24. The foregoing polynucleotide of A21, wherein the human MHC allele is HLA-B*44:02.

A25. The foregoing polynucleotide of A21, wherein the human MHC allele is HLA-B*44:03.

A26. The foregoing polynucleotide of A21, wherein the human MHC allele is HLA-B*44:025.

A27. The foregoing polynucleotide of any one of A2-A6 and A9-A26, wherein the first and second universal target sequences comprise a polymerase chain reaction (PCR) primer target site.

A28. The foregoing polynucleotide of any one of A2-A6 and A9-A27, wherein the first and second universal target sequences comprise a restriction enzyme cleavage site.

A29. The foregoing polynucleotide of any one of A2-A6 and A9-A28, further comprising a sequence encoding a signal sequence.

A30. The foregoing polynucleotide of any one of A1-A29 further comprising a purification cluster sequence positioned at the 3′ end of the polynucleotide, wherein the purification cluster comprises a first affinity tag sequence, a protease cleavage site sequence, and a second affinity tag sequence.

B. In certain non-limiting embodiments, the present disclosure provides an expression construct comprising the polynucleotide of any one of A1-A30.

C. In certain non-limiting embodiments, the present disclosure provides a vector comprising the polynucleotide of any one of A1-A30.

D. In certain non-limiting embodiments, the present disclosure provides a polypeptide encoded by the polynucleotide of any one of A1-A30.

E. In certain non-limiting embodiments, the present disclosure provides a protein complex comprising a first polypeptide comprising an antigenic peptide and a second polypeptide encoded by the polynucleotide of any one of A7, A8, A14-A26, and A30.

F. In certain non-limiting embodiments, the present disclosure provides a host cell comprising the polynucleotide of any one of A1-A30.

G1. In certain non-limiting embodiments, the present disclosure provides a library comprising at least two polynucleotide of any one of A1-A30, at least two expression constructs of B, at least two vectors of C, at least two polypeptides of D, at least two protein complexes of E, or at least two host cells of F, wherein at least one polynucleotide, at least one expression construct, at least one vector, at least one polypeptide, or at least one cell comprises a human MHC allele selected from the group consisting of HLA-A*33:01, HLA-A*33:03, HLA-B*44:02, HLA-B*44:03, and HLA-B*44:025.

G2. The foregoing library of G1 comprising at least 66 different polynucleotide molecules, 66 different expression constructs, 66 different vectors, 66 different polypeptides.

H. In certain non-limiting embodiments, the present disclosure provides a kit comprising a polynucleotide of any one of A1-A30, an expression construct of B, a vector of C, a polypeptide of D, a protein complex of E, a host cell of F, or a library of G1 or G2 and instructions for use.

I. In certain non-limiting embodiments, the present disclosure provides a method for isolating an antigen-specific T cell, the method comprising: a) providing a plurality of particles, wherein each particle comprises a polypeptide of D or a protein complex of E; b) obtaining a sample known or suspected to comprise one or more T cells; c) contacting the plurality of particles with the sample, wherein the contacting comprises providing conditions sufficient for a single T cell to bind the polypeptide attached to the particle, and d) isolating the single T cell associated with the particle.

J1. In certain non-limiting embodiments, the present disclosure provides a polynucleotide molecule comprising, in a 5′ to 3′ orientation, a sequence insert comprising stop codons in three frames, a Beta 2 Microglobulin (β2M) sequence, and a Multiple Histocompatibility Complex (MHC) allele sequence; wherein the MHC allele sequence is engineered to comprise two cysteine mutations; and wherein the two cysteine mutations are Y84C and a A139C.

J2. The foregoing polynucleotide of J1, wherein the sequence insert is flanked by a first universal target sequence and/or a second universal target sequence.

J3. The foregoing polynucleotide of J1 or J2, further comprising a nucleotide sequence encoding an antigenic peptide.

J4. In certain non-limiting embodiments, the present disclosure provides a polynucleotide molecule comprising, in a 5′ to 3′ orientation, a first universal target sequence, a nucleotide sequence encoding an antigenic peptide, a second universal target sequence that is distinct from the first universal target sequence, a Beta 2 Microglobulin (β2M) sequence, and a Multiple Histocompatibility Complex (MHC) allele sequence wherein the MHC allele sequence is engineered to comprise two cysteine mutations; and wherein the two cysteine mutations are Y84C and a A139C.

J5. The foregoing polynucleotide of any of J1-J4, wherein the linker between the antigenic peptide and β2M is GGGAS(G₄S)₂ (SEQ ID NO:134).

J6. The foregoing polynucleotide of any of J1-J5, wherein the antigenic peptide is a neoantigen.

J7. The foregoing polynucleotide of any one of J1-J6, wherein the MHC allele is a human HLA allele and the β2M allele is a human β2M allele.

J8. The foregoing polynucleotide of J7, wherein the MHC allele is a human the HLA allele is selected from the group consisting of: HLA-A*01:01, HLA-A*02:01, HLA-A*03:01, HLA-A*24:02, HLA-A*30:02, HLA-A*31:01, HLA-A*32:01, HLA-A*33:01, HLA-A*68:01, HLA-A*11:01, HLA-A*23:01, HLA-A*30:01, HLA-A*33:03, HLA-A*25:01, HLA-A*26:01, HLA-A*29:02, HLA-A*68:02, HLA-B*07:02, HLA-B*14:02, HLA-B*18:01, HLA-B*27:02, HLA-B*39:01, HLA-B*40:01, HLA-B*44:02, HLA-B*46:01, HLA-B*50:01, HLA-B*57:01, HLA-B*58:01, HLA-B*08:01, HLA-B*15:01, HLA-B*15:03, HLA-B*35:01, HLA-B*40:02, HLA-B*42:01, HLA-B*44:03, HLA-B*51:01, HLA-B*53:01, HLA-B*13:02, HLA-B*15:07, HLA-B*27:05, HLA-B*35:03, HLA-B*37:01, HLA-B*38:01, HLA-B*41:02, HLA-B*44:05, HLA-B*49:01, HLA-B*52:01, HLA-B*55:01, HLA-C*02:02, HLA-C*03:04, HLA-C*05:01, HLA-C*07:01, HLA-C*01:02, HLA-C*04:01, HLA-C*06:02, HLA-C*07:02, HLA-C*16:01, HLA-C*03:03, HLA-C*07:04, HLA-C*08:01, HLA-C*08:02, HLA-C*12:02, HLA-C*12:03, HLA-C*14:02, HLA-C*15:02, and HLA-C*17:01.

J9. The foregoing polynucleotide of J7, wherein the HLA allele comprises a sequence selected from the group consisting of SEQ ID NOs: 1-132.

J10. The foregoing polynucleotide of any one of J4-J9, wherein the first and second universal target sequences comprise a polymerase chain reaction (PCR) primer target site and/or a restriction enzyme cleavage site.

J11. The foregoing polynucleotide of any one of J4-J10, further comprising a signal sequence.

J12. The foregoing polynucleotide of any one of J1-J11, wherein the 3′ end of the polynucleotide sequence further comprises a purification cluster sequence comprising a first affinity tag sequence, a protease cleavage site sequence, and a second affinity tag sequence.

J13. In certain non-limiting embodiments, the present disclosure provides an expression construct comprising the polynucleotide molecule of any one of J1-J12.

J14. In certain non-limiting embodiments, the present disclosure provides a polypeptide encoded by the polynucleotide of any one of J1-J13.

J15. The polypeptide of J14, wherein the MHC allele encodes for an HLA peptide comprising a Y84A or a Y84C mutation.

J16. In certain non-limiting embodiments, the present disclosure provides a library comprising two polynucleotide molecules of any one of J1-J12, at least two expression constructs of J13, or at least two polypeptides of J14 or J15.

J17. The foreoing library of J16, wherein the library comprises an expression construct that expresses one or more of HLA-A*33:01, HLA-A*33:03, HLA-B*44:02, HLA-B*44:03, and HLA-B*44:025.

J18. The foregoing library of J16 or J17, comprising at least 66 distinct polynucleotide molecules, 66 distinct expression constructs, or 66 distinct polypeptides.

J19. In certain non-limiting embodiments, the present disclosure provides a kit comprising a polynucleotide of any one of J1-J12, an expression construct of J13, a polypeptide of J14 or J15, or a library of J16 or J17 and instructions for use.

J20. In certain non-limiting embodiments, the present disclosure provides a method for isolating and/or identifying an antigen-specific T cell, the method comprising: providing a plurality of particles, wherein each particle comprises a polypeptide of any one of J14 or J15; obtaining a sample known or suspected to comprise one or more T cells; contacting the plurality of particles with the sample, wherein the contacting comprises providing conditions sufficient for a single T cell to bind the polypeptide attached to the particle; and isolating the single T cell associated with the particle.

### EXAMPLES

### Example 1: Design and Cloning of comPACT2.0 Polynucleotides Via Restriction Digest Cloning

Structure of comPACT2.0 Polynucleotides for Restriction Digest:

The basic exemplary components of a comPACT2.0 polynucleotide are the signal sequence that directs the secretion of the protein, universal target sequences such as restriction sites or primer binding sites, the antigenic peptide (or neoantigen, NeoE), a second universal target site, the invariant β2M, the extracellular domain of an MHC allele, and a purification cluster enabling enzymatic modification (e.g. biotinylation) and purification of the comPACT2.0 via affinity tags. The cluster may also contain a protease cleavage site and linker sequences between the peptide components. The comPACT2.0 polynucleotide also contains cysteine mutations that create a disulfide bridge in the translated polynucleotide. The cysteine mutations form the disulfide bridge. A diagram of a comPACT2.0 polynucleotide is shown in FIG. 1. Additional restriction sites upstream and downstream of the MHC heavy chain sequence can be used to insert other MHC alleles to construct different MHC templates and build a library of MHC templates. The DNA fragments encoding the signal sequence, universal target sequences, invariant β2M, and the extracellular domain of an MHC allele are the base MHC template.

For restriction digest cloning methods, each comPACT2.0 DNA construct is a base MHC template with a dummy antigenic sequence insert containing stop codons in three frames and a unique restriction site for the destruction of the uncut or re-ligated template and can be used as part of an off-the-shelf platform for rapidly assembling libraries of antigenic peptides complexed with that MHC allele.

The MHC alleles are also engineered to contain cysteine substitutions at Y84C and A139C to improve folding or increase the binding of the antigenic peptide with the MHC protein. These substitutions allow comPACT2.0 polypeptides and libraries that comprise HLA A33 and HLA B44 alleles which are otherwise not feasible to manufacture.

In addition, the β2M protein can also be mutated (e.g., S88C) to allow it to bind thiol dyes.

Restriction Digest Cloning and Assembly of comPACT2.0 Polynucleotide

Three different methods of inserting the neoantigen via restriction digest are described herein. In the first, the antigenic peptide (NeoE)-encoding primer spans the first restriction site at the 5′ end and the second restriction site at the 3′ end. This primer amplifies off a universal reverse primer encoding the second restriction site, yielding a primer dimer of ˜70 bp.

In the second method, the antigenic peptide-encoding primer spans the second restriction site as the 5′ end and is the reverse complement of the antigen. This primer primes in reverse orientation of the template DNA encoding the signal sequence. Paired with a forward primer spanning the first restriction site sequence, this reaction yields a 70 bp product, or a ˜140 bp product if a forward primer spanning a restriction site farther upstream of the antigen site is used.

In both the first and second methods, the insert is cleaned up on a commercial column, digested with appropriate restriction enzymes, cleaned again on a commercial column, and then ligated with a pre-digested MHC template in a vector. Ligation reactions are transformed into E. coli and plasmids prepared from transformed E. coli are used in mammalian producer cell transfection reactions.

### Example 2: Design and Cloning of comPACT2.0 Polynucleotides Via Primer Annealing

In a third variation on MHC template vector ligation, PCR and restriction digestion were bypassed by annealing two reverse complementary neoantigen-encoding primers. These primers were designed to have 5′ and 3′ ends that begin and terminate in complementary sequences that simulate the overhangs from restriction digestion. The sense and antisense primers were incubated with T4 polynucleotide kinase and ATP to phosphorylate the 5′ ends. When these primers annealed to each other, they formed a double-stranded oligonucleotide sequence that has overhang nucleotides as if it had been digested with a restriction enzyme. The phosphorylated neoantigen insert was ligated into a precut MHC template in a vector. The comPACT2.0 polynucleotide had the same structure as that described in Example 1. The ligation product was then used for PCR amplification of a linear comPACT2.0 amplicon using bookend universal primers to amplify the complete comPACT2.0 gene and sequenced.

Next, E. coli were transformed with the ligation product plasmids and plated onto selective agar plates containing ampicillin. Individual colonies were picked and grown overnight for plasmid purification and sequenced for full gene verification. After sequencing verification, plasmid lots were archived and propagated into larger quantities.

Alternatively, T4 kinase is not used if the precut MHC template vector retains 5′ phosphates on its overhang ends. The annealed antigen insert can then be ligated with the cut MHC vector and the ligation product transformed into E. coli for plasmid production.

### Example 3: Design and Cloning of comPACT2.0 Polynucleotides Via PCR Assembly

Structure of comPACT2.0 Polynucleotides for PCR Assembly:

A fourth method of inserting the neoantigen may also be used. In this method, the neoantigen is inserted into the MHC template which is flanked by an upstream promoter and a downstream polyadenylation signal via polymerase chain reaction to form a 2.5 kb polynucleotide.

In this example, a comPACT2.0 polynucleotide has the following structure: a promoter at the 5′ end; a signal sequence with a first universal target sequence; the antigenic peptide; a second universal target sequence with a linker sequence of predominantly glycine and serine residues (i.e. GlySer linkers); the β2M sequence; a second Gly-Ser linker sequence; an MHC heavy chain allele; a third Gly-Ser linker sequence; a purification cluster; and a polyA sequence. The universal target sequences are not the same in this method.

PCR Assembly of comPACT2.0 Polynucleotides:

In this method, two primers (<60 nt) with a chosen neoantigen sequence are synthesized. The first primer has the neoantigen sequence at the 5′ end followed by a stretch of the second universal target sequence at the 3′ end. The second primer has the reverse complement of the neoantigen sequence at the 5′ end and the reverse complement of the first universal sequence at the 3′ end. These primers are mixed with a DNA fragment encoding the promoter region, signal sequence and first universal target sequence, and another DNA fragment encoding the second universal target sequence, the β2M sequence, MHC allele, purification cluster, and a polyA sequence. Each antigenic peptide primer anneals to its complementary sequence and a PCR reaction is run that amplifies the neoantigen sequence onto either the promoter fragment or the MHC allele fragment. These two newly synthesized fragments now each have the neoantigen sequence. Further PCR reactions, along with primers for the 5′ end of the promoter sequence and 3′ end of the polyA sequence, allow the neoantigen sequences to anneal to each other and prime the assembly of a full-length linear comPACT2.0 amplicon.

The fully assembled linear comPACT2.0 polynucleotide is then cleaned up for direct transfection into mammalian producer cells, bypassing the steps using E. coli and plasmid production altogether.

### Example 4: Disulfide Bond Design for comPACT2.0

Experiments with comPACT1.0 polypeptides and libraries showed that comPACT1.0 polypeptides could not be made for at least HLA A33 and HLA B44 alleles. Without including A33 and B44 alleles in the comPACT2.0 libraries, it is less likely to be able to identify TCRs using comPACT technology for patients and especially for certain ethnicities where A33 and B44 alleles are more prevalent. Accordingly, there was a need to redesign comPACT1.0 to solve this problem. comPACTs are single chain trimer molecules and known algorithms were unable to predict how to stabilize a comPACT to allow for the expression of and manufacture of a comPACT with an HLA A33 or HLA B44 allele. Accordingly, different cysteine mutations were explored to find the correct mutations to allow for stabilization of the HLA and manufacture and expression of the comPACT.

It was determined that engineering a Y84C and an A139C mutation (i.e., the comPACT2.0) in the HLAs allowed for a general increase in comPACT stability, expression, and manufacture and even saved previously unusable HLAs such as HLA A33 and B44 (FIG. 1). Furthermore, the stabilization resulting from the disulfide bond also proved to increase the dynamics of the allele binding.

Additional experiments were performed to show that the Y84C and an A139C mutation (i.e., the comPACT2.0) did not reduce the function of any other HLAs that previously worked using the comPACT1.0 technology.

This experiment is the first time the Y84C and A139C mutations were used successfully in a single chain trimer format to benefit the proper folding of an HLA.

### Example 5: comPACT2.0 is Superior to comPACT1.0

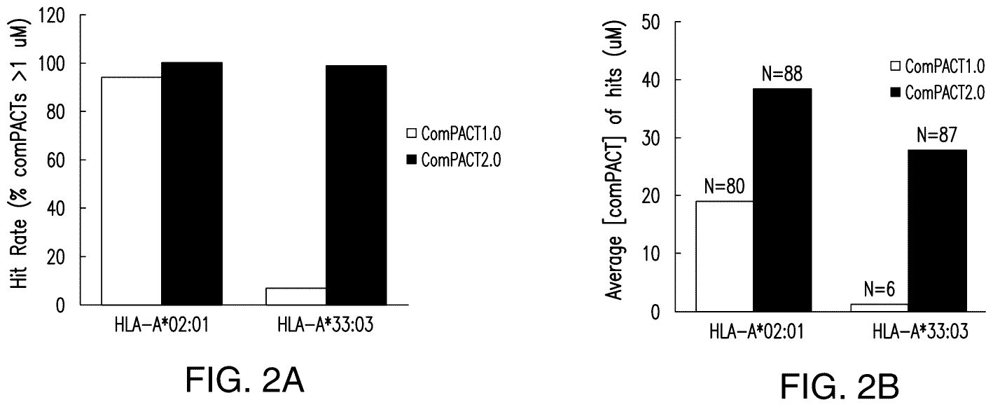

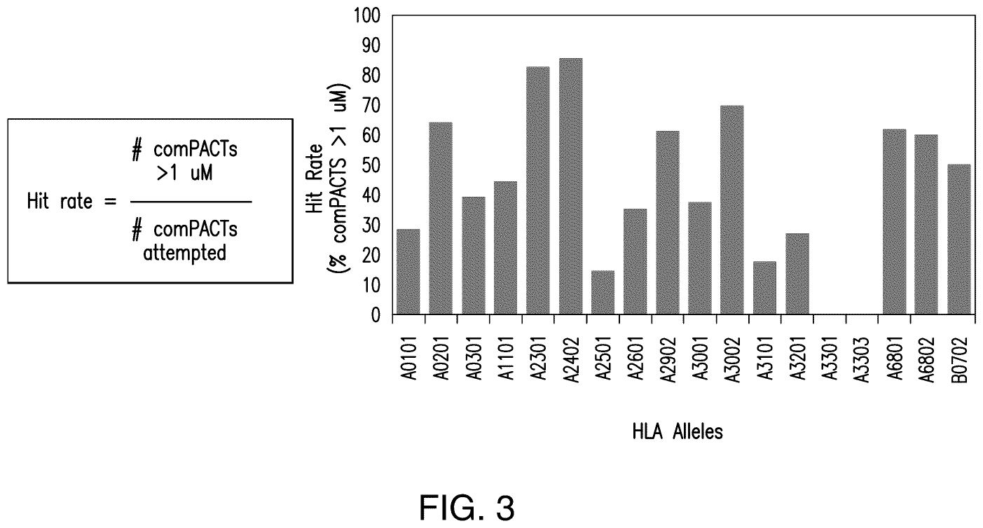

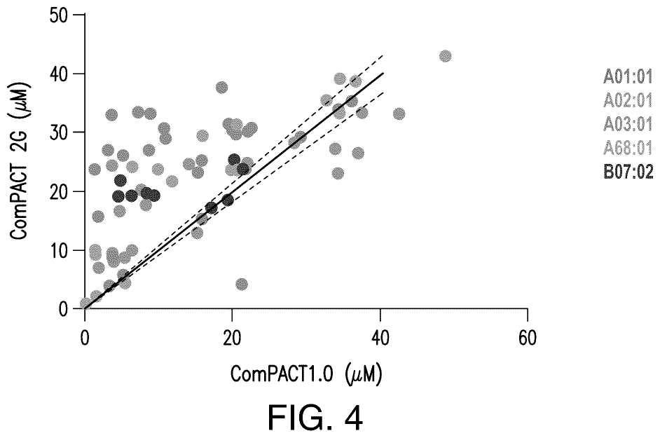

Experiments were performed to test whether comPACT2.0 polypeptides had a beneficial or negative effect on HLAs that were successful in the comPACT1.0 architecture. As shown in FIG. 2A, comPACT2.0 polypeptides were successful for HLA A02:01 (an HLA that was very successful with the comPACT1.0 technology). There was no reduced expression or manufacturing decline using the comPACT2.0 architecture.

Also shown in FIG. 2A is that while there was no significant increase or decrease with HLA A02:01, there was a significant increase with A33:03 (an HLA that could not be expressed or manufactured using the comPACT1.0 technology). Specifically, comPACT1.0 only gave a 6% hit rate for A33:03; however, comPACT2.0 gave a 99% hit rate. This shows that the comPACT2.0 technology with the Y84C and A139C mutations was able to save the A33 allele and allow it to become part of a comPACT2.0 library. A similar trend significant improvement for the A33 allele is shown by the average hit (i.e., expression) rates in FIG. 2B showing that while A33:03 could not be expressed and made using comPACT1.0 technology, it could efficiently be expressed and made using the comPACT2.0 technology.

FIG. 2B provided surprising results. There was no expectation that the average number of hits (i.e., expression rate) would improve for A02:01 by using the comPACT2.0 technology compared to the prior comPACT1.0 technology. Surprisingly, the average number of hits approximately doubled for A02:01.

Furthermore, by means of example, comPACT2.0 improved the yield for many neoepitopes regardless of their HLA alleles (i.e., even yields that were previously thought to be good using the comPACT1.0 technology were improved using the comPACT2.0 technology).

### Example 6: comPACT2.0 Linker Selection

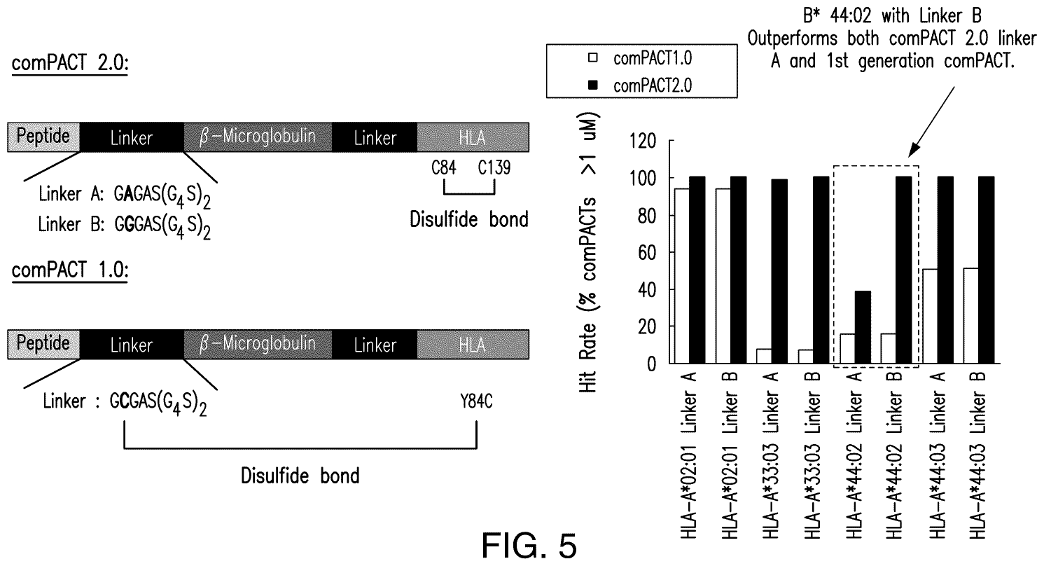

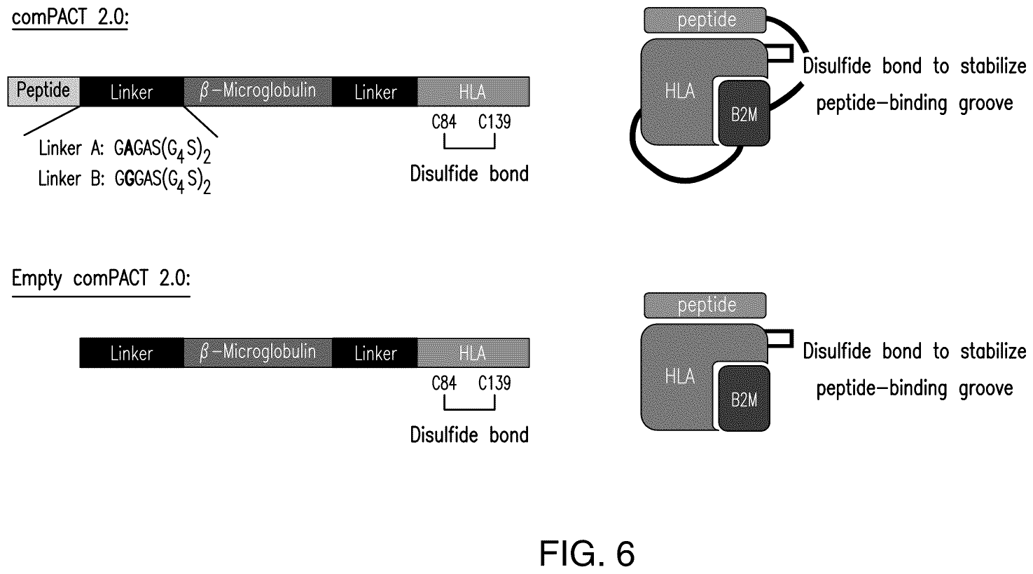

Experiments were performed to determine which linkers provided the optimum dynamic flexibility for the comPACT2.0 polypeptide structure. As shown in FIG. 5, experiments were performed on multiple GS linkers, data of two linkers shown, and data on other linkers not shown. Because the GS linker of comPACT1.0 polypeptides was designed to have a cysteine to form a disulfide bond between the linker and the HLA, the GS linker for comPACT2.0 had to be redesigned to remove the engineered cysteine to avoid misfolding and disulfide bonds between the HLA and linker. Accordingly, Linker A (GAGAS(G₄S)₂) and Linker B (GGGAS(G₄S)₂) were tested to see if either would work and if had an unpredicted benefit (FIG. 5).

As shown in FIG. 5, Linker B consistently outperformed Linker B when it came to comPACT2.0 polypeptide expression.

### Example 7: Empty comPACT2.0

Experiments were performed to determine whether it would be possible to obtain an empty comPACT2.0 peptide into which antigenic peptide can be loaded. As illustrated in FIG. 6, empty comPACT2.0 peptides are not covalently bound to the antigenic peptide but interact with it to form a stabilized multimeric protein. In order to load a free soluble antigenic peptide (e.g., 8-mer to 11-mer) into a specific HLA allele, 100 μM of a soluble antigenic peptide is incubated with 10 μM of empty comPACT2.0 for 15 minutes at 4C, after which a peptide loaded comPACT2.0 is obtained.

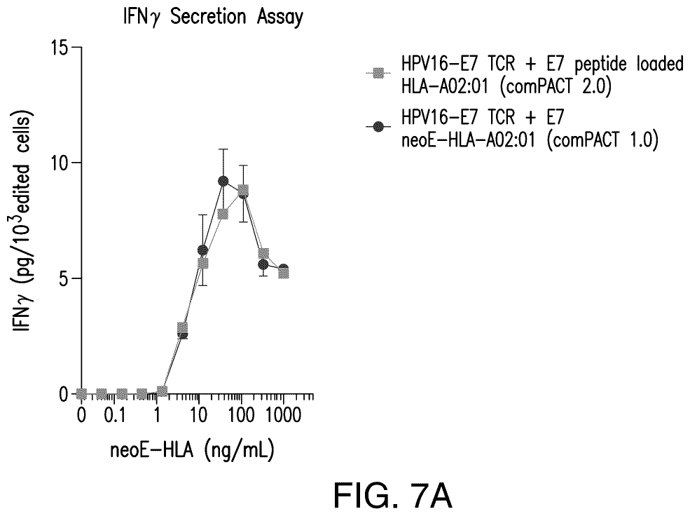

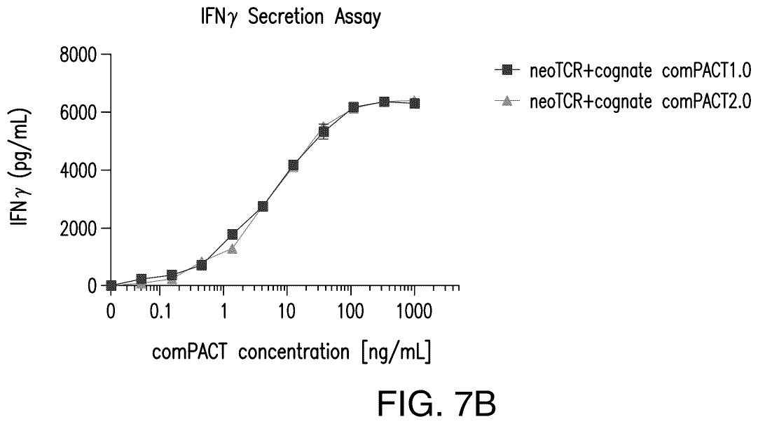

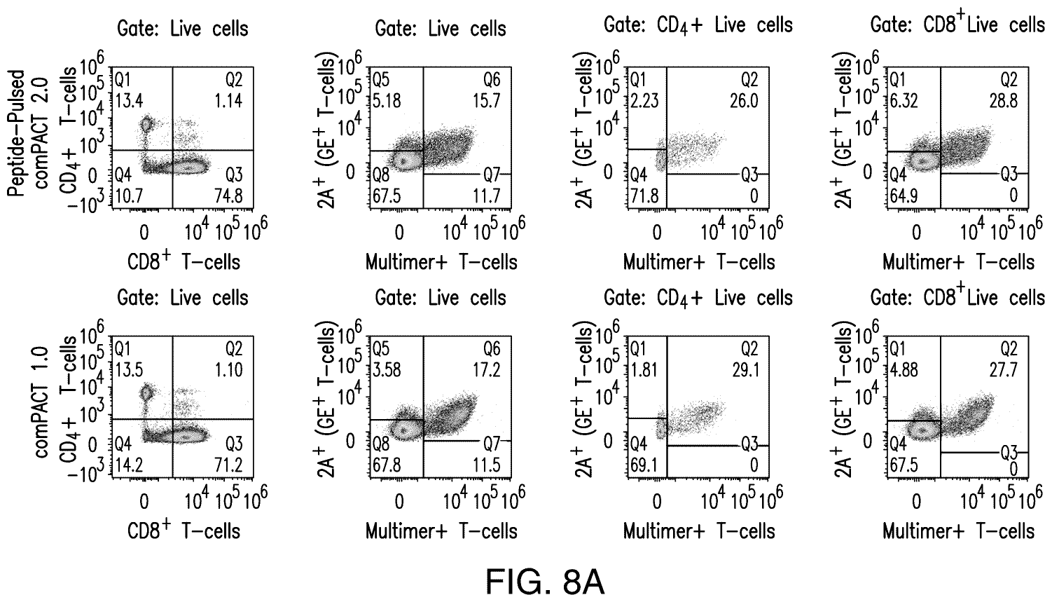

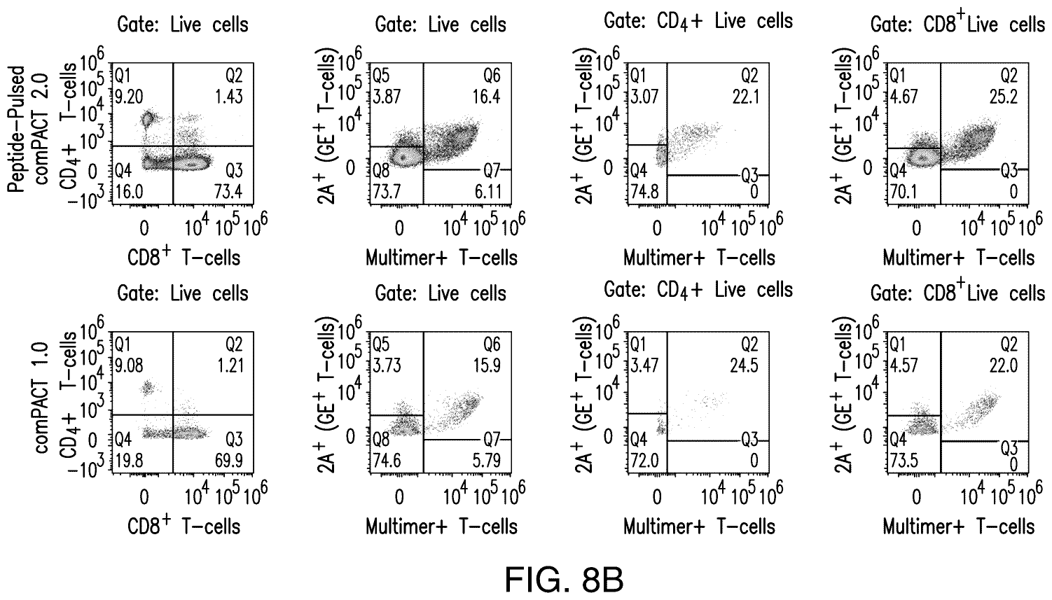

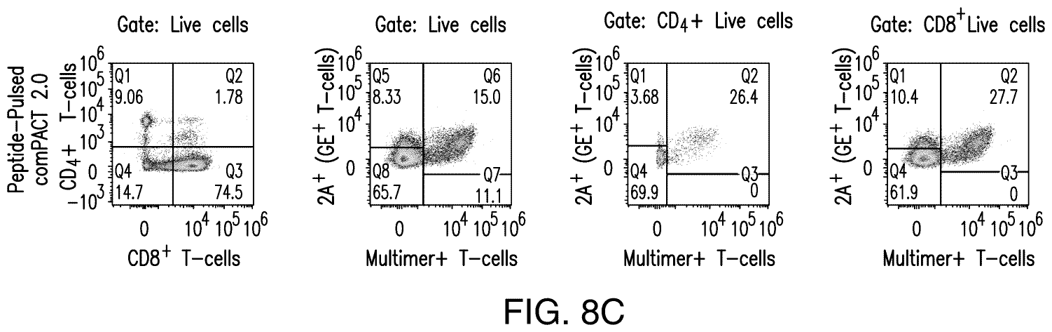

Peptide loaded comPACT2.0 and comPACT1.0, each including a HPV16-E7 derived neoantigen, were compared in functional T cell assays. As shown in FIGS. 7A and 7B, NeoTCR edited healthy donor T cells, generated using the non-viral gene editing process, secreted comparable IFNγ upon activation with increasing concentrations cognate of neoE-HLA-A02:01. FACS analysis using HPV16-E7 derived neoantigens and Neo12 neoantigens showed similar effect (FIGS. 8A and 8B).

Next, it was observed that comPACT1.0 and comPACT2.0 peptides failed to bind HPV16-E6 derived neoantigens (data not shown). The inventors of the present disclosure hypothesized that the HPV16-E6 derived neoantigen contains the amino acid cysteine and that, when expressed as a linked component of comPACT1.0 or comPACT2.0, results in secondary adduct formation (e.g., cysteinylation) to the cysteine-containing peptide sequence. This adduct formation can partially or completely disrupt the ability of the TCR to recognize and bind to the cognate comPACT1.0 or comPACT2.0. Thus, the use of peptide loaded comPACT2.0 should overcome this problem. Indeed, as shown in FIG. 8C, NeoTCR edited healthy donor T cells, generated using non-viral gene editing process, demonstrated multimer binding with cognate neoE-HLA-A02:01 generated as peptide loaded comPACT 2.0 (e.g., peptide-pulsed comPACT 2.0).

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A polynucleotide comprising, in a 5′ to 3′ orientation, a sequence insert, a beta 2 Microglobulin (β2M) sequence, and a Major Histocompatibility Complex (MHC) allele sequence, wherein the sequence insert comprises a stop codon in each open reading frame, and wherein the MHC allele sequence comprises a Y84C mutation and a A139C mutation.

2. The polynucleotide of claim 1, wherein the sequence insert is flanked by a first universal target sequence and/or a second universal target sequence.

3. A polynucleotide comprising, in a 5′ to 3′ orientation, a first universal target sequence, a nucleotide sequence encoding an antigenic peptide, a second universal target sequence, a Beta 2 Microglobulin (β2M) sequence, and a Major Histocompatibility Complex (MHC) allele sequence, wherein the MHC allele sequence comprises a Y84C mutation and a A139C mutation.

4. The polynucleotide of claim 1 further comprising a linker positioned 5′ end of the β2M sequence.

5. A polynucleotide comprising, in a 5′ to 3′ orientation, a sequence encoding a linker, a Beta 2 Microglobulin (β2M) sequence, and a Major Histocompatibility Complex (MHC) allele sequence, wherein the MHC allele sequence comprises a Y84C mutation and a A139C mutation.

6. The polynucleotide of claim 5, wherein the antigenic peptide is an HPV neoantigen.

7. The polynucleotide of claim 1, wherein the β2M allele is a human β2M allele and/or the MHC allele is a human MHC allele.

8. The polynucleotide of claim 7, wherein the human MHC allele is an HLA allele selected from the group consisting of: HLA-A*01:01, HLA-A*02:01, HLA-A*03:01, HLA-A*24:02, HLA-A*30:02, HLA-A*31:01, HLA-A*32:01, HLA-A*33:01, HLA-A*68:01, HLA-A*11:01, HLA-A*23:01, HLA-A*30:01, HLA-A*33:03, HLA-A*25:01, HLA-A*26:01, HLA-A*29:02, HLA-A*68:02, HLA-B*07:02, HLA-B*14:02, HLA-B*18:01, HLA-B*27:02, HLA-B*39:01, HLA-B*40:01, HLA-B*44:02, HLA-B*46:01, HLA-B*50:01, HLA-B*57:01, HLA-B*58:01, HLA-B*08:01, HLA-B*15:01, HLA-B*15:03, HLA-B*35:01, HLA-B*40:02, HLA-B*42:01, HLA-B*44:03, HLA-B*51:01, HLA-B*53:01, HLA-B*13:02, HLA-B*15:07, HLA-B*27:05, HLA-B*35:03, HLA-B*37:01, HLA-B*38:01, HLA-B*41:02, HLA-B*44:05, HLA-B*49:01, HLA-B*52:01, HLA-B*55:01, HLA-C*02:02, HLA-C*03:04, HLA-C*05:01, HLA-C*07:01, HLA-C*01:02, HLA-C*04:01, HLA-C*06:02, HLA-C*07:02, HLA-C*16:01, HLA-C*03:03, HLA-C*07:04, HLA-C*08:01, HLA-C*08:02, HLA-C*12:02, HLA-C*12:03, HLA-C*14:02, HLA-C*15:02, and HLA-C*17:01.

9. The polynucleotide of claim 8, wherein the HLA allele comprises the amino acid sequence selected from the group consisting of SEQ ID NOs: 1-66 or 137-202.

10. The polynucleotide of claim 8, wherein the human MHC allele is selected from the group consisting of HLA-A*33:01, HLA-A*33:03, HLA-B*44:02, HLA-B*44:03, and HLA-B*44:025.

11. The polynucleotide of claim 2, wherein the first and second universal target sequences comprise a polymerase chain reaction (PCR) primer target site and/or a restriction enzyme cleavage site.

12. The polynucleotide of claim 1 further comprising a sequence encoding a signal sequence and/or a purification cluster sequence positioned at the 3′ end of the polynucleotide, wherein the purification cluster comprises a first affinity tag sequence, a protease cleavage site sequence, and a second affinity tag sequence.

13. An expression construct comprising the polynucleotide of claim 1.

14. A vector comprising the polynucleotide of claim 1.

15. A polypeptide encoded by the polynucleotide of claim 1.

16. A protein complex comprising a first polypeptide comprising an antigenic peptide and a second polypeptide encoded by the polynucleotide of claim 6.

17. A host cell comprising the polynucleotide of claim 1.

18. A library comprising at least two polynucleotides of claim 1, wherein at least one polynucleotide comprises a human MHC allele selected from the group consisting of HLA-A*33:01, HLA-A*33:03, HLA-B*44:02, HLA-B*44:03, and HLA-B*44:025.

19. A kit comprising a library of claim 17 and instructions for use.

20. A method for isolating an antigen-specific T cell, the method comprising:
a) providing a plurality of particles, wherein each particle comprises a polypeptide of claim 15;
b) obtaining a sample known or suspected to comprise one or more T cells;
c) contacting the plurality of particles with the sample, wherein the contacting comprises providing conditions sufficient for a single T cell to bind the polypeptide attached to the particle, and
d) isolating the single T cell associated with the particle.


SEQUENCE LISTING (202 sequences)

SEQ ID NO: 1 - protein, 276 aa, synthetic construct
  REGION 1..276: note = Synthetic: HLA-A*01:01
GSHSMRYFFTSVSRPGRGEPRFIAVGYVDDTQFVRFDSDAASQKMEPRAPWIEQEGPEYWDQETRNMKAHSQTDRANLGTLRGCYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 2 - protein, 276 aa, synthetic construct
  REGION 1..276: note = Synthetic: HLA-A*02:01
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEP

SEQ ID NO: 3 - protein, 276 aa, synthetic construct
  REGION 1..276: note = Synthetic: HLA-A*03:01
GSHSMRYFFTSVSRPGRGEPRFIAVGYVDDTQFVRFDSDAASQRMEPRAPWIEQEGPEYWDQETRNVKAQSQTDRVDLGTLRGCYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 4 - protein, 276 aa, synthetic construct
  REGION 1..276: note = Synthetic: HLA-A*24:02
GSHSMRYFSTSVSRPGRGEPRFIAVGYVDDTQFVRFDSDAASQRMEPRAPWIEQEGPEYWDEETGKVKAHSQTDRENLRIALRCYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 5 - protein, 276 aa, synthetic construct
  REGION 1..276: note = Synthetic: HLA-A*30:02
GSHSMRYFSTSVSRPGSGEPRFIAVGYVDDTQFVRFDSDAASQRMEPRAPWIEQERPEYWDQETRNVKAHSQTDRENLGTLRGCYNQSEAGSHTIQIMYGCDVGSDGRFLRGYEQHAYDGKDYIALNEDLRSWTAADMAAQITQRKWEAARRAEQLRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 6 - protein, 276 aa, synthetic construct
  REGION 1..276: note = Synthetic: HLA-A*31:01
GSHSMRYFTTSVSRPGRGEPRFIAVGYVDDTQFVRFDSDAASQRMEPRAPWIEQERPEYWDQETRNVKAHSQIDRVDLGTLRGCYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 7 - protein, 276 aa, synthetic construct
  REGION 1..276: note = Synthetic: HLA-A*32:01
GSHSMRYFFTSVSRPGRGEPRFIAVGYVDDTQFVRFDSDAASQRMEPRAPWIEQEGPEYWDQETRNVKAHSQTDRESLRIALRCYNQSEAGSHTIQMMYGCDVGPDGRLLRGYQQDAYDGKDYIALNEDLRSWTAADMAAQITQRKWEAARVAEQLRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 8 - protein, 276 aa, synthetic construct
  REGION 1..276: note = Synthetic: HLA-A*33:01
GSHSMRYFTTSVSRPGRGEPRFIAVGYVDDTQFVRFDSDAASQRMEPRAPWIEQEGPEYWDRNTRNVKAHSQIDRVDLGTLRGCYNQSEAGSHTIQMMYGCDVGSDGRFLRGYQQDAYDGKDYIALNEDLRSWTAADMAAQITQRKWEAARVAEQLRAYLEGTCVEWLRRHLENGKETLQRTDPPRTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 9 - protein, 276 aa, synthetic construct
  REGION 1..276: note = Synthetic: HLA-A*68:01
GSHSMRYFYTSVSRPGRGEPRFIAVGYVDDTQFVRFDSDAASQRMEPRAPWIEQEGPEYWDRNTRNVKAQSQTDRVDLGTLRGCYNQSEAGSHTIQMMYGCDVGSDGRFLRGYRQDAYDGKDYIALKEDLRSWTAADMAAQTTKHKWEAAHVAEQWRAYLEGTCVEWLRRYLENGKETLQRTDAPKTHMTHHAVSDHEATLRCWALSFYPAEITLTWQRDGEDQTQDTELVETRPAGDGTFQKWVAVVVPSGQEQRYTCHVQHEGLPKPLTLRWEP

SEQ ID NO: 10 - protein, 276 aa, synthetic construct
  REGION 1..276: note = Synthetic: HLA-B*07:02
GSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 11 - protein, 276 aa, synthetic construct
  REGION 1..276: note = Synthetic: HLA-B*14:02
GSHSMRYFYTAVSRPGRGEPRFISVGYVDDTQFVRFDSDAASPREEPRAPWIEQEGPEYWDRNTQICKTNTQTDRESLRNLRGCYNQSEAGSHTLQWMYGCDVGPDGRLLRGYNQFAYDGKDYIALNEDLSSWTAADTAAQITQRKWEAAREAEQLRAYLEGTCVEWLRRHLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 12 - protein, 276 aa, synthetic construct
  REGION 1..276: note = Synthetic: HLA-B*18:01
GSHSMRYFHTSVSRPGRGEPRFISVGYVDGTQFVRFDSDAASPRTEPRAPWIEQEGPEYWDRNTQISKTNTQTYRESLRNLRGCYNQSEAGSHTLQRMYGCDVGPDGRLLRGHDQSAYDGKDYIALNEDLSSWTAADTAAQITQRKWEAARVAEQLRAYLEGTCVEWLRRHLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 13 - protein, 276 aa, synthetic construct
  REGION 1..276: note = Synthetic: HLA-B*27:02
GSHSMRYFHTSVSRPGRGEPRFITVGYVDDTLFVRFDSDAASPREEPRAPWIEQEGPEYWDRETQICKAKAQTDRENLRIALRCYNQSEAGSHTLQNMYGCDVGPDGRLLRGYHQDAYDGKDYIALNEDLSSWTAADTAAQITQRKWEAARVAEQLRAYLEGECVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 14 - protein, 276 aa, synthetic construct
  REGION 1..276: note = Synthetic: HLA-B*39:01
GSHSMRYFYTSVSRPGRGEPRFISVGYVDDTQFVRFDSDAASPREEPRAPWIEQEGPEYWDRNTQICKTNTQTDRESLRNLRGCYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 15 - protein, 276 aa, synthetic construct
  REGION 1..276: note = Synthetic: HLA-B*40:01
GSHSMRYFHTAMSRPGRGEPRFITVGYVDDTLFVRFDSDATSPRKEPRAPWIEQEGPEYWDRETQISKTNTQTYRESLRNLRGCYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 16 - protein, 276 aa, synthetic construct
  REGION 1..276: note = Synthetic: HLA-B*44:02
GSHSMRYFYTAMSRPGRGEPRFITVGYVDDTLFVRFDSDATSPRKEPRAPWIEQEGPEYWDRETQISKTNTQTYRENLRTALRCYNQSEAGSHIIQRMYGCDVGPDGRLLRGYDQDAYDGKDYIALNEDLSSWTAADTAAQITQRKWEAARVAEQDRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEP

SEQ ID NO: 17 - protein, 276 aa, synthetic construct
  REGION 1..276: note = Synthetic: HLA-B*46:01
GSHSMRYFYTAMSRPGRGEPRFIAVGYVDDTQFVRFDSDAASPRMAPRAPWIEQEGPEYWDRETQKYKRQAQTDRVSLRNLRGCYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 18 - protein, 276 aa, synthetic construct
  REGION 1..276: note = Synthetic: HLA-B*50:01
GSHSMRYFHTAMSRPGRGEPRFITVGYVDDTLFVRFDSDATSPRKEPRAPWIEQEGPEYWDRETQISKTNTQTYRESLRNLRGCYNQSEAGSHTWQRMYGCDLGPDGRLLRGYNQLAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 19 - protein, 276 aa, synthetic construct
  REGION 1..276: note = Synthetic: HLA-B*57:01
GSHSMRYFYTAMSRPGRGEPRFIAVGYVDDTQFVRFDSDAASPRMAPRAPWIEQEGPEYWDGETRNMKASAQTYRENLRIALRCYNQSEAGSHIIQVMYGCDVGPDGRLLRGHDQSAYDGKDYIALNEDLSSWTAADTAAQITQRKWEAARVAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 20 - protein, 276 aa, synthetic construct
  REGION 1..276: note = Synthetic: HLA-B*58:01
GSHSMRYFYTAMSRPGRGEPRFIAVGYVDDTQFVRFDSDAASPRTEPRAPWIEQEGPEYWDGETRNMKASAQTYRENLRIALRCYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 21 - protein, 276 aa, synthetic construct
  REGION 1..276: note = Synthetic: HLA-C*02:02
CSHSMRYFYTAVSRPSRGEPHFIAVGYVDDTQFVRFDSDAASPRGEPRAPWVEQEGPEYWDRETQKYKRQAQTDRVNLRKLRGCYNQSEAGSHTLQRMYGCDLGPDGRLLRGYDQSAYDGKDYIALNEDLRSWTAADTAAQITQRKWEAAREAEQWRAYLEGECVEWLRRYLENGKETLQRAEHPKTHVTHHPVSDHEATLRCWALGFYPTEITLTWQRDGEDQTQDTELVETRPAGDGTFQKWAAVVVPSGEEQRYTCHVQHEGLPEPLTLRWEP

SEQ ID NO: 22 - protein, 276 aa, synthetic construct
  REGION 1..276: note = Synthetic: HLA-C*03:04
GSHSMRYFYTAVSRPGRGEPHFIAVGYVDDTQFVRFDSDAASPRGEPRAPWVEQEGPEYWDRETQKYKRQAQTDRVSLRNLRGCYNQSEAGSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 23 - protein, 276 aa, synthetic construct
  REGION 1..276: note = Synthetic: HLA-C*05:01
CSHSMRYFYTAVSRPGRGEPRFIAVGYVDDTQFVQFDSDAASPRGEPRAPWVEQEGPEYWDRETQKYKRQAQTDRVNLRKLRGCYNQSEAGSHTLQRMYGCDLGPDGRLLRGYNQFAYDGKDYIALNEDLRSWTAADKAAQITQRKWEAAREAEQRRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGP

SEQ ID NO: 24 - protein, 276 aa, synthetic construct
  REGION 1..276: note = Synthetic: HLA-C*07:01
CSHSMRYFDTAVSRPGRGEPRFISVGYVDDTQFVRFDSDAASPRGEPRAPWVEQEGPEYWDRETQNYKRQAQADRVSLRNLRGCYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 25 - protein, 276 aa, synthetic construct
  REGION 1..276: note = Synthetic: HLA-A*11:01
GSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 26 - protein, 276 aa, synthetic construct
  REGION 1..276: note = Synthetic: HLA-A*23:01
GSHSMRYFSTSVSRPGRGEPRFIAVGYVDDTQFVRFDSDAASQRMEPRAPWIEQEGPEYWDEETGKVKAHSQTDRENLRIALRCYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 27 - protein, 276 aa, synthetic construct
  REGION 1..276: note = Synthetic: HLA-A*30:01
GSHSMRYFSTSVSRPGSGEPRFIAVGYVDDTQFVRFDSDAASQRMEPRAPWIEQERPEYWDQETRNVKAQSQTDRVDLGTLRGCYNQSEAGSHTIQIMYGCDVGSDGRFLRGYEQHAYDGKDYIALNEDLRSWTAADMAAQITQRKWEAARWAEQLRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 28 - protein, 276 aa, synthetic construct
  REGION 1..276: note = Synthetic: HLA-A*33:03
GSHSMRYFTTSVSRPGRGEPRFIAVGYVDDTQFVRFDSDAASQRMEPRAPWIEQEGPEYWDRNTRNVKAHSQIDRVDLGTLRGCYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 29 - protein, 276 aa, synthetic construct
  REGION 1..276: note = Synthetic: HLA-B*08:01
GSHSMRYFDTAMSRPGRGEPRFISVGYVDDTQFVRFDSDAASPREEPRAPWIEQEGPEYWDRNTQIFKTNTQTDRESLRNLRGCYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 30 - protein, 276 aa, synthetic construct
  REGION 1..276: note = Synthetic: HLA-B*15:01
GSHSMRYFYTAMSRPGRGEPRFIAVGYVDDTQFVRFDSDAASPRMAPRAPWIEQEGPEYWDRETQISKTNTQTYRESLRNLRGCYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 31 - protein, 276 aa, synthetic construct
  REGION 1..276: note = Synthetic: HLA-B*15:03
GSHSMRYFYTAMSRPGRGEPRFISVGYVDDTQFVRFDSDAASPREEPRAPWIEQEGPEYWDRETQISKTNTQTYRESLRNLRGCYNQSEAGSHTLQRMYGCDVGPDGRLLRGHDQSAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 32 - protein, 276 aa, synthetic construct
  REGION 1..276: note = Synthetic: HLA-B*35:01
GSHSMRYFYTAMSRPGRGEPRFIAVGYVDDTQFVRFDSDAASPRTEPRAPWIEQEGPEYWDRNTQIFKTNTQTYRESLRNLRGCYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 33 - protein, 276 aa, synthetic construct
  REGION 1..276: note = Synthetic: HLA-B*40:02
GSHSMRYFHTSVSRPGRGEPRFITVGYVDDTLFVRFDSDATSPRKEPRAPWIEQEGPEYWDRETQISKTNTQTYRESLRNLRGCYNQSEAGSHTLQSMYGCDVGPDGRLLRGHNQYAYDGKDYIALNEDLRSWTAADTAAQITQRKWEAARVAEQLRAYLEGECVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 34 - protein, 276 aa, synthetic construct
  REGION 1..276: note = Synthetic: HLA-B*42:01
GSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 35 - protein, 276 aa, synthetic construct
  REGION 1..276: note = Synthetic: HLA-B*44:03
GSHSMRYFYTAMSRPGRGEPRFITVGYVDDTLFVRFDSDATSPRKEPRAPWIEQEGPEYWDRETQISKTNTQTYRENLRTALRCYNQSEAGSHIIQRMYGCDVGPDGRLLRGYDQDAYDGKDYIALNEDLSSWTAADTAAQITQRKWEAARVAEQLRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEP

SEQ ID NO: 36 - protein, 276 aa, synthetic construct
  REGION 1..276: note = Synthetic: HLA-B*51:01
GSHSMRYFYTAMSRPGRGEPRFIAVGYVDDTQFVRFDSDAASPRTEPRAPWIEQEGPEYWDRNTQIFKTNTQTYRENLRIALRCYNQSEAGSHTWQTMYGCDVGPDGRLLRGHNQYAYDGKDYIALNEDLSSWTAADTAAQITQRKWEAAREAEQLRAYLEGLCVEWLRRHLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 37 - protein, 276 aa, synthetic construct
  REGION 1..276: note = Synthetic: HLA-B*53:01
GSHSMRYFYTAMSRPGRGEPRFIAVGYVDDTQFVRFDSDAASPRTEPRAPWIEQEGPEYWDRNTQIFKTNTQTYRENLRIALRCYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 38 - protein, 276 aa, synthetic construct
  REGION 1..276: note = Synthetic: HLA-C*01:02
CSHSMKYFFTSVSRPGRGEPRFISVGYVDDTQFVRFDSDAASPRGEPRAPWVEQEGPEYWDRETQKYKRQAQTDRVSLRNLRGCYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEP

SEQ ID NO: 39 - protein, 276 aa, synthetic construct
  REGION 1..276: note = Synthetic: HLA-C*04:01
GSHSMRYFSTSVSWPGRGEPRFIAVGYVDDTQFVRFDSDAASPRGEPREPWVEQEGPEYWDRETQKYKRQAQADRVNLRKLRGCYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWKP

SEQ ID NO: 40 - protein, 275 aa, synthetic construct
  REGION 1..275: note = Synthetic: HLA-C*06:02
CSHSMRYFDTAVSRPGRGEPRFISVGYVDDTQFVRFDSDAASPRGEPRAPWVEQEGPEYWDRETQKYKRQAQADRVNLRKLRGCYNQSEDGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWE

SEQ ID NO: 41 - protein, 276 aa, synthetic construct
  REGION 1..276: note = Synthetic: HLA-C*07:02
CSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 42 - protein, 276 aa, synthetic construct
  REGION 1..276: note = Synthetic: HLA-C*16:01
CSHSMRYFYTAVSRPGRGEPRFIAVGYVDDTQFVRFDSDAASPRGEPRAPWVEQEGPEYWDRETQKYKRQAQTDRVSLRNLRGCYNQSEAGSHTLQWMYGCDLGPDGRLLRGYDQSAYDGKDYIALNEDLRSWTAADTAAQITQRKWEAARAAEQQRAYLEGTCVEWLRRYLENGKETLQRAEHPKTHVTHHLVSDHEATLRCWALGFYPAEITLTWQRDGEDQTQDTELVETRPAGDGTFQKWAAVVVPSGEEQRYTCHVQHEGLPEPLTLRWEP

SEQ ID NO: 43 - protein, 276 aa, synthetic construct
  REGION 1..276: note = Synthetic: HLA-A*25:01
GSHSMRYFYTSVSRPGRGEPRFIAVGYVDDTQFVRFDSDAASQRMEPRAPWIEQEGPEYWDRNTRNVKAHSQTDRESLRIALRCYNQSEDGSHTIQRMYGCDVGPDGRFLRGYQQDAYDGKDYIALNEDLRSWTAADMAAQITQRKWETAHEAEQWRAYLEGRCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 44 - protein, 276 aa, synthetic construct
  REGION 1..276: note = Synthetic: HLA-A*26:01
GSHSMRYFYTSVSRPGRGEPRFIAVGYVDDTQFVRFDSDAASQRMEPRAPWIEQEGPEYWDRNTRNVKAHSQTDRANLGTLRGCYNQSEDGSHTIQRMYGCDVGPDGRFLRGYQQDAYDGKDYIALNEDLRSWTAADMAAQITQRKWETAHEAEQWRAYLEGRCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 45 - protein, 276 aa, synthetic construct
  REGION 1..276: note = Synthetic: HLA-A*29:02
GSHSMRYFTTSVSRPGRGEPRFIAVGYVDDTQFVRFDSDAASQRMEPRAPWIEQEGPEYWDLQTRNVKAQSQTDRANLGTLRGCYNQSEAGSHTIQMMYGCDVGSDGRFLRGYRQDAYDGKDYIALNEDLRSWTAADMAAQITQRKWEAARVAEQLRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 46 - protein, 276 aa, synthetic construct
  REGION 1..276: note = Synthetic: HLA-A*68:02
GSHSMRYFYTSMSRPGRGEPRFIAVGYVDDTQFVRFDSDAASQRMEPRAPWIEQEGPEYWDRNTRNVKAQSQTDRVDLGTLRGCYNQSEAGSHTIQRMYGCDVGPDGRFLRGYHQYAYDGKDYIALKEDLRSWTAADMAAQTTKHKWEAAHVAEQWRAYLEGTCVEWLRRYLENGKETLQRTDAPKTHMTHHAVSDHEATLRCWALSFYPAEITLTWQRDGEDQTQDTELVETRPAGDGTFQKWVAVVVPSGQEQRYTCHVQHEGLPKPLTLRWEP

SEQ ID NO: 47 - protein, 276 aa, synthetic construct
  REGION 1..276: note = Synthetic: HLA-B*13:02
GSHSMRYFYTAMSRPGRGEPRFITVGYVDDTQFVRFDSDATSPRMAPRAPWIEQEGPEYWDRETQISKTNTQTYRENLRTALRCYNQSEAGSHTWQTMYGCDLGPDGRLLRGHNQLAYDGKDYIALNEDLSSWTAADTAAQITQLKWEAARVAEQLRAYLEGECVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 48 - protein, 276 aa, synthetic construct
  REGION 1..276: note = Synthetic: HLA-B*15:07
GSHSMRYFYTAMSRPGRGEPRFIAVGYVDDTQFVRFDSDAASPRMAPRAPWIEQEGPEYWDRETQISKTNTQTYRESLRNLRGCYNQSEAGSHTLQSMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 49 - protein, 276 aa, synthetic construct
  REGION 1..276: note = Synthetic: HLA-B*27:05
GSHSMRYFHTSVSRPGRGEPRFITVGYVDDTLFVRFDSDAASPREEPRAPWIEQEGPEYWDRETQICKAKAQTDREDLRTLLRCYNQSEAGSHTLQNMYGCDVGPDGRLLRGYHQDAYDGKDYIALNEDLSSWTAADTAAQITQRKWEAARVAEQLRAYLEGECVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 50 - protein, 276 aa, synthetic construct
  REGION 1..276: note = Synthetic: HLA-B*35:03
GSHSMRYFYTAMSRPGRGEPRFIAVGYVDDTQFVRFDSDAASPRTEPRAPWIEQEGPEYWDRNTQIFKTNTQTYRESLRNLRGCYNQSEAGSHIIQRMYGCDLGPDGRLLRGHDQFAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 51 - protein, 276 aa, synthetic construct
  REGION 1..276: note = Synthetic: HLA-B*37:01
GSHSMRYFHTSVSRPGRGEPRFISVGYVDDTQFVRFDSDAASPRTEPRAPWIEQEGPEYWDRETQISKTNTQTYREDLRTLLRCYNQSEAGSHTIQRMSGCDVGPDGRLLRGYNQFAYDGKDYIALNEDLSSWTAADTAAQITQRKWEAARVAEQDRAYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 52 - protein, 276 aa, synthetic construct
  REGION 1..276: note = Synthetic: HLA-B*38:01
GSHSMRYFYTSVSRPGRGEPRFISVGYVDDTQFVRFDSDAASPREEPRAPWIEQEGPEYWDRNTQICKTNTQTYRENLRIALRCYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 53 - protein, 276 aa, synthetic construct
  REGION 1..276: note = Synthetic: HLA-B*41:02
GSHSMRYFHTAMSRPGRGEPRFITVGYVDDTLFVRFDSDATSPRKEPRAPWIEQEGPEYWDRETQISKTNTQTYRESLRNLRGCYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 54 - protein, 276 aa, synthetic construct
  REGION 1..276: note = Synthetic: HLA-B*44:05
GSHSMRYFYTAMSRPGRGEPRFITVGYVDDTLFVRFDSDATSPRKEPRAPWIEQEGPEYWDRETQISKTNTQTYRENLRTALRCYNQSEAGSHIIQRMYGCDVGPDGRLLRGYDQYAYDGKDYIALNEDLSSWTAADTAAQITQRKWEAARVAEQDRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEP

SEQ ID NO: 55 - protein, 276 aa, synthetic construct
  REGION 1..276: note = Synthetic: HLA-B*49:01
GSHSMRYFHTAMSRPGRGEPRFITVGYVDDTLFVRFDSDATSPRKEPRAPWIEQEGPEYWDRETQISKTNTQTYRENLRIALRCYNQSEAGSHTWQRMYGCDLGPDGRLLRGYNQLAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 56 - protein, 276 aa, synthetic construct
  REGION 1..276: note = Synthetic: HLA-B*52:01
GSHSMRYFYTAMSRPGRGEPRFIAVGYVDDTQFVRFDSDAASPRTEPRAPWIEQEGPEYWDRETQISKTNTQTYRENLRIALRCYNQSEAGSHTWQTMYGCDVGPDGRLLRGHNQYAYDGKDYIALNEDLSSWTAADTAAQITQRKWEAAREAEQLRAYLEGLCVEWLRRHLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 57 - protein, 276 aa, synthetic construct
  REGION 1..276: note = Synthetic: HLA-B*55:01
GSHSMRYFYTAMSRPGRGEPRFIAVGYVDDTQFVRFDSDAASPREEPRAPWIEQEGPEYWDRNTQIYKAQAQTDRESLRNLRGCYNQSEAGSHTWQTMYGCDLGPDGRLLRGHNQLAYDGKDYIALNEDLSSWTAADTAAQITQRKWEAAREAEQLRAYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 58 - protein, 276 aa, synthetic construct
  REGION 1..276: note = Synthetic: HLA-C*03:03
GSHSMRYFYTAVSRPGRGEPHFIAVGYVDDTQFVRFDSDAASPRGEPRAPWVEQEGPEYWDRETQKYKRQAQTDRVSLRNLRGCYNQSEARSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 59 - protein, 276 aa, synthetic construct
  REGION 1..276: note = Synthetic: HLA-C*07:04
CSHSMRYFDTAVSRPGRGEPRFISVGYVDDTQFVRFDSDAASPRGEPRAPWVEQEGPEYWDRETQKYKRQAQADRVSLRNLRGCYNQSEDGSHTFQRMYGCDLGPDGRLLRGYDQFAYDGKDYIALNEDLRSWTAADTAAQITQRKLEAARAAEQDRAYLEGTCVEWLRRYLENGKKTLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 60 - protein, 276 aa, synthetic construct
  REGION 1..276: note = Synthetic: HLA-C*08:01
CSHSMRYFYTAVSRPGRGEPRFIAVGYVDDTQFVQFDSDAASPRGEPRAPWVEQEGPEYWDRETQKYKRQAQTDRVSLRNLRGCYNQSEAGSHTLQRMYGCDLGPDGRLLRGYNQFAYDGKDYIALNEDLRSWTAADTAAQITQRKWEAARTAEQLRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGP

SEQ ID NO: 61 - protein, 276 aa, synthetic construct
  REGION 1..276: note = Synthetic: HLA-C*08:02
CSHSMRYFYTAVSRPGRGEPRFIAVGYVDDTQFVQFDSDAASPRGEPRAPWVEQEGPEYWDRETQKYKRQAQTDRVSLRNLRGCYNQSEAGSHTLQRMYGCDLGPDGRLLRGYNQFAYDGKDYIALNEDLRSWTAADKAAQITQRKWEAAREAEQRRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGP

SEQ ID NO: 62 - protein, 276 aa, synthetic construct
  REGION 1..276: note = Synthetic: HLA-C*12:02
CSHSMRYFYTAVSRPGRGEPRFIAVGYVDDTQFVRFDSDAASPRGEPRAPWVEQEGPEYWDRETQKYKRQAQADRVSLRNLRGCYNQSEAGSHTLQRMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 63 - protein, 276 aa, synthetic construct
  REGION 1..276: note = Synthetic: HLA-C*12:03
CSHSMRYFYTAVSRPGRGEPRFIAVGYVDDTQFVRFDSDAASPRGEPRAPWVEQEGPEYWDRETQKYKRQAQADRVSLRNLRGCYNQSEAGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 64 - protein, 276 aa, synthetic construct
  REGION 1..276: note = Synthetic: HLA-C*14:02
CSHSMRYFSTSVSRPGRGEPRFIAVGYVDDTQFVRFDSDAASPRGEPRAPWVEQEGPEYWDRETQKYKRQAQTDRVSLRNLRGCYNQSEAGSHTLQWMFGCDLGPDGRLLRGYDQSAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 65 - protein, 276 aa, synthetic construct
  REGION 1..276: note = Synthetic: HLA-C*15:02
CSHSMRYFYTAVSRPGRGEPHFIAVGYVDDTQFVRFDSDAASPRGEPRAPWVEQEGPEYWDRETQNYKRQAQTDRVNLRKLRGCYNQSEAGSHIIQRMYGCDLGPDGRLLRGHDQLAYDGKDYIALNEDLRSWTAADTAAQITQRKWEAAREAEQLRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 66 - protein, 276 aa, synthetic construct
  REGION 1..276: note = Synthetic: HLA-C*17:01
GSHSMRYFYTAVSRPGRGEPRFIAVGYVDDTQFVRFDSDAASPRGEPRAPWVEQEGPEYWDRETQKYKRQAQADRVNLRKLRGCYNQSEAGSHTIQRMYGCDLGPDGRLLRGYNQFAYDGKDYIALNEDLRSWTAADTAAQISQRKLEAAREAEQLRAYLEGECVEWLRGYLENGKETLQRAERPKTHVTHHPVSDHEATLRCWALGFYPAEITLTWQRDGEDQTQDTELVETRPAGDGTFQKWAAVVVPSGQEQRYTCHVQHEGLQEPCTLRWKP

SEQ ID NO: 67 - DNA, 828 bp, synthetic construct
  misc_feature 1..828: note = Synthetic: HLA-A*01:01
  source 1..828: mol_type = other DNA
ggctcccactccatgaggtatttcttcacatccgtgtcccggcccggccgcggggagccccgcttcatcgccgtgggctacgtggacgacacgcagttcgtgcggttcgacagcgacgccgcgagccagaagatggagccgcgggcgccgtggatagagcaggaggggccggagtattgggaccaggagacacggaatatgaaggcccactcacagactgaccgagcgaacctggggaccctgcgcggctgctacaaccagagcgaggacggttctcacaccatccagataatgtatggctgcgacgtggggccggacgggcgcttcctccgcgggtaccggcaggacgcctacgacggcaaggattacatcgccctgaacgaggacctgcgctcttggaccgcggcggacatggcagctcagatcaccaagcgcaagtgggaggcggtccatgcggcggagcagcggagagtctacctggagggccggtgcgtggacgggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctg

SEQ ID NO: 68 - DNA, 828 bp, synthetic construct
  misc_feature 1..828: note = Synthetic: HLA-A*02:01
  source 1..828: mol_type = other DNA
ggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtcccgagtattgggacggggagacacggaaagtgaaggcccactcacagactcaccgagtggacctggggaccctgcgcggctgctacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccg

SEQ ID NO: 69 - DNA, 828 bp, synthetic construct
  misc_feature 1..828: note = Synthetic: HLA-A*03:01
  source 1..828: mol_type = other DNA
ggctcccactccatgaggtatttcttcacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccaggagacacggaatgtgaaggcccagtcacagactgaccgagtggacctggggaccctgcgcggctgctacaaccagagcgaggccggttctcacaccatccagataatgtatggctgcgacgtggggtcggacgggcgcttcctccgcgggtaccggcaggacgcctacgacggcaaggattacatcgccctgaacgaggacctgcgctcttggaccgcggcggacatggcggctcagatcaccaagcgcaagtgggaggcggcccatgaggcggagcagttgagagcctacctggatggcacgtgcgtggagtggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctg

SEQ ID NO: 70 - DNA, 828 bp, synthetic construct
  misc_feature 1..828: note = Synthetic: HLA-A*24:02
  source 1..828: mol_type = other DNA
ggctcccactccatgaggtatttctccacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggacgaggagacagggaaagtgaaggcccactcacagactgaccgagagaacctgcggatcgcgctccgctgctacaaccagagcgaggccggttctcacaccctccagatgatgtttggctgcgacgtggggtcggacgggcgcttcctccgcgggtaccaccagtacgcctacgacggcaaggattacatcgccctgaaagaggacctgcgctcttggaccgcggcggacatggcggctcagatcaccaagcgcaagtgggaggcggcccatgtggcggagcagcagagagcctacctggagggcacgtgcgtggacgggctccgcagatacctggagaacgggaaggagacgctgcagcgcacggacccccccaagacacatatgacccaccaccccatctctgaccatgaggccactctgagatgctgggccctgggcttctaccctgcggagatcacactgacctggcagcgggatggggaggaccagacccaggacacggagcttgtggagaccaggcctgcaggggatggaaccttccagaagtgggcagctgtggtggtaccttctggagaggagcagagatacacctgccatgtgcagcatgagggtctgcccaagcccctcaccctgagatgggagcca

SEQ ID NO: 71 - DNA, 828 bp, synthetic construct
  misc_feature 1..828: note = Synthetic: HLA-A*30:02
  source 1..828: mol_type = other DNA
ggctcccactccatgaggtatttctccacatccgtgtcccggcccggcagtggagagccccgcttcatcgcagtgggctacgtggacgacacgcagttcgtgcggttcgacagcgacgccgcgagccagaggatggagccgcgggcgccgtggatagagcaggagaggcctgagtattgggaccaggagacacggaatgtgaaggcccactcacagactgaccgagagaacctggggaccctgcgcggctgctacaaccagagcgaggccggttctcacaccatccagataatgtatggctgcgacgtggggtcggacgggcgcttcctccgcgggtatgaacagcacgcctacgacggcaaggattacatcgccctgaacgaggacctgcgctcttggaccgcggcggacatggcggctcagatcacccagcgcaagtgggaggcggcccgtcgggcggagcagttgagagcctacctggagggcacgtgcgtggagtggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctg

SEQ ID NO: 72 - DNA, 828 bp, synthetic construct
  misc_feature 1..828: note = Synthetic: HLA-A*31:01
  source 1..828: mol_type = other DNA
ggctcccactccatgaggtatttcaccacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggagaggcctgagtattgggaccaggagacacggaatgtgaaggcccactcacagattgaccgagtggacctggggaccctgcgcggctgctacaaccagagcgaggccggttctcacaccatccagatgatgtatggctgcgacgtggggtcggacgggcgcttcctccgcgggtaccagcaggacgcctacgacggcaaggattacatcgccttgaacgaggacctgcgctcttggaccgcggcggacatggcggctcagatcacccagcgcaagtgggaggcggcccgtgtggcggagcagttgagagcctacctggagggcacgtgcgtggagtggctccgcagatacctggagaacgggaaggagacgctgcagcgcacggacccccccaagacgcatatgactcaccacgctgtctctgaccatgaggccaccctgaggtgctgggccctgagcttctaccctgcggagatcacactgacctggcagcgggatggggaggaccagacccaggacacggagctcgtggagaccaggcctgcaggggatggaaccttccagaagtgggcgtctgtggtggtgccttctggacaggagcagagatacacctgccatgtgcagcatgagggtctccccaagcccctcaccctgagatgggagccg

SEQ ID NO: 73 - DNA, 828 bp, synthetic construct
  misc_feature 1..828: note = Synthetic: HLA-A*32:01
  source 1..828: mol_type = other DNA
ggctcccactccatgaggtatttcttcacatccgtgtcccggcccggccgcggggagccccgcttcatcgccgtgggctacgtggacgacacgcagttcgtgcggtttgacagcgacgccgcgagccagaggatggagccgcgggcgccgtggatagagcaggaggggccggagtattgggaccaggagacacggaatgtgaaggcccactcacagactgaccgagagagcctgcggatcgcgctccgctgctacaaccagagcgaggccggttctcacaccatccagatgatgtatggctgcgacgtggggccggacgggcgcctcctccgcgggtaccagcaggacgcctacgacggcaaggattacatcgccttgaacgaggacctgcgctcttggaccgcggcggacatggcggctcagatcacccagcgcaagtgggaggcggcccgtgtggcggagcagttgagagcctacctggagggcacgtgcgtggagtggctccgcagatacctggagaacgggaaggagacgctgcagcgcacggacgcccccaagacgcatatgactcaccacgctgtctctgaccatgaggccaccctgaggtgctgggccctgagcttctaccctgcggagatcacactgacctggcagcgggatggggaggaccagacccaggacacggagcttgtggagaccaggcctgcaggggatggaaccttccagaagtgggcgtctgtggtggtgccttctggacaggagcagagatacacctgccatgtgcagcatgagggtctgcccaagcccctcaccctgagatgggagccg

SEQ ID NO: 74 - DNA, 828 bp, synthetic construct
  misc_feature 1..828: note = Synthetic: HLA-A*33:01
  source 1..828: mol_type = other DNA
ggctcccactccatgaggtatttcaccacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccggaacacacggaatgtgaaggcccactcacagattgaccgagtggacctggggaccctgcgcggctgctacaaccagagcgaggccggttctcacaccatccagatgatgtatggctgcgacgtggggtcggacgggcgcttcctccgcgggtaccagcaggacgcctacgacggcaaggattacatcgccttgaacgaggacctgcgctcttggaccgcggcggacatggcggctcagatcacccagcgcaagtgggaggcggcccgtgtggcggagcagttgagagcctacctggagggcacgtgcgtggagtggctccgcagacacctggagaacgggaaggagacgctgcagcgcacggacccccccaggacgcatatgactcaccacgctgtctctgaccatgaggccaccctgaggtgctgggccctgagcttctaccctgcggagatcacactgacctggcagcgggatggggaggaccagacccaggacacggagctcgtggagaccaggcctgcaggggatggaaccttccagaagtgggcgtctgtggtggtgccttctggacaggagcagagatacacctgccatgtgcagcatgagggtctccccaagcccctcaccctgagatgggagccg

SEQ ID NO: 75 - DNA, 828 bp, synthetic construct
  misc_feature 1..828: note = Synthetic: HLA-A*68:01
  source 1..828: mol_type = other DNA
ggctcccactccatgaggtatttctacacctccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccggaacacacggaatgtgaaggcccagtcacagactgaccgagtggacctggggaccctgcgcggctgctacaaccagagcgaggccggttctcacaccatccagatgatgtatggctgcgacgtggggtcggacgggcgcttcctccgcgggtaccggcaggacgcctacgacggcaaggattacatcgccctgaaagaggacctgcgctcttggaccgcggcggacatggcagctcagaccaccaagcacaagtgggaggcggcccatgtggcggagcagtggagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggtggctgtggtggtgccttctggacaggagcagagatacacctgccatgtgcagcatgagggtttgcccaagcccctcaccctgagatgggagccg

SEQ ID NO: 76 - DNA, 828 bp, synthetic construct
  misc_feature 1..828: note = Synthetic: HLA-B*07:02
  source 1..828: mol_type = other DNA
ggctcccactccatgaggtatttctacacctccgtgtcccggcccggccgcggggagccccgcttcatctcagtgggctacgtggacgacacccagttcgtgaggttcgacagcgacgccgcgagtccgagagaggagccgcgggcgccgtggatagagcaggaggggccggagtattgggaccggaacacacagatctacaaggcccaggcacagactgaccgagagagcctgcggaacctgcgcggctgctacaaccagagcgaggccgggtctcacaccctccagagcatgtacggctgcgacgtggggccggacgggcgcctcctccgcgggcatgaccagtacgcctacgacggcaaggattacatcgccctgaacgaggacctgcgctcctggaccgccgcggacacggcggctcagatcacccagcgcaagtgggaggcggcccgtgaggcggagcagcggagagcctacctggagggcgagtgcgtggagtggctccgcagatacctggagaacgggaaggacaagctggagcgcgctgaccccccaaagacacacgtgacccaccaccccatctctgaccatgaggccaccctgaggtgctgggccctgggtttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccg

SEQ ID NO: 77 - DNA, 828 bp, synthetic construct
  misc_feature 1..828: note = Synthetic: HLA-B*14:02
  source 1..828: mol_type = other DNA
ggctcccactccatgaggtatttctacaccgccgtgtcccggcccggccgcggggagccccgcttcatctcagtgggctacgtggacgacacgcagttcgtgaggttcgacagcgacgccgcgagtccgagagaggagccgcgggcgccgtggatagagcaggaggggccggaatattgggaccggaacacacagatctgcaagaccaacacacagactgaccgagagagcctgcggaacctgcgcggctgctacaaccagagcgaggccgggtctcacaccctccagtggatgtatggctgcgacgtggggccggacgggcgcctcctccgcgggtataaccagttcgcctacgacggcaaggattacatcgccctgaacgaggacctgagctcctggaccgcggcggacaccgcggctcagatcacccagcgcaagtgggaggcggcccgtgaggcggagcagctgagagcctacctggagggcacgtgcgtggagtggctccgcagacacctggagaacgggaaggagacgctgcagcgcgcggaccccccaaagacacatgtgacccaccaccccatctctgaccatgaggccaccctgaggtgctgggccctgggcttctaccctgcggagatcacactgacctggcagcgggatggcgaggaccaaactcaggacaccgagcttgtggagaccagaccagcaggagacagaaccttccagaagtgggcagctgtggtggtgccttctggagaagagcagagatacacatgccatgtacagcatgaggggctgccgaagcccctcaccctgagatgggagcca

SEQ ID NO: 78 - DNA, 828 bp, synthetic construct
  misc_feature 1..828: note = Synthetic: HLA-B*18:01
  source 1..828: mol_type = other DNA
ggctcccactccatgaggtatttccacacctccgtgtcccggcccggccgcggggagccccgcttcatctcagtgggctacgtggacggcacccagttcgtgaggttcgacagcgacgccgcgagtccgaggacggagccccgggcgccgtggatagagcaagaggggccggagtattgggaccggaacacacagatctccaagaccaacacacagacttaccgagagagcctgcggaacctgcgcggctgctacaaccagagcgaggccgggtctcacaccctccagaggatgtacggctgcgacgtggggccggacgggcgcctcctccgcgggcatgaccagtccgcctacgacggcaaggattacatcgccctgaacgaggacctgagctcctggaccgcggcggacaccgcggctcagatcacccagcgcaagtgggaggcggcccgtgtggcggagcagctgagagcctacctggagggcacgtgcgtggagtggctccgcagacacctggagaacgggaaggagacgctgcagcgcgcggaccccccaaagacacatgtgacccaccaccccatctctgaccatgaggccaccctgaggtgctgggccctgggcttctaccctgcggagatcacactgacctggcagcgggatggcgaggaccaaactcaggacaccgagcttgtggagaccagaccagcaggagatagaaccttccagaagtgggcagctgtggtggtgccttctggagaagagcagagatacacatgccatgtacagcatgaggggctgccgaagcccctcaccctgagatgggagcca

SEQ ID NO: 79 - DNA, 828 bp, synthetic construct
  misc_feature 1..828: note = Synthetic: HLA-B*27:02
  source 1..828: mol_type = other DNA
ggctcccactccatgaggtatttccacacctccgtgtcccggcccggccgcggggagccccgcttcatcaccgtgggctacgtggacgacacgctgttcgtgaggttcgacagcgacgccgcgagtccgagagaggagccgcgggcgccgtggatagagcaggaggggccggagtattgggaccgggagacacagatctgcaaggccaaggcacagactgaccgagagaacctgcggatcgcgctccgctgctacaaccagagcgaggccgggtctcacaccctccagaatatgtatggctgcgacgtggggccggacgggcgcctcctccgcgggtaccaccaggacgcctacgacggcaaggattacatcgccctgaacgaggacctgagctcctggaccgccgcggacacggcggctcagatcacccagcgcaagtgggaggcggcccgtgtggcggagcagctgagagcctacctggagggcgagtgcgtggagtggctccgcagatacctggagaacgggaaggagacgctgcagcgcgcggaccccccaaagacacacgtgacccaccaccccatctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccg

SEQ ID NO: 80 - DNA, 828 bp, synthetic construct
  misc_feature 1..828: note = Synthetic: HLA-B*39:01
  source 1..828: mol_type = other DNA
ggctcccactccatgaggtatttctacacctccgtgtcccggcccggccgcggggagccccgcttcatctcagtgggctacgtggacgacacgcagttcgtgaggttcgacagcgacgccgcgagtccgagagaggagccgcgggcgccgtggatagagcaggaggggccggaatattgggaccggaacacacagatctgcaagaccaacacacagactgaccgagagagcctgcggaacctgcgcggctgctacaaccagagcgaggccgggtctcacaccctccagaggatgtacggctgcgacgtggggccggacgggcgcctcctccgcgggcataaccagttcgcctacgacggcaaggattacatcgccctgaacgaggacctgagctcctggaccgcggcggacaccgcggctcagatcacccagcgcaagtgggaggcggcccgtgtggcggagcagctgagaacctacctggagggcacgtgcgtggagtggctccgcagatacctggagaacgggaaggagacgctgcagcgcgcggaccccccaaagacacatgtgacccaccaccccatctctgaccatgaggccaccctgaggtgctgggccctgggcttctaccctgcggagatcacactgacctggcagcgggatggcgaggaccaaactcaggacaccgagcttgtggagaccagaccagcaggagacagaaccttccagaagtgggcagctgtggtggtgccttctggagaagagcagagatacacatgccatgtacagcatgaggggctgccgaagcccctcaccctgagatgggagcca

SEQ ID NO: 81 - DNA, 828 bp, synthetic construct
  misc_feature 1..828: note = Synthetic: HLA-B*40:01
  source 1..828: mol_type = other DNA
ggctcccactccatgaggtatttccacaccgccatgtcccggcccggccgcggggagccccgcttcatcaccgtgggctacgtggacgacacgctgttcgtgaggttcgacagcgacgccacgagtccgaggaaggagccgcgggcgccatggatagagcaggaggggccggagtattgggaccgggagacacagatctccaagaccaacacacagacttaccgagagagcctgcggaacctgcgcggctgctacaaccagagcgaggccgggtctcacaccctccagaggatgtacggctgcgacgtggggccggacgggcgcctcctccgcgggcataaccagtacgcctacgacggcaaggattacatcgccctgaacgaggacctgcgctcctggaccgccgcggacacggcggctcagatctcccagcgcaagttggaggcggcccgtgtggcggagcagctgagagcctacctggagggcgagtgcgtggagtggctccgcagatacctggagaacgggaaggacaagctggagcgcgctgaccccccaaagacacacgtgacccaccaccccatctctgaccatgaggccaccctgaggtgctgggccctgggtttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccg

SEQ ID NO: 82 - DNA, 828 bp, synthetic construct
  misc_feature 1..828: note = Synthetic: HLA-B*44:02
  source 1..828: mol_type = other DNA
ggctcccactccatgaggtatttctacaccgccatgtcccggcccggccgcggggagccccgcttcatcaccgtgggctacgtggacgacacgctgttcgtgaggttcgacagcgacgccacgagtccgaggaaggagccgcgggcgccatggatagagcaggaggggccggagtattgggaccgggagacacagatctccaagaccaacacacagacttaccgagagaacctgcgcaccgcgctccgctgctacaaccagagcgaggccgggtctcacatcatccagaggatgtacggctgcgacgtggggccggacgggcgcctcctccgcgggtatgaccaggacgcctacgacggcaaggattacatcgccctgaacgaggacctgagctcctggaccgcggcggacaccgcggctcagatcacccagcgcaagtgggaggcggcccgtgtggcggagcaggacagagcctacctggagggcctgtgcgtggagtcgctccgcagatacctggagaacgggaaggagacgctgcagcgcgcggaccccccaaagacacatgtgacccaccaccccatctctgaccatgaggtcaccctgaggtgctgggccctgggcttctaccctgcggagatcacactgacctggcagcgggatggcgaggaccaaactcaggacaccgagcttgtggagaccagaccagcaggagatagaaccttccagaagtgggcagctgtggtggtgccttctggagaagagcagagatacacatgccatgtacagcatgaggggctgccgaagcccctcaccctgagatgggagccg

SEQ ID NO: 83 - DNA, 828 bp, synthetic construct
  misc_feature 1..828: note = Synthetic: HLA-B*46:01
  source 1..828: mol_type = other DNA
ggctcccactccatgaggtatttctacaccgccatgtcccggcccggccgcggggagccccgcttcatcgcagtgggctacgtggacgacacccagttcgtgaggttcgacagcgacgccgcgagtccgaggatggcgccccgggcgccatggatagagcaggaggggccggagtattgggaccgggagacacagaagtacaagcgccaggcacagactgaccgagtgagcctgcggaacctgcgcggctgctacaaccagagcgaggccgggtctcacaccctccagaggatgtacggctgcgacgtggggccggacgggcgcctcctccgcgggcatgaccagtccgcctacgacggcaaggattacatcgccctgaacgaggacctgagctcctggaccgcggcggacacggcggctcagatcacccagcgcaagtgggaggcggcccgtgaggcggagcagtggagagcctacctggagggcctgtgcgtggagtggctccgcagatacctggagaacgggaaggagacgctgcagcgcgcggaccccccaaagacacatgtgacccaccaccccatctctgaccatgaggccaccctgaggtgctgggccctgggcttctaccctgcggagatcacactgacctggcagcgggatggcgaggaccaaactcaggacaccgagcttgtggagaccagaccagcaggagatagaaccttccagaagtgggcagctgtggtggtgccttctggagaagagcagagatacacatgccatgtacagcatgaggggctgccgaagcccctcaccctgagatgggagcca

SEQ ID NO: 84 - DNA, 828 bp, synthetic construct
  misc_feature 1..828: note = Synthetic: HLA-B*50:01
  source 1..828: mol_type = other DNA
ggctcccactccatgaggtatttccacaccgccatgtcccggcccggccgcggggagccccgcttcatcaccgtgggctacgtggacgacacgctgttcgtgaggttcgacagcgacgccacgagtccgaggaaggagccgcgggcgccatggatagagcaggaggggccggagtattgggaccgggagacacagatctccaagaccaacacacagacttaccgagagagcctgcggaacctgcgcggctgctacaaccagagcgaggccgggtctcacacttggcagaggatgtatggctgcgacctggggcccgacgggcgcctcctccgcgggtataaccagttagcctacgacggcaaggattacatcgccctgaacgaggacctgagctcctggaccgcggcggacaccgcggctcagatcacccagcgcaagtgggaggcggcccgtgaggcggagcagctgagagcctacctggagggcctgtgcgtggagtggctccgcagatacctggagaacgggaaggagacgctgcagcgcgcggaccccccaaagacacatgtgacccaccaccccatctctgaccatgaggccaccctgaggtgctgggccctgggcttctaccctgcggagatcacactgacctggcagcgggatggcgaggaccaaactcaggacaccgagcttgtggagaccagaccagcaggagatagaaccttccagaagtgggcagctgtggtggtgccttctggagaagagcagagatacacatgccatgtacagcatgaggggctgccgaagcccctcaccctgagatgggagcca

SEQ ID NO: 85 - DNA, 828 bp, synthetic construct
  misc_feature 1..828: note = Synthetic: HLA-B*57:01
  source 1..828: mol_type = other DNA
ggctcccactccatgaggtatttctacaccgccatgtcccggcccggccgcggggagccccgcttcatcgcagtgggctacgtggacgacacccagttcgtgaggttcgacagcgacgccgcgagtccgaggatggcgccccgggcgccatggatagagcaggaggggccggagtattgggacggggagacacggaacatgaaggcctccgcgcagacttaccgagagaacctgcggatcgcgctccgctgctacaaccagagcgaggccgggtctcacatcatccaggtgatgtatggctgcgacgtggggccggacgggcgcctcctccgcgggcatgaccagtccgcctacgacggcaaggattacatcgccctgaacgaggacctgagctcctggaccgcggcggacacggcggctcagatcacccagcgcaagtgggaggcggcccgtgtggcggagcagctgagagcctacctggagggcctgtgcgtggagtggctccgcagatacctggagaacgggaaggagacgctgcagcgcgcggaccccccaaagacacatgtgacccaccaccccatctctgaccatgaggccaccctgaggtgctgggccctgggcttctaccctgcggagatcacactgacctggcagcgggatggcgaggaccaaactcaggacaccgagcttgtggagaccagaccagcaggagatagaaccttccagaagtgggcagctgtggtggtgccttctggagaagagcagagatacacatgccatgtacagcatgaggggctgccaaagcccctcaccctgagatgggagcca

SEQ ID NO: 86 - DNA, 828 bp, synthetic construct
  misc_feature 1..828: note = Synthetic: HLA-B*58:01
  source 1..828: mol_type = other DNA
ggctcccactccatgaggtatttctacaccgccatgtcccggcccggccgcggggagccccgcttcatcgcagtgggctacgtggacgacacccagttcgtgaggttcgacagcgacgccgcgagtccgaggacggagccccgggcgccatggatagagcaggaggggccggagtattgggacggggagacacggaacatgaaggcctccgcgcagacttaccgagagaacctgcggatcgcgctccgctgctacaaccagagcgaggccgggtctcacatcatccagaggatgtatggctgcgacctggggcccgacgggcgcctcctccgcgggcatgaccagtccgcctacgacggcaaggattacatcgccctgaacgaggacctgagctcctggaccgcggcggacaccgcggctcagatcacccagcgcaagtgggaggcggcccgtgtggcggagcagctgagagcctacctggagggcctgtgcgtggagtggctccgcagatacctggagaacgggaaggagacgctgcagcgcgcggaccccccaaagacacacgtgacccaccaccccgtctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagcca

SEQ ID NO: 87 - DNA, 828 bp, synthetic construct
  misc_feature 1..828: note = Synthetic: HLA-C*02:02
  source 1..828: mol_type = other DNA
tgctcccactccatgaggtatttctacaccgctgtgtcccggcccagccgcggagagccccacttcatcgcagtgggctacgtggacgacacgcagttcgtgcggttcgacagcgacgccgcgagtccaagaggggagccgcgggcgccgtgggtggagcaggaggggccggagtattgggaccgggagacacagaagtacaagcgccaggcacagactgaccgagtgaacctgcggaaactacgcggctgctacaaccagagcgaggccgggtctcacaccctccagaggatgtacggctgcgacctggggcccgacgggcgcctcctccgcgggtatgaccagtccgcctacgacggcaaggattacatcgccctgaacgaggacctgcgctcctggaccgccgcggacacagcggctcagatcacccagcgcaagtgggaggcggcccgtgaggcggagcagtggagagcctacctggagggcgagtgcgtggagtggctccgcagatacctggagaacgggaaggagacgctgcagcgcgcggaacacccaaagacacacgtgacccaccatcccgtctctgaccatgaggccaccctgaggtgctgggccctgggcttctaccctacggagatcacactgacctggcagcgggatggcgaggaccaaactcaggacaccgagcttgtggagaccaggccagcaggagatggaaccttccagaagtgggcagctgtggtggtgccttctggagaagagcagagatacacgtgccatgtgcagcacgaggggctgccggagcccctcaccctgagatgggagcca

SEQ ID NO: 88 - DNA, 828 bp, synthetic construct
  misc_feature 1..828: note = Synthetic: HLA-C*03:04
  source 1..828: mol_type = other DNA
ggctcccactccatgaggtatttctacaccgctgtgtcccggcccggccgcggggagccccacttcatcgcagtgggctacgtggacgacacgcagttcgtgcggttcgacagcgacgccgcgagtccgagaggggagccgcgggcgccgtgggtggagcaggaggggccggagtattgggaccgggagacacagaagtacaagcgccaggcacagactgaccgagtgagcctgcggaacctgcgcggctgctacaaccagagcgaggccgggtctcacatcatccagaggatgtatggctgcgacgtggggcccgacgggcgcctcctccgcgggtatgaccagtacgcctacgacggcaaggattacatcgccctgaacgaggatctgcgctcctggaccgccgcggacacggcggctcagatcacccagcgcaagtgggaggcggcccgtgaggcggagcagctgagagcctacctggagggcctgtgcgtggagtggctccgcagatacctgaagaatgggaaggagacgctgcagcgcgcggaacacccaaagacacacgtgacccaccatcccgtctctgaccatgaggccaccctgaggtgctgggccctgggcttctaccctgcggagatcacactgacctggcagtgggatggggaggaccaaactcaggacactgagcttgtggagaccaggccagcaggagatggaaccttccagaagtgggcagctgtggtggtgccttctggagaagagcagagatacacgtgccatgtgcagcacgaggggctgccggagcccctcaccctgagatgggagccg

SEQ ID NO: 89 - DNA, 828 bp, synthetic construct
  misc_feature 1..828: note = Synthetic: HLA-C*05:01
  source 1..828: mol_type = other DNA
tgctcccactccatgaggtatttctacaccgccgtgtcccggcccggccgcggagagccccgcttcatcgcagtgggctacgtggacgacacgcagttcgtgcagttcgacagcgacgccgcgagtccaagaggggagccgcgggcgccgtgggtggagcaggaggggccggagtattgggaccgggagacacagaagtacaagcgccaggcacagactgaccgagtgaacctgcggaaactgcgcggctgctacaaccagagcgaggccgggtctcacaccctccagaggatgtatggctgcgacctggggcccgacgggcgcctcctccgcgggtataaccagttcgcctacgacggcaaggattacatcgccctgaatgaggacctgcgctcctggaccgccgcggacaaggcggctcagatcacccagcgcaagtgggaggcggcccgtgaggcggagcagcggagagcctacctggagggcacgtgcgtggagtggctccgcagatacctggagaacgggaagaagacgctgcagcgcgcggaacacccaaagacacacgtgacccaccatcccgtctctgaccatgaggccaccctgaggtgctgggccctgggcttctaccctgcggagatcacactgacctggcagcgggatggcgaggaccaaactcaggacaccgagcttgtggagaccaggccagcaggagatggaaccttccagaagtgggcagctgtggtggtgccttctggagaagagcagagatacacgtgccatgtgcagcacgaggggctgccagagcccctcaccctgagatgggggcca

SEQ ID NO: 90 - DNA, 828 bp, synthetic construct
  misc_feature 1..828: note = Synthetic: HLA-C*07:01
  source 1..828: mol_type = other DNA
tgctcccactccatgaggtatttcgacaccgccgtgtcccggcccggccgcggagagccccgcttcatctcagtgggctacgtggacgacacgcagttcgtgcggttcgacagcgacgccgcgagtccgagaggggagccgcgggcgccgtgggtggagcaggaggggccggagtattgggaccgggagacacagaactacaagcgccaggcacaggctgaccgagtgagcctgcggaacctgcgcggctgctacaaccagagcgaggacgggtctcacaccctccagaggatgtatggctgcgacctggggcccgacgggcgcctcctccgcgggtatgaccagtccgcctacgacggcaaggattacatcgccctgaacgaggacctgcgctcctggaccgccgcggacaccgcggctcagatcacccagcgcaagttggaggcggcccgtgcggcggagcagctgagagcctacctggagggcacgtgcgtggagtggctccgcagatacctggagaacgggaaggagacgctgcagcgcgcagaacccccaaagacacacgtgacccaccaccccctctctgaccatgaggccaccctgaggtgctgggccctgggcttctaccctgcggagatcacactgacctggcagcgggatggggaggaccagacccaggacaccgagcttgtggagaccaggccagcaggagatggaaccttccagaagtgggcagctgtggtggtgccttctggacaagagcagagatacacgtgccatatgcagcacgaggggctgcaagagcccctcaccctgagctgggagcca

SEQ ID NO: 91 - DNA, 828 bp, synthetic construct
  misc_feature 1..828: note = Synthetic: HLA-A*11:01
  source 1..828: mol_type = other DNA
ggctcccactccatgaggtatttctacacctccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccaggagacacggaatgtgaaggcccagtcacagactgaccgagtggacctggggaccctgcgcggctgctacaaccagagcgaggacggttctcacaccatccagataatgtatggctgcgacgtggggccggacgggcgcttcctccgcgggtaccggcaggacgcctacgacggcaaggattacatcgccctgaacgaggacctgcgctcttggaccgcggcggacatggcagctcagatcaccaagcgcaagtgggaggcggcccatgcggcggagcagcagagagcctacctggagggccggtgcgtggagtggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctg

SEQ ID NO: 92 - DNA, 828 bp, synthetic construct
  misc_feature 1..828: note = Synthetic: HLA-A*23:01
  source 1..828: mol_type = other DNA
ggctcccactccatgaggtatttctccacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggacgaggagacagggaaagtgaaggcccactcacagactgaccgagagaacctgcggatcgcgctccgctgctacaaccagagcgaggccggttctcacaccctccagatgatgtttggctgcgacgtggggtcggacgggcgcttcctccgcgggtaccaccagtacgcctacgacggcaaggattacatcgccctgaaagaggacctgcgctcttggaccgcggcggacatggcggctcagatcacccagcgcaagtgggaggcggcccgtgtggcggagcagttgagagcctacctggagggcacgtgcgtggacgggctccgcagatacctggagaacgggaaggagacgctgcagcgcacggacccccccaagacacatatgacccaccaccccatctctgaccatgaggccactctgagatgctgggccctgggcttctaccctgcggagatcacactgacctggcagcgggatggggaggaccagacccaggacacggagcttgtggagaccaggcctgcaggggatggaaccttccagaagtgggcagctgtggtggtaccttctggagaggagcagagatacacctgccatgtgcagcatgagggtctgcccaagcccctcaccctgagatgggagcca

SEQ ID NO: 93 - DNA, 828 bp, synthetic construct
  misc_feature 1..828: note = Synthetic: HLA-A*30:01
  source 1..828: mol_type = other DNA
ggctcccactccatgaggtatttctccacatccgtgtcccggcccggcagtggagagccccgcttcatcgcagtgggctacgtggacgacacgcagttcgtgcggttcgacagcgacgccgcgagccagaggatggagccgcgggcgccgtggatagagcaggagaggcctgagtattgggaccaggagacacggaatgtgaaggcccagtcacagactgaccgagtggacctggggaccctgcgcggctgctacaaccagagcgaggccggttctcacaccatccagataatgtatggctgcgacgtggggtcggacgggcgcttcctccgcgggtatgaacagcacgcctacgacggcaaggattacatcgccctgaacgaggacctgcgctcttggaccgcggcggacatggcggctcagatcacccagcgcaagtgggaggcggcccgttgggcggagcagttgagagcctacctggagggcacgtgcgtggagtggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctg

SEQ ID NO: 94 - DNA, 828 bp, synthetic construct
  misc_feature 1..828: note = Synthetic: HLA-A*33:03
  source 1..828: mol_type = other DNA
ggctcccactccatgaggtatttcaccacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccggaacacacggaatgtgaaggcccactcacagattgaccgagtggacctggggaccctgcgcggctgctacaaccagagcgaggccggttctcacaccatccagatgatgtatggctgcgacgtggggtcggacgggcgcttcctccgcgggtaccagcaggacgcctacgacggcaaggattacatcgccttgaacgaggacctgcgctcttggaccgcggcggacatggcggctcagatcacccagcgcaagtgggaggcggcccgtgtggcggagcagttgagagcctacctggagggcacgtgcgtggagtggctccgcagatacctggagaacgggaaggagacgctgcagcgcacggacccccccaagacgcatatgactcaccacgctgtctctgaccatgaggccaccctgaggtgctgggccctgagcttctaccctgcggagatcacactgacctggcagcgggatggggaggaccagacccaggacacggagctcgtggagaccaggcctgcaggggatggaaccttccagaagtgggcgtctgtggtggtgccttctggacaggagcagagatacacctgccatgtgcagcatgagggtctccccaagcccctcaccctgagatgggagccg

SEQ ID NO: 95 - DNA, 828 bp, synthetic construct
  misc_feature 1..828: note = Synthetic: HLA-B*08:01
  source 1..828: mol_type = other DNA
ggctcccactccatgaggtatttcgacaccgccatgtcccggcccggccgcggggagccccgcttcatctcagtgggctacgtggacgacacgcagttcgtgaggttcgacagcgacgccgcgagtccgagagaggagccgcgggcgccgtggatagagcaggaggggccggagtattgggaccggaacacacagatcttcaagaccaacacacagactgaccgagagagcctgcggaacctgcgcggctgctacaaccagagcgaggccgggtctcacaccctccagagcatgtacggctgcgacgtggggccggacgggcgcctcctccgcgggcataaccagtacgcctacgacggcaaggattacatcgccctgaacgaggacctgcgctcctggaccgcggcggacaccgcggctcagatcacccagcgcaagtgggaggcggcccgtgtggcggagcaggacagagcctacctggagggcacgtgcgtggagtggctccgcagatacctggagaacgggaaggacacgctggagcgcgcggaccccccaaagacacacgtgacccaccaccccatctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccg

SEQ ID NO: 96 - DNA, 828 bp, synthetic construct
  misc_feature 1..828: note = Synthetic: HLA-B*15:01
  source 1..828: mol_type = other DNA
ggctcccactccatgaggtatttctacaccgccatgtcccggcccggccgcggggagccccgcttcatcgcagtgggctacgtggacgacacccagttcgtgaggttcgacagcgacgccgcgagtccgaggatggcgccccgggcgccatggatagagcaggaggggccggagtattgggaccgggagacacagatctccaagaccaacacacagacttaccgagagagcctgcggaacctgcgcggctgctacaaccagagcgaggccgggtctcacaccctccagaggatgtacggctgcgacgtggggccggacgggcgcctcctccgcgggcatgaccagtccgcctacgacggcaaggattacatcgccctgaacgaggacctgagctcctggaccgcggcggacacggcggctcagatcacccagcgcaagtgggaggcggcccgtgaggcggagcagtggagagcctacctggagggcctgtgcgtggagtggctccgcagatacctggagaacgggaaggagacgctgcagcgcgcggaccccccaaagacacatgtgacccaccaccccatctctgaccatgaggccaccctgaggtgctgggccctgggcttctaccctgcggagatcacactgacctggcagcgggatggcgaggaccaaactcaggacaccgagcttgtggagaccagaccagcaggagatagaaccttccagaagtgggcagctgtggtggtgccttctggagaagagcagagatacacatgccatgtacagcatgaggggctgccgaagcccctcaccctgagatgggagcca

SEQ ID NO: 97 - DNA, 828 bp, synthetic construct
  misc_feature 1..828: note = Synthetic: HLA-B*15:03
  source 1..828: mol_type = other DNA
ggctcccactccatgaggtatttctacaccgccatgtcccggcccggccgcggggagccccgcttcatctcagtgggctacgtggacgacacgcagttcgtgaggttcgacagcgacgccgcgagtccgagagaggagccgcgggcgccgtggatagagcaggaggggccggagtattgggaccgggagacacagatctccaagaccaacacacagacttaccgagagagcctgcggaacctgcgcggctgctacaaccagagcgaggccgggtctcacaccctccagaggatgtacggctgcgacgtggggccggacgggcgcctcctccgcgggcatgaccagtccgcctacgacggcaaggattacatcgccctgaacgaggacctgagctcctggaccgcggcggacacggcggctcagatcacccagcgcaagtgggaggcggcccgtgaggcggagcagctgagagcctacctggagggcctgtgcgtggagtggctccgcagatacctggagaacgggaaggagacgctgcagcgcgcggaccccccaaagacacatgtgacccaccaccccatctctgaccatgaggccaccctgaggtgctgggccctgggcttctaccctgcggagatcacactgacctggcagcgggatggcgaggaccaaactcaggacaccgagcttgtggagaccagaccagcaggagatagaaccttccagaagtgggcagctgtggtggtgccttctggagaagagcagagatacacatgccatgtacagcatgaggggctgccgaagcccctcaccctgagatgggagcca

SEQ ID NO: 98 - DNA, 828 bp, synthetic construct
  misc_feature 1..828: note = Synthetic: HLA-B*35:01
  source 1..828: mol_type = other DNA
ggctcccactccatgaggtatttctacaccgccatgtcccggcccggccgcggggagccccgcttcatcgcagtgggctacgtggacgacacccagttcgtgaggttcgacagcgacgccgcgagtccgaggacggagccccgggcgccatggatagagcaggaggggccggagtattgggaccggaacacacagatcttcaagaccaacacacagacttaccgagagagcctgcggaacctgcgcggctgctacaaccagagcgaggccgggtctcacatcatccagaggatgtatggctgcgacctggggcccgacgggcgcctcctccgcgggcatgaccagtccgcctacgacggcaaggattacatcgccctgaacgaggacctgagctcctggaccgcggcggacaccgcggctcagatcacccagcgcaagtgggaggcggcccgtgtggcggagcagctgagagcctacctggagggcctgtgcgtggagtggctccgcagatacctggagaacgggaaggagacgctgcagcgcgcggaccccccaaagacacacgtgacccaccaccccgtctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagcca

SEQ ID NO: 99 - DNA, 828 bp, synthetic construct
  misc_feature 1..828: note = Synthetic: HLA-B*40:02
  source 1..828: mol_type = other DNA
ggctcccactccatgaggtatttccacacctccgtgtcccggcccggccgcggggagccccgcttcatcaccgtgggctacgtggacgacacgctgttcgtgaggttcgacagcgacgccacgagtccgaggaaggagccgcgggcgccatggatagagcaggaggggccggagtattgggaccgggagacacagatctccaagaccaacacacagacttaccgagagagcctgcggaacctgcgcggctgctacaaccagagcgaggccgggtctcacaccctccagagcatgtacggctgcgacgtggggccggacgggcgcctcctccgcgggcataaccagtacgcctacgacggcaaggattacatcgccctgaacgaggacctgcgctcctggaccgccgcggacacggcggctcagatcacccagcgcaagtgggaggcggcccgtgtggcggagcagctgagagcctacctggagggcgagtgcgtggagtggctccgcagatacctggagaacgggaaggagacgctgcagcgcgcggaccccccaaagacacacgtgacccaccaccccatctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccg

SEQ ID NO: 100 - DNA, 828 bp, synthetic construct
  misc_feature 1..828: note = Synthetic: HLA-B*42:01
  source 1..828: mol_type = other DNA
ggctcccactccatgaggtatttctacacctccgtgtcccggcccggccgcggggagccccgcttcatctcagtgggctacgtggacgacacccagttcgtgaggttcgacagcgacgccgcgagtccgagagaggagccgcgggcgccgtggatagagcaggaggggccggagtattgggaccggaacacacagatctacaaggcccaggcacagactgaccgagagagcctgcggaacctgcgcggctgctacaaccagagcgaggccgggtctcacaccctccagagcatgtacggctgcgacgtggggccggacgggcgcctcctccgcgggcataaccagtacgcctacgacggcaaggattacatcgccctgaacgaggacctgcgctcctggaccgcggcggacaccgcggctcagatcacccagcgcaagtgggaggcggcccgtgtggcggagcaggacagagcctacctggagggcacgtgcgtggagtggctccgcagatacctggagaacgggaaggacacgctggagcgcgcggaccccccaaagacacacgtgacccaccaccccatctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccg

SEQ ID NO: 101 - DNA, 828 bp, synthetic construct
  misc_feature 1..828: note = Synthetic: HLA-B*44:03
  source 1..828: mol_type = other DNA
ggctcccactccatgaggtatttctacaccgccatgtcccggcccggccgcggggagccccgcttcatcaccgtgggctacgtggacgacacgctgttcgtgaggttcgacagcgacgccacgagtccgaggaaggagccgcgggcgccatggatagagcaggaggggccggagtattgggaccgggagacacagatctccaagaccaacacacagacttaccgagagaacctgcgcaccgcgctccgctgctacaaccagagcgaggccgggtctcacatcatccagaggatgtacggctgcgacgtggggccggacgggcgcctcctccgcgggtatgaccaggacgcctacgacggcaaggattacatcgccctgaacgaggacctgagctcctggaccgcggcggacaccgcggctcagatcacccagcgcaagtgggaggcggcccgtgtggcggagcagctgagagcctacctggagggcctgtgcgtggagtcgctccgcagatacctggagaacgggaaggagacgctgcagcgcgcggaccccccaaagacacatgtgacccaccaccccatctctgaccatgaggtcaccctgaggtgctgggccctgggcttctaccctgcggagatcacactgacctggcagcgggatggcgaggaccaaactcaggacaccgagcttgtggagaccagaccagcaggagatagaaccttccagaagtgggcagctgtggtggtgccttctggagaagagcagagatacacatgccatgtacagcatgaggggctgccgaagcccctcaccctgagatgggagccg

SEQ ID NO: 102 - DNA, 828 bp, synthetic construct
  misc_feature 1..828: note = Synthetic: HLA-B*51:01
  source 1..828: mol_type = other DNA
ggctcccactccatgaggtatttctacaccgccatgtcccggcccggccgcggggagccccgcttcattgcagtgggctacgtggacgacacccagttcgtgaggttcgacagcgacgccgcgagtccgaggacggagccccgggcgccatggatagagcaggaggggccggagtattgggaccggaacacacagatcttcaagaccaacacacagacttaccgagagaacctgcggatcgcgctccgctgctacaaccagagcgaggccgggtctcacacttggcagacgatgtatggctgcgacgtggggccggacgggcgcctcctccgcgggcataaccagtacgcctacgacggcaaagattacatcgccctgaacgaggacctgagctcctggaccgcggcggacaccgcggctcagatcacccagcgcaagtgggaggcggcccgtgaggcggagcagctgagagcctacctggagggcctgtgcgtggagtggctccgcagacacctggagaacgggaaggagacgctgcagcgcgcggaccccccaaagacacacgtgacccaccaccccgtctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagcca

SEQ ID NO: 103 - DNA, 828 bp, synthetic construct
  misc_feature 1..828: note = Synthetic: HLA-B*53:01
  source 1..828: mol_type = other DNA
ggctcccactccatgaggtatttctacaccgccatgtcccggcccggccgcggggagccccgcttcatcgcagtgggctacgtggacgacacccagttcgtgaggttcgacagcgacgccgcgagtccgaggacggagccccgggcgccatggatagagcaggaggggccggagtattgggaccggaacacacagatcttcaagaccaacacacagacttaccgagagaacctgcggatcgcgctccgctgctacaaccagagcgaggccgggtctcacatcatccagaggatgtatggctgcgacctggggcccgacgggcgcctcctccgcgggcatgaccagtccgcctacgacggcaaggattacatcgccctgaacgaggacctgagctcctggaccgcggcggacaccgcggctcagatcacccagcgcaagtgggaggcggcccgtgtggcggagcagctgagagcctacctggagggcctgtgcgtggagtggctccgcagatacctggagaacgggaaggagacgctgcagcgcgcggaccccccaaagacacacgtgacccaccaccccgtctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagcca

SEQ ID NO: 104 - DNA, 828 bp, synthetic construct
  misc_feature 1..828: note = Synthetic: HLA-C*01:02
  source 1..828: mol_type = other DNA
tgctcccactccatgaagtatttcttcacatccgtgtcccggcctggccgcggagagccccgcttcatctcagtgggctacgtggacgacacgcagttcgtgcggttcgacagcgacgccgcgagtccgagaggggagccgcgggcgccgtgggtggagcaggaggggccggagtattgggaccgggagacacagaagtacaagcgccaggcacagactgaccgagtgagcctgcggaacctgcgcggctgctacaaccagagcgaggccgggtctcacaccctccagtggatgtgtggctgcgacctggggcccgacgggcgcctcctccgcgggtatgaccagtacgcctacgacggcaaggattacatcgccctgaacgaggacctgcgctcctggaccgccgcggacaccgcggctcagatcacccagcgcaagtgggaggcggcccgtgaggcggagcagcggagagcctacctggagggcacgtgcgtggagtggctccgcagatacctggagaacgggaaggagacgctgcagcgcgcggaacacccaaagacacacgtgacccaccatcccgtctctgaccatgaggccaccctgaggtgctgggccctgggcttctaccctgcggagatcacactgacctggcagtgggatggggaggaccaaactcaggacaccgagcttgtggagaccaggccagcaggagatggaaccttccagaagtgggcagctgtgatggtgccttctggagaagagcagagatacacgtgccatgtgcagcacgaggggctgccggagcccctcaccctgagatgggagccg

SEQ ID NO: 105 - DNA, 828 bp, synthetic construct
  misc_feature 1..828: note = Synthetic: HLA-C*04:01
  source 1..828: mol_type = other DNA
ggctcccactccatgaggtatttctccacatccgtgtcctggcccggccgcggggagccccgcttcatcgcagtgggctacgtggacgacacgcagttcgtgcggttcgacagcgacgccgcgagtccaagaggggagccgcgggagccgtgggtggagcaggaggggccggagtattgggaccgggagacacagaagtacaagcgccaggcacaggctgaccgagtgaacctgcggaaactgcgcggctgctacaaccagagcgaggacgggtctcacaccctccagaggatgtttggctgcgacctggggccggacgggcgcctcctccgcgggtataaccagttcgcctacgacggcaaggattacatcgccctgaacgaggatctgcgctcctggaccgccgcggacacggcggctcagatcacccagcgcaagtgggaggcggcccgtgaggcggagcagcggagagcctacctggagggcacgtgcgtggagtggctccgcagatacctggagaacgggaaggagacgctgcagcgcgcggaacacccaaagacacacgtgacccaccatcccgtctctgaccatgaggccaccctgaggtgctgggccctgggcttctaccctgcggagatcacactgacctggcagtgggatggggaggaccaaactcaggacaccgagcttgtggagaccaggccagcaggagatggaaccttccagaagtgggcagctgtggtggtgccttctggagaagagcagagatacacgtgccatgttcagcacgaggggctgccggagcccctcaccctgagatggaagccg

SEQ ID NO: 106 - DNA, 828 bp, synthetic construct
  misc_feature 1..828: note = Synthetic: HLA-C*06:02
  source 1..828: mol_type = other DNA
tgctcccactccatgaggtatttcgacaccgccgtgtcccggcccggccgcggagagccccgcttcatctcagtgggctacgtggacgacacgcagttcgtgcggttcgacagcgacgccgcgagtccgagaggggagccccgggcgccgtgggtggagcaggaggggccggagtattgggaccgggagacacagaagtacaagcgccaggcacaggctgaccgagtgaacctgcggaaactgcgcggctgctacaaccagagcgaggacgggtctcacaccctccagtggatgtatggctgcgacctggggcccgacgggcgcctcctccgcgggtatgaccagtccgcctacgacggcaaggattacatcgccctgaacgaggacctgcgctcctggaccgccgcggacacggcggctcagatcacccagcgcaagtgggaggcggcccgtgaggcggagcagtggagagcctacctggagggcacgtgcgtggagtggctccgcagatacctggagaacgggaaggagacgctgcagcgcgcggaacacccaaagacacacgtgacccaccatcccgtctctgaccatgaggccaccctgaggtgctgggccctgggcttctaccctgcggagatcacactgacctggcagcgggatggcgaggaccaaactcaggacaccgagcttgtggagaccaggccagcaggagatggaaccttccagaagtgggcagctgtggtggtgccttctggagaagagcagagatacacgtgccatgtgcagcacgaggggctgccagagcccctcaccctgagatgggagcca

SEQ ID NO: 107 - DNA, 828 bp, synthetic construct
  misc_feature 1..828: note = Synthetic: HLA-C*07:02
  source 1..828: mol_type = other DNA
tgctcccactccatgaggtatttcgacaccgccgtgtcccggcccggccgcggagagccccgcttcatctcagtgggctacgtggacgacacgcagttcgtgcggttcgacagcgacgccgcgagtccgagaggggagccgcgggcgccgtgggtggagcaggaggggccggagtattgggaccgggagacacagaagtacaagcgccaggcacaggctgaccgagtgagcctgcggaacctgcgcggctgctacaaccagagcgaggacgggtctcacaccctccagaggatgtctggctgcgacctggggcccgacgggcgcctcctccgcgggtatgaccagtccgcctacgacggcaaggattacatcgccctgaacgaggacctgcgctcctggaccgccgcggacaccgcggctcagatcacccagcgcaagttggaggcggcccgtgcggcggagcagctgagagcctacctggagggcacgtgcgtggagtggctccgcagatacctggagaacgggaaggagacgctgcagcgcgcagaacccccaaagacacacgtgacccaccaccccctctctgaccatgaggccaccctgaggtgctgggccctgggcttctaccctgcggagatcacactgacctggcagcgggatggggaggaccagacccaggacaccgagcttgtggagaccaggccagcaggagatggaaccttccagaagtgggcagctgtggtggtgccttctggacaagagcagagatacacgtgccatatgcagcacgaggggctgcaagagcccctcaccctgagctgggagcca

SEQ ID NO: 108 - DNA, 828 bp, synthetic construct
  misc_feature 1..828: note = Synthetic: HLA-C*16:01
  source 1..828: mol_type = other DNA
tgctcccactccatgaggtatttctacaccgccgtgtcccggcccggccgcggagagccccgcttcatcgcagtgggctacgtggacgacacgcagttcgtgcggttcgacagcgacgccgcgagtccaagaggggagccgcgggcgccgtgggtggagcaggaggggccggagtattgggaccgggagacacagaagtacaagcgccaggcacagactgaccgagtgagcctgcggaacctgcgcggctgctacaaccagagcgaggccgggtctcacaccctccagtggatgtatggctgcgacctggggcccgacgggcgcctcctccgcgggtatgaccagtccgcctacgacggcaaggattacatcgccctgaacgaggacctgcgctcctggaccgccgcggacacggcggctcagatcacccagcgcaagtgggaggcggcccgtgcggcggagcagcagagagcctacctggagggcacgtgcgtggagtggctccgcagatacctggagaacgggaaggagacgctgcagcgcgcggaacacccaaagacacacgtgacccaccatctcgtctctgaccatgaggccaccctgaggtgctgggccctgggcttctaccctgcggagatcacactgacctggcagcgggatggcgaggaccaaactcaggacaccgagcttgtggagaccaggccagcaggagatggaaccttccagaagtgggcagctgtggtggtgccttctggagaagagcagagatacacgtgccatgtgcagcacgaggggctgccggagcccctcaccctgagatgggagcca

SEQ ID NO: 109 - DNA, 828 bp, synthetic construct
  misc_feature 1..828: note = Synthetic: HLA-A*25:01
  source 1..828: mol_type = other DNA
ggctcccactccatgaggtatttctacacctccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccggaacacacggaatgtgaaggcccactcacagactgaccgagagagcctgcggatcgcgctccgctgctacaaccagagcgaggacggttctcacaccatccagaggatgtatggctgcgacgtggggccggacgggcgcttcctccgcgggtaccagcaggacgcttacgacggcaaggattacatcgccctgaacgaggacctgcgctcttggaccgcggcggacatggcggctcagatcacccagcgcaagtgggagacggcccatgaggcggagcagtggagagcctacctggagggccggtgcgtggagtggctccgcagatacctggagaacgggaaggagacgctgcagcgcacggacgcccccaagacgcatatgactcaccacgctgtctctgaccatgaggccaccctgaggtgctgggccctgagcttctaccctgcggagatcacactgacctggcagcgggatggggaggaccagacccaggacacggagctcgtggagaccaggcctgcaggggatgggaccttccagaagtgggcgtctgtggtggtgccttctggacaggagcagagatacacctgccatgtgcagcatgagggtctgcccaagcccctcaccctgagatgggagccg

SEQ ID NO: 110 - DNA, 828 bp, synthetic construct
  misc_feature 1..828: note = Synthetic: HLA-A*26:01
  source 1..828: mol_type = other DNA
ggctcccactccatgaggtatttctacacctccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccggaacacacggaatgtgaaggcccactcacagactgaccgagcgaacctggggaccctgcgcggctgctacaaccagagcgaggacggttctcacaccatccagaggatgtatggctgcgacgtggggccggacgggcgcttcctccgcgggtaccagcaggacgcttacgacggcaaggattacatcgccctgaacgaggacctgcgctcttggaccgcggcggacatggcggctcagatcacccagcgcaagtgggagacggcccatgaggcggagcagtggagagcctacctggagggccggtgcgtggagtggctccgcagatacctggagaacgggaaggagacgctgcagcgcacggacgcccccaagacgcatatgactcaccacgctgtctctgaccatgaggccaccctgaggtgctgggccctgagcttctaccctgcggagatcacactgacctggcagcgggatggggaggaccagacccaggacacggagctcgtggagaccaggcctgcaggggatgggaccttccagaagtgggcgtctgtggtggtgccttctggacaggagcagagatacacctgccatgtgcagcatgagggtctgcccaagcccctcaccctgagatgggagccg

SEQ ID NO: 111 - DNA, 828 bp, synthetic construct
  misc_feature 1..828: note = Synthetic: HLA-A*29:02
  source 1..828: mol_type = other DNA
ggctcccactccatgaggtatttcaccacatccgtgtcccggcccggccgcggggagccccgcttcatcgccgtgggctacgtggacgacacgcagttcgtgcggtttgacagcgacgccgcgagccagaggatggagccgcgggcaccgtggatagagcaggaggggccggagtattgggacctgcagacacggaatgtgaaggcccagtcacagactgaccgagcgaacctggggaccctgcgcggctgctacaaccagagcgaggccggttctcacaccatccagatgatgtatggctgcgacgtggggtcggacgggcgcttcctccgcgggtaccggcaggacgcctacgacggcaaggattacatcgccttgaacgaggacctgcgctcttggaccgcggcggacatggcggctcagatcacccagcgcaagtgggaggcggcccgtgtggcggagcagttgagagcctacctggagggcacgtgcgtggagtggctccgcagatacctggagaacgggaaggagacgctgcagcgcacggacgcccccaagacgcatatgactcaccacgctgtctctgaccatgaggccaccctgaggtgctgggccctgagcttctaccctgcggagatcacactgacctggcagcgggatggggaggaccagacccaggacacggagcttgtggagaccaggcctgcaggggatggaaccttccagaagtgggcgtctgtggtggtgccttctggacaggagcagagatacacctgccatgtgcagcatgagggtctgcccaagcccctcaccctgagatgggagccg

SEQ ID NO: 112 - DNA, 828 bp, synthetic construct
  misc_feature 1..828: note = Synthetic: HLA-A*68:02
  source 1..828: mol_type = other DNA
ggctcccactccatgaggtatttctacacctccatgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccggaacacacggaatgtgaaggcccagtcacagactgaccgagtggacctggggaccctgcgcggctgctacaaccagagcgaggccggttctcacaccatccagaggatgtatggctgcgacgtggggccggacgggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagtggagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggtggctgtggtggtgccttctggacaggagcagagatacacctgccatgtgcagcatgagggtttgcccaagcccctcaccctgagatgggagccg

SEQ ID NO: 113 - DNA, 828 bp, synthetic construct
  misc_feature 1..828: note = Synthetic: HLA-B*13:02
  source 1..828: mol_type = other DNA
ggctcccactccatgaggtatttctacaccgccatgtcccggcccggccgcggggagccccgcttcatcaccgtgggctacgtggacgacacccagttcgtgaggttcgacagcgacgccacgagtccgaggatggcgccccgggcgccatggatagagcaggaggggccggagtattgggaccgggagacacagatctccaagaccaacacacagacttaccgagagaacctgcgcaccgcgctccgctgctacaaccagagcgaggccgggtctcacacttggcagacgatgtatggctgcgacctggggccggacgggcgcctcctccgcgggcataaccagttagcctacgacggcaaggattacatcgccctgaacgaggacctgagctcctggaccgcggcggacaccgcggctcagatcacccagctcaagtgggaggcggcccgtgtggcggagcagctgagagcctacctggagggcgagtgcgtggagtggctccgcagatacctggagaacgggaaggagacgctgcagcgcgcggaccccccaaagacacacgtgacccaccaccccatctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagcca

SEQ ID NO: 114 - DNA, 828 bp, synthetic construct
  misc_feature 1..828: note = Synthetic: HLA-B*15:07
  source 1..828: mol_type = other DNA
ggctcccactccatgaggtatttctacaccgccatgtcccggcccggccgcggggagccccgcttcatcgcagtgggctacgtggacgacacccagttcgtgaggttcgacagcgacgccgcgagtccgaggatggcgccccgggcgccatggatagagcaggaggggccggagtattgggaccgggagacacagatctccaagaccaacacacagacttaccgagagagcctgcggaacctgcgcggctgctacaaccagagcgaggccgggtctcacaccctccagagcatgtacggctgcgacgtggggccggacgggcgcctcctccgcgggcatgaccagtccgcctacgacggcaaggattacatcgccctgaacgaggacctgagctcctggaccgcggcggacacggcggctcagatcacccagcgcaagtgggaggcggcccgtgaggcggagcagtggagagcctacctggagggcctgtgcgtggagtggctccgcagatacctggagaacgggaaggagacgctgcagcgcgcggaccccccaaagacacatgtgacccaccaccccatctctgaccatgaggccaccctgaggtgctgggccctgggcttctaccctgcggagatcacactgacctggcagcgggatggcgaggaccaaactcaggacaccgagcttgtggagaccagaccagcaggagatagaaccttccagaagtgggcagctgtggtggtgccttctggagaagagcagagatacacatgccatgtacagcatgaggggctgccgaagcccctcaccctgagatgggagcca

SEQ ID NO: 115 - DNA, 828 bp, synthetic construct
  misc_feature 1..828: note = Synthetic: HLA-B*27:05
  source 1..828: mol_type = other DNA
ggctcccactccatgaggtatttccacacctccgtgtcccggcccggccgcggggagccccgcttcatcaccgtgggctacgtggacgacacgctgttcgtgaggttcgacagcgacgccgcgagtccgagagaggagccgcgggcgccgtggatagagcaggaggggccggagtattgggaccgggagacacagatctgcaaggccaaggcacagactgaccgagaggacctgcggaccctgctccgctgctacaaccagagcgaggccgggtctcacaccctccagaatatgtatggctgcgacgtggggccggacgggcgcctcctccgcgggtaccaccaggacgcctacgacggcaaggattacatcgccctgaacgaggacctgagctcctggaccgccgcggacacggcggctcagatcacccagcgcaagtgggaggcggcccgtgtggcggagcagctgagagcctacctggagggcgagtgcgtggagtggctccgcagatacctggagaacgggaaggagacgctgcagcgcgcggaccccccaaagacacacgtgacccaccaccccatctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccg

SEQ ID NO: 116 - DNA, 828 bp, synthetic construct
  misc_feature 1..828: note = Synthetic: HLA-B*35:03
  source 1..828: mol_type = other DNA
ggctcccactccatgaggtatttctacaccgccatgtcccggcccggccgcggggagccccgcttcatcgcagtgggctacgtggacgacacccagttcgtgaggttcgacagcgacgccgcgagtccgaggacggagccccgggcgccatggatagagcaggaggggccggagtattgggaccggaacacacagatcttcaagaccaacacacagacttaccgagagagcctgcggaacctgcgcggctgctacaaccagagcgaggccgggtctcacatcatccagaggatgtatggctgcgacctggggcccgacgggcgcctcctccgcgggcatgaccagttcgcctacgacggcaaggattacatcgccctgaacgaggacctgagctcctggaccgcggcggacaccgcggctcagatcacccagcgcaagtgggaggcggcccgtgtggcggagcagctgagagcctacctggagggcctgtgcgtggagtggctccgcagatacctggagaacgggaaggagacgctgcagcgcgcggaccccccaaagacacacgtgacccaccaccccgtctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagcca

SEQ ID NO: 117 - DNA, 828 bp, synthetic construct
  misc_feature 1..828: note = Synthetic: HLA-B*37:01
  source 1..828: mol_type = other DNA
ggctcccactccatgaggtatttccacacctccgtgtcccggcccggccgcggggagccccgcttcatctcagtgggctacgtggacgacacccagttcgtgaggttcgacagcgacgccgcgagtccgaggacggagccccgggcgccgtggatagagcaggaggggccggagtattgggaccgggagacacagatctccaagaccaacacacagacttaccgagaggacctgcggaccctgctccgctgctacaaccagagcgaggccgggtctcacaccatccagaggatgtctggctgcgacgtggggccggacgggcgcctcctccgcgggtataaccagttcgcctacgacggcaaggattacatcgccctgaacgaggacctgagctcctggaccgcggcggacaccgcggctcagatcacccagcgcaagtgggaggcggcccgtgtggcggagcaggacagagcctacctggagggcacgtgcgtggagtggctccgcagatacctggagaacgggaaggagacgctgcagcgcgcggaccccccaaagacacatgtgacccaccaccccatctctgaccatgaggccaccctgaggtgctgggccctgggcttctaccctgcggagatcacactgacctggcagcgggatggcgaggaccaaactcaggacaccgagcttgtggagaccagaccagcaggagatagaaccttccagaagtgggcagctgtggtggtgccttctggagaagagcagagatacacatgccatgtacagcatgaggggctgccgaagcccctcaccctgagatgggagcca

SEQ ID NO: 118 - DNA, 828 bp, synthetic construct
  misc_feature 1..828: note = Synthetic: HLA-B*38:01
  source 1..828: mol_type = other DNA
ggctcccactccatgaggtatttctacacctccgtgtcccggcccggccgcggggagccccgcttcatctcagtgggctacgtggacgacacgcagttcgtgaggttcgacagcgacgccgcgagtccgagagaggagccgcgggcgccgtggatagagcaggaggggccggaatattgggaccggaacacacagatctgcaagaccaacacacagacttaccgagagaacctgcggatcgcgctccgctgctacaaccagagcgaggccgggtctcacaccctccagaggatgtacggctgcgacgtggggccggacgggcgcctcctccgcgggcataaccagttcgcctacgacggcaaggattacatcgccctgaacgaggacctgagctcctggaccgcggcggacaccgcggctcagatcacccagcgcaagtgggaggcggcccgtgtggcggagcagctgagaacctacctggagggcacgtgcgtggagtggctccgcagatacctggagaacgggaaggagacgctgcagcgcgcggaccccccaaagacacatgtgacccaccaccccatctctgaccatgaggccaccctgaggtgctgggccctgggcttctaccctgcggagatcacactgacctggcagcgggatggcgaggaccaaactcaggacaccgagcttgtggagaccagaccagcaggagacagaaccttccagaagtgggcagctgtggtggtgccttctggagaagagcagagatacacatgccatgtacagcatgaggggctgccgaagcccctcaccctgagatgggagcca

SEQ ID NO: 119 - DNA, 828 bp, synthetic construct
  misc_feature 1..828: note = Synthetic: HLA-B*41:02
  source 1..828: mol_type = other DNA
ggctcccactccatgaggtatttccacaccgccatgtcccggcccggccgcggggagccccgcttcatcaccgtgggctacgtggacgacacgctgttcgtgaggttcgacagcgacgccacgagtccgaggaaggagccgcgggcgccatggatagagcaggaggggccggagtattgggaccgggagacacagatctccaagaccaacacacagacttaccgagagagcctgcggaacctgcgcggctgctacaaccagagcgaggccgggtctcacaccctccagagcatgtacggctgcgacgtggggccggacgggcgcctcctccgcgggcataaccagtacgcctacgacggcaaggattacatcgccctgaacgaggacctgcgctcctggaccgcggcggacaccgcggctcagatcacccagcgcaagtgggaggcggcccgtgtggcggagcaggacagagcctacctggagggcacgtgcgtggagtggctccgcagatacctggagaacgggaaggacacgctggagcgcgcggaccccccaaagacacacgtgacccaccaccccatctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccg

SEQ ID NO: 120 - DNA, 828 bp, synthetic construct
  misc_feature 1..828: note = Synthetic: HLA-B*44:05
  source 1..828: mol_type = other DNA
ggctcccactccatgaggtatttctacaccgccatgtcccggcccggccgcggggagccccgcttcatcaccgtgggctacgtggacgacacgctgttcgtgaggttcgacagcgacgccacgagtccgaggaaggagccgcgggcgccatggatagagcaggaggggccggagtattgggaccgggagacacagatctccaagaccaacacacagacttaccgagagaacctgcgcaccgcgctccgctgctacaaccagagcgaggccgggtctcacatcatccagaggatgtacggctgcgacgtggggccggacgggcgcctcctccgcgggtatgaccagtacgcctacgacggcaaggattacatcgccctgaacgaggacctgagctcctggaccgcggcggacaccgcggctcagatcacccagcgcaagtgggaggcggcccgtgtggcggagcaggacagagcctacctggagggcctgtgcgtggagtcgctccgcagatacctggagaacgggaaggagacgctgcagcgcgcggaccccccaaagacacatgtgacccaccaccccatctctgaccatgaggtcaccctgaggtgctgggccctgggcttctaccctgcggagatcacactgacctggcagcgggatggcgaggaccaaactcaggacaccgagcttgtggagaccagaccagcaggagatagaaccttccagaagtgggcagctgtggtggtgccttctggagaagagcagagatacacatgccatgtacagcatgaggggctgccgaagcccctcaccctgagatgggagccg

SEQ ID NO: 121 - DNA, 828 bp, synthetic construct
  misc_feature 1..828: note = Synthetic: HLA-B*49:01
  source 1..828: mol_type = other DNA
ggctcccactccatgaggtatttccacaccgccatgtcccggcccggccgcggggagccccgcttcatcaccgtgggctacgtggacgacacgctgttcgtgaggttcgacagcgacgccacgagtccgaggaaggagccgcgggcgccatggatagagcaggaggggccggagtattgggaccgggagacacagatctccaagaccaacacacagacttaccgagagaacctgcggatcgcgctccgctgctacaaccagagcgaggccgggtctcacacttggcagaggatgtatggctgcgacctggggcccgacgggcgcctcctccgcgggtataaccagttagcctacgacggcaaggattacatcgccctgaacgaggacctgagctcctggaccgcggcggacaccgcggctcagatcacccagcgcaagtgggaggcggcccgtgaggcggagcagctgagagcctacctggagggcctgtgcgtggagtggctccgcagatacctggagaacgggaaggagacgctgcagcgcgcggaccccccaaagacacatgtgacccaccaccccatctctgaccatgaggccaccctgaggtgctgggccctgggcttctaccctgcggagatcacactgacctggcagcgggatggcgaggaccaaactcaggacaccgagcttgtggagaccagaccagcaggagatagaaccttccagaagtgggcagctgtggtggtgccttctggagaagagcagagatacacatgccatgtacagcatgaggggctgccgaagcccctcaccctgagatgggagcca

SEQ ID NO: 122 - DNA, 828 bp, synthetic construct
  misc_feature 1..828: note = Synthetic: HLA-B*52:01
  source 1..828: mol_type = other DNA
ggctcccactccatgaggtatttctacaccgccatgtcccggcccggccgcggggagccccgcttcatcgcagtgggctacgtggacgacacccagttcgtgaggttcgacagcgacgccgcgagtccgaggacggagccccgggcgccatggatagagcaggaggggccggagtattgggaccgggagacacagatctccaagaccaacacacagacttaccgagagaacctgcggatcgcgctccgctgctacaaccagagcgaggccgggtctcacacttggcagacgatgtatggctgcgacgtggggccggacgggcgcctcctccgcgggcataaccagtacgcctacgacggcaaagattacatcgccctgaacgaggacctgagctcctggaccgcggcggacaccgcggctcagatcacccagcgcaagtgggaggcggcccgtgaggcggagcagctgagagcctacctggagggcctgtgcgtggagtggctccgcagacacctggagaacgggaaggagacgctgcagcgcgcggaccccccaaagacacacgtgacccaccaccccgtctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagcca

SEQ ID NO: 123 - DNA, 828 bp, synthetic construct
  misc_feature 1..828: note = Synthetic: HLA-B*55:01
  source 1..828: mol_type = other DNA
ggctcccactccatgaggtatttctacaccgccatgtcccggcccggccgcggggagccccgcttcatcgcagtgggctacgtggacgacacgcagttcgtgaggttcgacagcgacgccgcgagtccgagagaggagccgcgggcgccgtggatagagcaggaggggccggagtattgggaccggaacacacagatctacaaggcccaggcacagactgaccgagagagcctgcggaacctgcgcggctgctacaaccagagcgaggccgggtctcacacttggcagacgatgtatggctgcgacctggggccggacgggcgcctcctccgcgggcataaccagttagcctacgacggcaaggattacatcgccctgaacgaggacctgagctcctggaccgcggcggacaccgcggctcagatcacccagcgcaagtgggaggcggcccgtgaggcggagcagctgagagcctacctggagggcacgtgcgtggagtggctccgcagatacctggagaacgggaaggagacgctgcagcgcgcggaccccccaaagacacacgtgacccaccaccccatctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagcca

SEQ ID NO: 124 - DNA, 828 bp, synthetic construct
  misc_feature 1..828: note = Synthetic: HLA-C*03:03
  source 1..828: mol_type = other DNA
ggctcccactccatgaggtatttctacaccgctgtgtcccggcccggccgcggggagccccacttcatcgcagtgggctacgtggacgacacgcagttcgtgcggttcgacagcgacgccgcgagtccgagaggggagccgcgggcgccgtgggtggagcaggaggggccggagtattgggaccgggagacacagaagtacaagcgccaggcacagactgaccgagtgagcctgcggaacctgcgcggctgctacaaccagagcgaggccaggtctcacatcatccagaggatgtatggctgcgacgtggggcccgacgggcgcctcctccgcgggtatgaccagtacgcctacgacggcaaggattacatcgccctgaacgaggatctgcgctcctggaccgccgcggacacggcggctcagatcacccagcgcaagtgggaggcggcccgtgaggcggagcagctgagagcctacctggagggcctgtgcgtggagtggctccgcagatacctgaagaatgggaaggagacgctgcagcgcgcggaacacccaaagacacacgtgacccaccatcccgtctctgaccatgaggccaccctgaggtgctgggccctgggcttctaccctgcggagatcacactgacctggcagtgggatggggaggaccaaactcaggacactgagcttgtggagaccaggccagcaggagatggaaccttccagaagtgggcagctgtggtggtgccttctggagaagagcagagatacacgtgccatgtgcagcacgaggggctgccggagcccctcaccctgagatgggagccg

SEQ ID NO: 125 - DNA, 828 bp, synthetic construct
  misc_feature 1..828: note = Synthetic: HLA-C*07:04
  source 1..828: mol_type = other DNA
tgctcccactccatgaggtatttcgacaccgccgtgtcccggcccggccgcggagagccccgcttcatctcagtgggctacgtggacgacacgcagttcgtgcggttcgacagcgacgccgcgagtccgagaggggagccccgggcgccgtgggtggagcaggaggggccggagtattgggaccgggagacacagaagtacaagcgccaggcacaggctgaccgagtgagcctgcggaacctgcgcggctgctacaaccagagcgaggacgggtctcacaccttccagaggatgtatggctgcgacctggggcccgacgggcgcctcctccgcgggtatgaccagttcgcctacgacggcaaggattacatcgccctgaacgaggacctgcgctcctggaccgccgcggacaccgcggctcagatcacccagcgcaagttggaggcggcccgtgcggcggagcaggacagagcctacctggagggcacgtgcgtggagtggctccgcagatacctggagaacgggaagaagacgctgcagcgcgcggaacccccaaagacacacgtgacccaccaccccctctctgaccatgaggccaccctgaggtgctgggccctgggcttctaccctgcggagatcacactgacctggcagcgggatggggaggaccagacccaggacaccgagcttgtggagaccaggccagcaggagatggaaccttccagaagtgggcagctgtggtggtgccttctggacaagagcagagatacacgtgccatatgcagcacgaggggctgcaagagcccctcaccctgagctgggagcca

SEQ ID NO: 126 - DNA, 828 bp, synthetic construct
  misc_feature 1..828: note = Synthetic: HLA-C*08:01
  source 1..828: mol_type = other DNA
tgctcccactccatgaggtatttctacaccgccgtgtcccggcccggccgcggagagccccgcttcatcgcagtgggctacgtggacgacacgcagttcgtgcagttcgacagcgacgccgcgagtccaagaggggagccgcgggcgccgtgggtggagcaggaggggccggagtattgggaccgggagacacagaagtacaagcgccaggcacagactgaccgagtgagcctgcggaacctgcgcggctgctacaaccagagcgaggccgggtctcacaccctccagaggatgtatggctgcgacctggggcccgacgggcgcctcctccgcgggtataaccagttcgcctacgacggcaaggattacatcgccctgaatgaggacctgcgctcctggaccgccgcggacacggcggctcagatcacccagcgcaagtgggaggcggcccgtacggcggagcagctgagagcctacctggagggcacgtgcgtggagtggctccgcagatacctggagaacgggaagaagacgctgcagcgcgcggaacacccaaagacacacgtgacccaccatcccgtctctgaccatgaggccaccctgaggtgctgggccctgggcttctaccctgcggagatcacactgacctggcagcgggatggcgaggaccaaactcaggacaccgagcttgtggagaccaggccagcaggagatggaaccttccagaagtgggcagctgtggtggtgccttctggagaagagcagagatacacgtgccatgtgcagcacgaggggctgccagagcccctcaccctgagatgggggcca

SEQ ID NO: 127 - DNA, 828 bp, synthetic construct
  misc_feature 1..828: note = Synthetic: HLA-C*08:02
  source 1..828: mol_type = other DNA
tgctcccactccatgaggtatttctacaccgccgtgtcccggcccggccgcggagagccccgcttcatcgcagtgggctacgtggacgacacgcagttcgtgcagttcgacagcgacgccgcgagtccaagaggggagccgcgggcgccgtgggtggagcaggaggggccggagtattgggaccgggagacacagaagtacaagcgccaggcacagactgaccgagtgagcctgcggaacctgcgcggctgctacaaccagagcgaggccgggtctcacaccctccagaggatgtatggctgcgacctggggcccgacgggcgcctcctccgcgggtataaccagttcgcctacgacggcaaggattacatcgccctgaatgaggacctgcgctcctggaccgccgcggacaaggcggctcagatcacccagcgcaagtgggaggcggcccgtgaggcggagcagcggagagcctacctggagggcacgtgcgtggagtggctccgcagatacctggagaacgggaagaagacgctgcagcgcgcggaacacccaaagacacacgtgacccaccatcccgtctctgaccatgaggccaccctgaggtgctgggccctgggcttctaccctgcggagatcacactgacctggcagcgggatggcgaggaccaaactcaggacaccgagcttgtggagaccaggccagcaggagatggaaccttccagaagtgggcagctgtggtggtgccttctggagaagagcagagatacacgtgccatgtgcagcacgaggggctgccagagcccctcaccctgagatgggggcca

SEQ ID NO: 128 - DNA, 828 bp, synthetic construct
  misc_feature 1..828: note = Synthetic: HLA-C*12:02
  source 1..828: mol_type = other DNA
tgctcccactccatgaggtatttctacaccgccgtgtcccggcccggccgcggagagccccgcttcatcgcagtgggctacgtggacgacacgcagttcgtgcggttcgacagcgacgccgcgagtccaagaggggagccgcgggcgccgtgggtggagcaggaggggccggagtattgggaccgggagacacagaagtacaagcgccaggcacaggctgaccgagtgagcctgcggaacctgcgcggctgctacaaccagagcgaggccgggtctcacaccctccagaggatgtacggctgcgacctggggcccgacgggcgcctcctccgcgggtatgaccagtccgcctacgacggcaaggattacatcgccctgaacgaggacctgcgctcctggaccgctgcggacacggcggctcagatcacccagcgcaagtgggaggcggcccgtgaggcggagcagtggagagcctacctggagggcacgtgcgtggagtggctccgcagatacctggagaacgggaaggagacgctgcagcgcgcggaacacccaaagacacacgtgacccaccatcccgtctctgaccatgaggccaccctgaggtgctgggccctgggcttctaccctgcggagatcacactgacctggcagcgggatggcgaggaccaaactcaggacaccgagcttgtggagaccaggccagcaggagatggaaccttccagaagtgggcagctgtggtggtgccttctggagaagagcagagatacacgtgccatgtgcagcacgaggggctgccggagcccctcaccctgagatgggagcca

SEQ ID NO: 129 - DNA, 828 bp, synthetic construct
  misc_feature 1..828: note = Synthetic: HLA-C*12:03
  source 1..828: mol_type = other DNA
tgctcccactccatgaggtatttctacaccgccgtgtcccggcccggccgcggagagccccgcttcatcgcagtgggctacgtggacgacacgcagttcgtgcggttcgacagcgacgccgcgagtccaagaggggagccgcgggcgccgtgggtggagcaggaggggccggagtattgggaccgggagacacagaagtacaagcgccaggcacaggctgaccgagtgagcctgcggaacctgcgcggctgctacaaccagagcgaggccgggtctcacaccctccagtggatgtatggctgcgacctggggcccgacgggcgcctcctccgcgggtatgaccagtccgcctacgacggcaaggattacatcgccctgaacgaggacctgcgctcctggactgccgcggacacggcggctcagatcacccagcgcaagtgggaggcggcccgtgaggcggagcagtggagagcctacctggagggcacgtgcgtggagtggctccgcagatacctggagaacgggaaggagacgctgcagcgcgcggaacacccaaagacacacgtgacccaccatcccgtctctgaccatgaggccaccctgaggtgctgggccctgggcttctaccctgcggagatcacactgacctggcagcgggatggcgaggaccaaactcaggacaccgagcttgtggagaccaggccagcaggagatggaaccttccagaagtgggcagctgtggtggtgccttctggagaagagcagagatacacgtgccatgtgcagcacgaggggctgccagagcccctcaccctgagatgggagcca

SEQ ID NO: 130 - DNA, 828 bp, synthetic construct
  misc_feature 1..828: note = Synthetic: HLA-C*14:02
  source 1..828: mol_type = other DNA
tgctcccactccatgaggtatttctccacatccgtgtcccggcccggccgcggggagccccgcttcatcgcagtgggctacgtggacgacacgcagttcgtgcggttcgacagcgacgccgcgagtccgagaggggagccgcgggcgccgtgggtggagcaggaggggccggagtattgggaccgggagacacagaagtacaagcgccaggcacagactgaccgagtgagcctgcggaacctgcgcggctgctacaaccagagcgaggccgggtctcacaccctccagtggatgtttggctgcgacctggggcccgacgggcgcctcctccgcgggtatgaccagtccgcctacgacggcaaggattacatcgccctgaacgaggatctgcgctcctggaccgccgcggacacggcggctcagatcacccagcgcaagtgggaggcggcccgtgaggcggagcagcggagagcctacctggagggcacgtgcgtggagtggctccgcagatacctggagaacgggaaggagacgctgcagcgcgcggaacacccaaagacacacgtgacccaccatcccgtctctgaccatgaggccaccctgaggtgctgggccctgggcttctaccctgcggagatcacactgacctggcagtgggatggggaggaccaaactcaggacaccgagcttgtggagaccaggccagcaggagatggaaccttccagaagtgggcagctgtggtggtgccttctggagaagagcagagatacacgtgccatgtgcagcacgaggggctgccggagcccctcaccctgagatgggagccg

SEQ ID NO: 131 - DNA, 828 bp, synthetic construct
  misc_feature 1..828: note = Synthetic: HLA-C*15:02
  source 1..828: mol_type = other DNA
tgctcccactccatgaggtatttctacaccgctgtgtcccggcccggccgcggagagccccacttcatcgcagtgggctacgtggacgacacgcagttcgtgcggttcgacagcgacgccgcgagtccaagaggggagccgcgggcgccgtgggtggagcaggaggggccggagtattgggaccgggagacacagaactacaagcgccaggcacagactgaccgagtgaacctgcggaaactgcgcggctgctacaaccagagcgaggccgggtctcacatcatccagaggatgtatggctgcgacctggggcccgacgggcgcctcctccgcgggcatgaccagttagcctacgacggcaaggattacatcgccctgaacgaggacctgcgctcctggaccgccgcggacacggcggctcagatcacccagcgcaagtgggaggcggcccgtgaggcggagcagctgagagcctacctggagggcacgtgcgtggagtggctccgcagatacctggagaacgggaaggagacgctgcagcgcgcggaacacccaaagacacacgtgacccaccatcccgtctctgaccatgaggccaccctgaggtgctgggccctgggcttctaccctgcggagatcacactgacctggcagcgggatggcgaggaccaaactcaggacaccgagcttgtggagaccaggccagcaggagatggaaccttccagaagtgggcagctgtggtggtgccttctggagaagagcagagatacacgtgccatgtgcagcacgaggggctgccggagcccctcaccctgagatgggagcca

SEQ ID NO: 132 - DNA, 828 bp, synthetic construct
  misc_feature 1..828: note = Synthetic: HLA-C*17:01
  source 1..828: mol_type = other DNA
ggctcccactccatgaggtatttctacaccgccgtgtcccggcccggccgcggagagccccgcttcatcgcagtgggctacgtggacgacacgcagttcgtgcggttcgacagcgacgccgcgagtccgagaggggagccgcgggcgccgtgggtggagcaggaggggccggagtattgggaccgggagacacagaagtacaagcgccaggcacaggctgaccgagtgaacctgcggaaactgcgcggctgctacaaccagagcgaggccggttctcacaccatccagaggatgtatggctgcgacctggggcccgacgggcgcctcctccgcgggtataaccagttcgcctacgacggcaaggattacatcgccctgaacgaggacctgcgctcctggaccgcggcggacacggcggctcagatctcccagcgcaagttggaggcggcccgtgaggcggagcagctgagagcctacctggagggcgagtgcgtggagtggctccgcggatacctggagaacgggaaggagacgctgcagcgcgcggaacgcccaaagacacacgtgacccaccatcccgtctctgaccatgaggccaccctgaggtgctgggccctgggcttctaccctgcggagatcacactgacctggcagcgggatggggaggaccaaactcaggacaccgagcttgtggagaccaggccagcaggagatggaaccttccagaagtgggcagctgtggtggtgccttctggacaagaacagagatacacgtgccatgtgcagcacgaggggctgcaggagccctgcaccctgagatggaagccg

SEQ ID NO: 133 - protein, 15 aa, synthetic construct
  REGION 1..15: note = Synthetic: Linker A
GAGASGGGGSGGGGS

SEQ ID NO: 134 - protein, 15 aa, synthetic construct
  REGION 1..15: note = Synthetic: Linker B
GGGASGGGGSGGGGS

SEQ ID NO: 135 - protein, 98 aa, synthetic construct
  REGION 1..98: note = Synthetic: HPV16-E7
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 136 - protein, 158 aa, synthetic construct
  REGION 1..158: note = Synthetic: HPV16-E6
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 137 - protein, 276 aa, synthetic construct
  REGION 1..276: note = Synthetic: HLA-A*01:01
GSHSMRYFFTSVSRPGRGEPRFIAVGYVDDTQFVRFDSDAASQKMEPRAPWIEQEGPEYWDQETRNMKAHSQTDRANLGTLRGCYNQSEDGSHTIQIMYGCDVGPDGRFLRGYRQDAYDGKDYIALNEDLRSWTAADMCAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 138 - protein, 276 aa, synthetic construct
  REGION 1..276: note = Synthetic: HLA-A*02:01
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEP

SEQ ID NO: 139 - protein, 276 aa, synthetic construct
  REGION 1..276: note = Synthetic: HLA-A*03:01
GSHSMRYFFTSVSRPGRGEPRFIAVGYVDDTQFVRFDSDAASQRMEPRAPWIEQEGPEYWDQETRNVKAQSQTDRVDLGTLRGCYNQSEAGSHTIQIMYGCDVGSDGRFLRGYRQDAYDGKDYIALNEDLRSWTAADMCAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 140 - protein, 276 aa, synthetic construct
  REGION 1..276: note = Synthetic: HLA-A*24:02
GSHSMRYFSTSVSRPGRGEPRFIAVGYVDDTQFVRFDSDAASQRMEPRAPWIEQEGPEYWDEETGKVKAHSQTDRENLRIALRCYNQSEAGSHTLQMMFGCDVGSDGRFLRGYHQYAYDGKDYIALKEDLRSWTAADMCAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 141 - protein, 276 aa, synthetic construct
  REGION 1..276: note = Synthetic: HLA-A*30:02
GSHSMRYFSTSVSRPGSGEPRFIAVGYVDDTQFVRFDSDAASQRMEPRAPWIEQERPEYWDQETRNVKAHSQTDRENLGTLRGCYNQSEAGSHTIQIMYGCDVGSDGRFLRGYEQHAYDGKDYIALNEDLRSWTAADMCAQITQRKWEAARRAEQLRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 142 - protein, 276 aa, synthetic construct
  REGION 1..276: note = Synthetic: HLA-A*31:01
GSHSMRYFTTSVSRPGRGEPRFIAVGYVDDTQFVRFDSDAASQRMEPRAPWIEQERPEYWDQETRNVKAHSQIDRVDLGTLRGCYNQSEAGSHTIQMMYGCDVGSDGRFLRGYQQDAYDGKDYIALNEDLRSWTAADMCAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 143 - protein, 276 aa, synthetic construct
  REGION 1..276: note = Synthetic: HLA-A*32:01
GSHSMRYFFTSVSRPGRGEPRFIAVGYVDDTQFVRFDSDAASQRMEPRAPWIEQEGPEYWDQETRNVKAHSQTDRESLRIALRCYNQSEAGSHTIQMMYGCDVGPDGRLLRGYQQDAYDGKDYIALNEDLRSWTAADMCAQITQRKWEAARVAEQLRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 144 - protein, 276 aa, synthetic construct
  REGION 1..276: note = Synthetic: HLA-A*33:01
GSHSMRYFTTSVSRPGRGEPRFIAVGYVDDTQFVRFDSDAASQRMEPRAPWIEQEGPEYWDRNTRNVKAHSQIDRVDLGTLRGCYNQSEAGSHTIQMMYGCDVGSDGRFLRGYQQDAYDGKDYIALNEDLRSWTAADMCAQITQRKWEAARVAEQLRAYLEGTCVEWLRRHLENGKETLQRTDPPRTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 145 - protein, 276 aa, synthetic construct
  REGION 1..276: note = Synthetic: HLA-A*68:01
GSHSMRYFYTSVSRPGRGEPRFIAVGYVDDTQFVRFDSDAASQRMEPRAPWIEQEGPEYWDRNTRNVKAQSQTDRVDLGTLRGCYNQSEAGSHTIQMMYGCDVGSDGRFLRGYRQDAYDGKDYIALKEDLRSWTAADMCAQTTKHKWEAAHVAEQWRAYLEGTCVEWLRRYLENGKETLQRTDAPKTHMTHHAVSDHEATLRCWALSFYPAEITLTWQRDGEDQTQDTELVETRPAGDGTFQKWVAVVVPSGQEQRYTCHVQHEGLPKPLTLRWEP

SEQ ID NO: 146 - protein, 276 aa, synthetic construct
  REGION 1..276: note = Synthetic: HLA-B*07:02
GSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHDQYAYDGKDYIALNEDLRSWTAADTCAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 147 - protein, 276 aa, synthetic construct
  REGION 1..276: note = Synthetic: HLA-B*14:02
GSHSMRYFYTAVSRPGRGEPRFISVGYVDDTQFVRFDSDAASPREEPRAPWIEQEGPEYWDRNTQICKTNTQTDRESLRNLRGCYNQSEAGSHTLQWMYGCDVGPDGRLLRGYNQFAYDGKDYIALNEDLSSWTAADTCAQITQRKWEAAREAEQLRAYLEGTCVEWLRRHLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 148 - protein, 276 aa, synthetic construct
  REGION 1..276: note = Synthetic: HLA-B*18:01
GSHSMRYFHTSVSRPGRGEPRFISVGYVDGTQFVRFDSDAASPRTEPRAPWIEQEGPEYWDRNTQISKTNTQTYRESLRNLRGCYNQSEAGSHTLQRMYGCDVGPDGRLLRGHDQSAYDGKDYIALNEDLSSWTAADTCAQITQRKWEAARVAEQLRAYLEGTCVEWLRRHLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 149 - protein, 276 aa, synthetic construct
  REGION 1..276: note = Synthetic: HLA-B*27:02
GSHSMRYFHTSVSRPGRGEPRFITVGYVDDTLFVRFDSDAASPREEPRAPWIEQEGPEYWDRETQICKAKAQTDRENLRIALRCYNQSEAGSHTLQNMYGCDVGPDGRLLRGYHQDAYDGKDYIALNEDLSSWTAADTCAQITQRKWEAARVAEQLRAYLEGECVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 150 - protein, 276 aa, synthetic construct
  REGION 1..276: note = Synthetic: HLA-B*39:01
GSHSMRYFYTSVSRPGRGEPRFISVGYVDDTQFVRFDSDAASPREEPRAPWIEQEGPEYWDRNTQICKTNTQTDRESLRNLRGCYNQSEAGSHTLQRMYGCDVGPDGRLLRGHNQFAYDGKDYIALNEDLSSWTAADTCAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 151 - protein, 276 aa, synthetic construct
  REGION 1..276: note = Synthetic: HLA-B*40:01
GSHSMRYFHTAMSRPGRGEPRFITVGYVDDTLFVRFDSDATSPRKEPRAPWIEQEGPEYWDRETQISKTNTQTYRESLRNLRGCYNQSEAGSHTLQRMYGCDVGPDGRLLRGHNQYAYDGKDYIALNEDLRSWTAADTCAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 152 - protein, 276 aa, synthetic construct
  REGION 1..276: note = Synthetic: HLA-B*44:02
GSHSMRYFYTAMSRPGRGEPRFITVGYVDDTLFVRFDSDATSPRKEPRAPWIEQEGPEYWDRETQISKTNTQTYRENLRTALRCYNQSEAGSHIIQRMYGCDVGPDGRLLRGYDQDAYDGKDYIALNEDLSSWTAADTCAQITQRKWEAARVAEQDRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEP

SEQ ID NO: 153 - protein, 276 aa, synthetic construct
  REGION 1..276: note = Synthetic: HLA-B*46:01
GSHSMRYFYTAMSRPGRGEPRFIAVGYVDDTQFVRFDSDAASPRMAPRAPWIEQEGPEYWDRETQKYKRQAQTDRVSLRNLRGCYNQSEAGSHTLQRMYGCDVGPDGRLLRGHDQSAYDGKDYIALNEDLSSWTAADTCAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 154 - protein, 276 aa, synthetic construct
  REGION 1..276: note = Synthetic: HLA-B*50:01
GSHSMRYFHTAMSRPGRGEPRFITVGYVDDTLFVRFDSDATSPRKEPRAPWIEQEGPEYWDRETQISKTNTQTYRESLRNLRGCYNQSEAGSHTWQRMYGCDLGPDGRLLRGYNQLAYDGKDYIALNEDLSSWTAADTCAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 155 - protein, 276 aa, synthetic construct
  REGION 1..276: note = Synthetic: HLA-B*57:01
GSHSMRYFYTAMSRPGRGEPRFIAVGYVDDTQFVRFDSDAASPRMAPRAPWIEQEGPEYWDGETRNMKASAQTYRENLRIALRCYNQSEAGSHIIQVMYGCDVGPDGRLLRGHDQSAYDGKDYIALNEDLSSWTAADTCAQITQRKWEAARVAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 156 - protein, 276 aa, synthetic construct
  REGION 1..276: note = Synthetic: HLA-B*58:01
GSHSMRYFYTAMSRPGRGEPRFIAVGYVDDTQFVRFDSDAASPRTEPRAPWIEQEGPEYWDGETRNMKASAQTYRENLRIALRCYNQSEAGSHIIQRMYGCDLGPDGRLLRGHDQSAYDGKDYIALNEDLSSWTAADTCAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 157 - protein, 276 aa, synthetic construct
  REGION 1..276: note = Synthetic: HLA-C*02:02
CSHSMRYFYTAVSRPSRGEPHFIAVGYVDDTQFVRFDSDAASPRGEPRAPWVEQEGPEYWDRETQKYKRQAQTDRVNLRKLRGCYNQSEAGSHTLQRMYGCDLGPDGRLLRGYDQSAYDGKDYIALNEDLRSWTAADTCAQITQRKWEAAREAEQWRAYLEGECVEWLRRYLENGKETLQRAEHPKTHVTHHPVSDHEATLRCWALGFYPTEITLTWQRDGEDQTQDTELVETRPAGDGTFQKWAAVVVPSGEEQRYTCHVQHEGLPEPLTLRWEP

SEQ ID NO: 158 - protein, 276 aa, synthetic construct
  REGION 1..276: note = Synthetic: HLA-C*03:04
GSHSMRYFYTAVSRPGRGEPHFIAVGYVDDTQFVRFDSDAASPRGEPRAPWVEQEGPEYWDRETQKYKRQAQTDRVSLRNLRGCYNQSEAGSHIIQRMYGCDVGPDGRLLRGYDQYAYDGKDYIALNEDLRSWTAADTCAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 159 - protein, 276 aa, synthetic construct
  REGION 1..276: note = Synthetic: HLA-C*05:01
CSHSMRYFYTAVSRPGRGEPRFIAVGYVDDTQFVQFDSDAASPRGEPRAPWVEQEGPEYWDRETQKYKRQAQTDRVNLRKLRGCYNQSEAGSHTLQRMYGCDLGPDGRLLRGYNQFAYDGKDYIALNEDLRSWTAADKCAQITQRKWEAAREAEQRRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGP

SEQ ID NO: 160 - protein, 276 aa, synthetic construct
  REGION 1..276: note = Synthetic: HLA-C*07:01
CSHSMRYFDTAVSRPGRGEPRFISVGYVDDTQFVRFDSDAASPRGEPRAPWVEQEGPEYWDRETQNYKRQAQADRVSLRNLRGCYNQSEDGSHTLQRMYGCDLGPDGRLLRGYDQSAYDGKDYIALNEDLRSWTAADTCAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 161 - protein, 276 aa, synthetic construct
  REGION 1..276: note = Synthetic: HLA-A*11:01
GSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMCAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 162 - protein, 276 aa, synthetic construct
  REGION 1..276: note = Synthetic: HLA-A*23:01
GSHSMRYFSTSVSRPGRGEPRFIAVGYVDDTQFVRFDSDAASQRMEPRAPWIEQEGPEYWDEETGKVKAHSQTDRENLRIALRCYNQSEAGSHTLQMMFGCDVGSDGRFLRGYHQYAYDGKDYIALKEDLRSWTAADMCAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 163 - protein, 276 aa, synthetic construct
  REGION 1..276: note = Synthetic: HLA-A*30:01
GSHSMRYFSTSVSRPGSGEPRFIAVGYVDDTQFVRFDSDAASQRMEPRAPWIEQERPEYWDQETRNVKAQSQTDRVDLGTLRGCYNQSEAGSHTIQIMYGCDVGSDGRFLRGYEQHAYDGKDYIALNEDLRSWTAADMCAQITQRKWEAARWAEQLRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 164 - protein, 276 aa, synthetic construct
  REGION 1..276: note = Synthetic: HLA-A*33:03
GSHSMRYFTTSVSRPGRGEPRFIAVGYVDDTQFVRFDSDAASQRMEPRAPWIEQEGPEYWDRNTRNVKAHSQIDRVDLGTLRGCYNQSEAGSHTIQMMYGCDVGSDGRFLRGYQQDAYDGKDYIALNEDLRSWTAADMCAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 165 - protein, 276 aa, synthetic construct
  REGION 1..276: note = Synthetic: HLA-B*08:01
GSHSMRYFDTAMSRPGRGEPRFISVGYVDDTQFVRFDSDAASPREEPRAPWIEQEGPEYWDRNTQIFKTNTQTDRESLRNLRGCYNQSEAGSHTLQSMYGCDVGPDGRLLRGHNQYAYDGKDYIALNEDLRSWTAADTCAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 166 - protein, 276 aa, synthetic construct
  REGION 1..276: note = Synthetic: HLA-B*15:01
GSHSMRYFYTAMSRPGRGEPRFIAVGYVDDTQFVRFDSDAASPRMAPRAPWIEQEGPEYWDRETQISKTNTQTYRESLRNLRGCYNQSEAGSHTLQRMYGCDVGPDGRLLRGHDQSAYDGKDYIALNEDLSSWTAADTCAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 167 - protein, 276 aa, synthetic construct
  REGION 1..276: note = Synthetic: HLA-B*15:03
GSHSMRYFYTAMSRPGRGEPRFISVGYVDDTQFVRFDSDAASPREEPRAPWIEQEGPEYWDRETQISKTNTQTYRESLRNLRGCYNQSEAGSHTLQRMYGCDVGPDGRLLRGHDQSAYDGKDYIALNEDLSSWTAADTCAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 168 - protein, 276 aa, synthetic construct
  REGION 1..276: note = Synthetic: HLA-B*35:01
GSHSMRYFYTAMSRPGRGEPRFIAVGYVDDTQFVRFDSDAASPRTEPRAPWIEQEGPEYWDRNTQIFKTNTQTYRESLRNLRGCYNQSEAGSHIIQRMYGCDLGPDGRLLRGHDQSAYDGKDYIALNEDLSSWTAADTCAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 169 - protein, 276 aa, synthetic construct
  REGION 1..276: note = Synthetic: HLA-B*40:02
GSHSMRYFHTSVSRPGRGEPRFITVGYVDDTLFVRFDSDATSPRKEPRAPWIEQEGPEYWDRETQISKTNTQTYRESLRNLRGCYNQSEAGSHTLQSMYGCDVGPDGRLLRGHNQYAYDGKDYIALNEDLRSWTAADTCAQITQRKWEAARVAEQLRAYLEGECVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 170 - protein, 276 aa, synthetic construct
  REGION 1..276: note = Synthetic: HLA-B*42:01
GSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHNQYAYDGKDYIALNEDLRSWTAADTCAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 171 - protein, 276 aa, synthetic construct
  REGION 1..276: note = Synthetic: HLA-B*44:03
GSHSMRYFYTAMSRPGRGEPRFITVGYVDDTLFVRFDSDATSPRKEPRAPWIEQEGPEYWDRETQISKTNTQTYRENLRTALRCYNQSEAGSHIIQRMYGCDVGPDGRLLRGYDQDAYDGKDYIALNEDLSSWTAADTCAQITQRKWEAARVAEQLRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEP

SEQ ID NO: 172 - protein, 276 aa, synthetic construct
  REGION 1..276: note = Synthetic: HLA-B*51:01
GSHSMRYFYTAMSRPGRGEPRFIAVGYVDDTQFVRFDSDAASPRTEPRAPWIEQEGPEYWDRNTQIFKTNTQTYRENLRIALRCYNQSEAGSHTWQTMYGCDVGPDGRLLRGHNQYAYDGKDYIALNEDLSSWTAADTCAQITQRKWEAAREAEQLRAYLEGLCVEWLRRHLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 173 - protein, 276 aa, synthetic construct
  REGION 1..276: note = Synthetic: HLA-B*53:01
GSHSMRYFYTAMSRPGRGEPRFIAVGYVDDTQFVRFDSDAASPRTEPRAPWIEQEGPEYWDRNTQIFKTNTQTYRENLRIALRCYNQSEAGSHIIQRMYGCDLGPDGRLLRGHDQSAYDGKDYIALNEDLSSWTAADTCAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 174 - protein, 276 aa, synthetic construct
  REGION 1..276: note = Synthetic: HLA-C*01:02
CSHSMKYFFTSVSRPGRGEPRFISVGYVDDTQFVRFDSDAASPRGEPRAPWVEQEGPEYWDRETQKYKRQAQTDRVSLRNLRGCYNQSEAGSHTLQWMCGCDLGPDGRLLRGYDQYAYDGKDYIALNEDLRSWTAADTCAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEP

SEQ ID NO: 175 - protein, 276 aa, synthetic construct
  REGION 1..276: note = Synthetic: HLA-C*04:01
GSHSMRYFSTSVSWPGRGEPRFIAVGYVDDTQFVRFDSDAASPRGEPREPWVEQEGPEYWDRETQKYKRQAQADRVNLRKLRGCYNQSEDGSHTLQRMFGCDLGPDGRLLRGYNQFAYDGKDYIALNEDLRSWTAADTCAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWKP

SEQ ID NO: 176 - protein, 275 aa, synthetic construct
  REGION 1..275: note = Synthetic: HLA-C*06:02
CSHSMRYFDTAVSRPGRGEPRFISVGYVDDTQFVRFDSDAASPRGEPRAPWVEQEGPEYWDRETQKYKRQAQADRVNLRKLRGCYNQSEDGSHTLQWMYGCDLGPDGRLLRGYDQSAYDGKDYIALNEDLRSWTAADTCAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWE

SEQ ID NO: 177 - protein, 276 aa, synthetic construct
  REGION 1..276: note = Synthetic: HLA-C*07:02
CSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTCAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 178 - protein, 276 aa, synthetic construct
  REGION 1..276: note = Synthetic: HLA-C*16:01
CSHSMRYFYTAVSRPGRGEPRFIAVGYVDDTQFVRFDSDAASPRGEPRAPWVEQEGPEYWDRETQKYKRQAQTDRVSLRNLRGCYNQSEAGSHTLQWMYGCDLGPDGRLLRGYDQSAYDGKDYIALNEDLRSWTAADTCAQITQRKWEAARAAEQQRAYLEGTCVEWLRRYLENGKETLQRAEHPKTHVTHHLVSDHEATLRCWALGFYPAEITLTWQRDGEDQTQDTELVETRPAGDGTFQKWAAVVVPSGEEQRYTCHVQHEGLPEPLTLRWEP

SEQ ID NO: 179 - protein, 276 aa, synthetic construct
  REGION 1..276: note = Synthetic: HLA-A*25:01
GSHSMRYFYTSVSRPGRGEPRFIAVGYVDDTQFVRFDSDAASQRMEPRAPWIEQEGPEYWDRNTRNVKAHSQTDRESLRIALRCYNQSEDGSHTIQRMYGCDVGPDGRFLRGYQQDAYDGKDYIALNEDLRSWTAADMCAQITQRKWETAHEAEQWRAYLEGRCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 180 - protein, 276 aa, synthetic construct
  REGION 1..276: note = Synthetic: HLA-A*26:01
GSHSMRYFYTSVSRPGRGEPRFIAVGYVDDTQFVRFDSDAASQRMEPRAPWIEQEGPEYWDRNTRNVKAHSQTDRANLGTLRGCYNQSEDGSHTIQRMYGCDVGPDGRFLRGYQQDAYDGKDYIALNEDLRSWTAADMCAQITQRKWETAHEAEQWRAYLEGRCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 181 - protein, 276 aa, synthetic construct
  REGION 1..276: note = Synthetic: HLA-A*29:02
GSHSMRYFTTSVSRPGRGEPRFIAVGYVDDTQFVRFDSDAASQRMEPRAPWIEQEGPEYWDLQTRNVKAQSQTDRANLGTLRGCYNQSEAGSHTIQMMYGCDVGSDGRFLRGYRQDAYDGKDYIALNEDLRSWTAADMCAQITQRKWEAARVAEQLRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 182 - protein, 276 aa, synthetic construct
  REGION 1..276: note = Synthetic: HLA-A*68:02
GSHSMRYFYTSMSRPGRGEPRFIAVGYVDDTQFVRFDSDAASQRMEPRAPWIEQEGPEYWDRNTRNVKAQSQTDRVDLGTLRGCYNQSEAGSHTIQRMYGCDVGPDGRFLRGYHQYAYDGKDYIALKEDLRSWTAADMCAQTTKHKWEAAHVAEQWRAYLEGTCVEWLRRYLENGKETLQRTDAPKTHMTHHAVSDHEATLRCWALSFYPAEITLTWQRDGEDQTQDTELVETRPAGDGTFQKWVAVVVPSGQEQRYTCHVQHEGLPKPLTLRWEP

SEQ ID NO: 183 - protein, 276 aa, synthetic construct
  REGION 1..276: note = Synthetic: HLA-B*13:02
GSHSMRYFYTAMSRPGRGEPRFITVGYVDDTQFVRFDSDATSPRMAPRAPWIEQEGPEYWDRETQISKTNTQTYRENLRTALRCYNQSEAGSHTWQTMYGCDLGPDGRLLRGHNQLAYDGKDYIALNEDLSSWTAADTCAQITQLKWEAARVAEQLRAYLEGECVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 184 - protein, 276 aa, synthetic construct
  REGION 1..276: note = Synthetic: HLA-B*15:07
GSHSMRYFYTAMSRPGRGEPRFIAVGYVDDTQFVRFDSDAASPRMAPRAPWIEQEGPEYWDRETQISKTNTQTYRESLRNLRGCYNQSEAGSHTLQSMYGCDVGPDGRLLRGHDQSAYDGKDYIALNEDLSSWTAADTCAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 185 - protein, 276 aa, synthetic construct
  REGION 1..276: note = Synthetic: HLA-B*27:05
GSHSMRYFHTSVSRPGRGEPRFITVGYVDDTLFVRFDSDAASPREEPRAPWIEQEGPEYWDRETQICKAKAQTDREDLRTLLRCYNQSEAGSHTLQNMYGCDVGPDGRLLRGYHQDAYDGKDYIALNEDLSSWTAADTCAQITQRKWEAARVAEQLRAYLEGECVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 186 - protein, 276 aa, synthetic construct
  REGION 1..276: note = Synthetic: HLA-B*35:03
GSHSMRYFYTAMSRPGRGEPRFIAVGYVDDTQFVRFDSDAASPRTEPRAPWIEQEGPEYWDRNTQIFKTNTQTYRESLRNLRGCYNQSEAGSHIIQRMYGCDLGPDGRLLRGHDQFAYDGKDYIALNEDLSSWTAADTCAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 187 - protein, 276 aa, synthetic construct
  REGION 1..276: note = Synthetic: HLA-B*37:01
GSHSMRYFHTSVSRPGRGEPRFISVGYVDDTQFVRFDSDAASPRTEPRAPWIEQEGPEYWDRETQISKTNTQTYREDLRTLLRCYNQSEAGSHTIQRMSGCDVGPDGRLLRGYNQFAYDGKDYIALNEDLSSWTAADTCAQITQRKWEAARVAEQDRAYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 188 - protein, 276 aa, synthetic construct
  REGION 1..276: note = Synthetic: HLA-B*38:01
GSHSMRYFYTSVSRPGRGEPRFISVGYVDDTQFVRFDSDAASPREEPRAPWIEQEGPEYWDRNTQICKTNTQTYRENLRIALRCYNQSEAGSHTLQRMYGCDVGPDGRLLRGHNQFAYDGKDYIALNEDLSSWTAADTCAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 189 - protein, 276 aa, synthetic construct
  REGION 1..276: note = Synthetic: HLA-B*41:02
GSHSMRYFHTAMSRPGRGEPRFITVGYVDDTLFVRFDSDATSPRKEPRAPWIEQEGPEYWDRETQISKTNTQTYRESLRNLRGCYNQSEAGSHTLQSMYGCDVGPDGRLLRGHNQYAYDGKDYIALNEDLRSWTAADTCAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 190 - protein, 276 aa, synthetic construct
  REGION 1..276: note = Synthetic: HLA-B*44:05
GSHSMRYFYTAMSRPGRGEPRFITVGYVDDTLFVRFDSDATSPRKEPRAPWIEQEGPEYWDRETQISKTNTQTYRENLRTALRCYNQSEAGSHIIQRMYGCDVGPDGRLLRGYDQYAYDGKDYIALNEDLSSWTAADTCAQITQRKWEAARVAEQDRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEP

SEQ ID NO: 191 - protein, 276 aa, synthetic construct
  REGION 1..276: note = Synthetic: HLA-B*49:01
GSHSMRYFHTAMSRPGRGEPRFITVGYVDDTLFVRFDSDATSPRKEPRAPWIEQEGPEYWDRETQISKTNTQTYRENLRIALRCYNQSEAGSHTWQRMYGCDLGPDGRLLRGYNQLAYDGKDYIALNEDLSSWTAADTCAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 192 - protein, 276 aa, synthetic construct
  REGION 1..276: note = Synthetic: HLA-B*52:01
GSHSMRYFYTAMSRPGRGEPRFIAVGYVDDTQFVRFDSDAASPRTEPRAPWIEQEGPEYWDRETQISKTNTQTYRENLRIALRCYNQSEAGSHTWQTMYGCDVGPDGRLLRGHNQYAYDGKDYIALNEDLSSWTAADTCAQITQRKWEAAREAEQLRAYLEGLCVEWLRRHLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 193 - protein, 276 aa, synthetic construct
  REGION 1..276: note = Synthetic: HLA-B*55:01
GSHSMRYFYTAMSRPGRGEPRFIAVGYVDDTQFVRFDSDAASPREEPRAPWIEQEGPEYWDRNTQIYKAQAQTDRESLRNLRGCYNQSEAGSHTWQTMYGCDLGPDGRLLRGHNQLAYDGKDYIALNEDLSSWTAADTCAQITQRKWEAAREAEQLRAYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 194 - protein, 276 aa, synthetic construct
  REGION 1..276: note = Synthetic: HLA-C*03:03
GSHSMRYFYTAVSRPGRGEPHFIAVGYVDDTQFVRFDSDAASPRGEPRAPWVEQEGPEYWDRETQKYKRQAQTDRVSLRNLRGCYNQSEARSHIIQRMYGCDVGPDGRLLRGYDQYAYDGKDYIALNEDLRSWTAADTCAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 195 - protein, 276 aa, synthetic construct
  REGION 1..276: note = Synthetic: HLA-C*07:04
CSHSMRYFDTAVSRPGRGEPRFISVGYVDDTQFVRFDSDAASPRGEPRAPWVEQEGPEYWDRETQKYKRQAQADRVSLRNLRGCYNQSEDGSHTFQRMYGCDLGPDGRLLRGYDQFAYDGKDYIALNEDLRSWTAADTCAQITQRKLEAARAAEQDRAYLEGTCVEWLRRYLENGKKTLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 196 - protein, 276 aa, synthetic construct
  REGION 1..276: note = Synthetic: HLA-C*08:01
CSHSMRYFYTAVSRPGRGEPRFIAVGYVDDTQFVQFDSDAASPRGEPRAPWVEQEGPEYWDRETQKYKRQAQTDRVSLRNLRGCYNQSEAGSHTLQRMYGCDLGPDGRLLRGYNQFAYDGKDYIALNEDLRSWTAADTCAQITQRKWEAARTAEQLRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGP

SEQ ID NO: 197 - protein, 276 aa, synthetic construct
  REGION 1..276: note = Synthetic: HLA-C*08:02
CSHSMRYFYTAVSRPGRGEPRFIAVGYVDDTQFVQFDSDAASPRGEPRAPWVEQEGPEYWDRETQKYKRQAQTDRVSLRNLRGCYNQSEAGSHTLQRMYGCDLGPDGRLLRGYNQFAYDGKDYIALNEDLRSWTAADKCAQITQRKWEAAREAEQRRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGP

SEQ ID NO: 198 - protein, 276 aa, synthetic construct
  REGION 1..276: note = Synthetic: HLA-C*12:02
CSHSMRYFYTAVSRPGRGEPRFIAVGYVDDTQFVRFDSDAASPRGEPRAPWVEQEGPEYWDRETQKYKRQAQADRVSLRNLRGCYNQSEAGSHTLQRMYGCDLGPDGRLLRGYDQSAYDGKDYIALNEDLRSWTAADTCAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 199 - protein, 276 aa, synthetic construct
  REGION 1..276: note = Synthetic: HLA-C*12:03
CSHSMRYFYTAVSRPGRGEPRFIAVGYVDDTQFVRFDSDAASPRGEPRAPWVEQEGPEYWDRETQKYKRQAQADRVSLRNLRGCYNQSEAGSHTLQWMYGCDLGPDGRLLRGYDQSAYDGKDYIALNEDLRSWTAADTCAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 200 - protein, 276 aa, synthetic construct
  REGION 1..276: note = Synthetic: HLA-C*14:02
CSHSMRYFSTSVSRPGRGEPRFIAVGYVDDTQFVRFDSDAASPRGEPRAPWVEQEGPEYWDRETQKYKRQAQTDRVSLRNLRGCYNQSEAGSHTLQWMFGCDLGPDGRLLRGYDQSAYDGKDYIALNEDLRSWTAADTCAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 201 - protein, 276 aa, synthetic construct
  REGION 1..276: note = Synthetic: HLA-C*15:02
CSHSMRYFYTAVSRPGRGEPHFIAVGYVDDTQFVRFDSDAASPRGEPRAPWVEQEGPEYWDRETQNYKRQAQTDRVNLRKLRGCYNQSEAGSHIIQRMYGCDLGPDGRLLRGHDQLAYDGKDYIALNEDLRSWTAADTCAQITQRKWEAAREAEQLRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 202 - protein, 276 aa, synthetic construct
  REGION 1..276: note = Synthetic: HLA-C*17:01
GSHSMRYFYTAVSRPGRGEPRFIAVGYVDDTQFVRFDSDAASPRGEPRAPWVEQEGPEYWDRETQKYKRQAQADRVNLRKLRGCYNQSEAGSHTIQRMYGCDLGPDGRLLRGYNQFAYDGKDYIALNEDLRSWTAADTCAQISQRKLEAAREAEQLRAYLEGECVEWLRGYLENGKETLQRAERPKTHVTHHPVSDHEATLRCWALGFYPAEITLTWQRDGEDQTQDTELVETRPAGDGTFQKWAAVVVPSGQEQRYTCHVQHEGLQEPCTLRWKP